SEQUENCE LISTING (45 sequences)

SEQ ID NO: 1 - DNA, 17 bp, Artificial Sequence
  COVID-19 (SARS-Cov-2)  N2 forward primer of the oligo primers (N1 forward primer) (20-mer, nt 28863-28882, MN908947.3)
caactccaggcagtagg

SEQ ID NO: 2 - DNA, 21 bp, Artificial Sequence
  N2 reverse primer of the oligo primers (N1 reverse primer) (21-mer, nt 28960-28980, MN908947.3)
ccagacattttgctctcaagc

SEQ ID NO: 3 - DNA, 25 bp, Artificial Sequence
  Orf1ab forward primer of the oligo primers (ORF1ab forward primer) (25-mer, nt 13348-13372,MN908947.3)
gggttttacacttaaaaacacagtc

SEQ ID NO: 4 - DNA, 20 bp, Artificial Sequence
  Orf1ab reverse primer of the oligo primers (ORF1ab reverse primer) (20-mer, nt 13433-13452, MN908947.3)
gcatcagctgactgaagcat

SEQ ID NO: 5 - DNA, 20 bp, Artificial Sequence
  RNA dependent RNA polymerase forward primer of the oligo primers (RdRP forward primer) (20-mer, nt 15441-1 5460, MN908947.3)
catgtgtggcggttcactat

SEQ ID NO: 6 - DNA, 27 bp, Artificial Sequence
  RdRP reverse primer of the oligo primers (RdRP reverse primer) (27-mer, nt 15500-15526, MN908947.3)
tgttaaaaacactattagcataagcag

SEQ ID NO: 7 - DNA, 21 bp, Homo sapiens
  misc_feature: Human internal control-1 forward primer of the oligo primers (beta-actin forward primer, 21-mer, nt 34 2-362, from NM_001101.5)
gcaccacaccttctacaatga

SEQ ID NO: 8 - DNA, 20 bp, Homo sapiens
  misc_feature: Human internal control-1 reverse primer of the oligo primers (beta-actin reverse primer, 20-mer, nt 42 4-443 from NM_001101.5)
gtcatcttctcgcggttggc

SEQ ID NO: 9 - DNA, 77 bp, Artificial Sequence
  Potential site of oligonucleotide probe of N2 (77-mer, nt 28885-28951, NM_001101.5)
ggaacttctcctgctagaatggctggcaatggcggtgatgctgctcttgctttgctgctgcttgacagattgaacca

SEQ ID NO: 10 - DNA, 20 bp, Artificial Sequence
  oligonucleotide probe of N2 (20-mer, nt 28929-28953, MN908947.3)
ttgctgctgcttgacagatt

SEQ ID NO: 11 - DNA, 20 bp, Artificial Sequence
  Oligonucleotide probe of N2 with designated Reporter and Quencher (20-mer, nt 28937-28956, MN908947.3) and 5′-/TexRed BHQ_2/-3′
ttgctgctgcttgacagatt

SEQ ID NO: 12 - DNA, 60 bp, Artificial Sequence
  Potential site of oligonucleotide probe of ORF1ab (60 mer, 13373-13432, MN908947.3)
tgtaccgtctgcggtatgtggaaaggttatggctgtagttgtgatcaactccgcgaaccc

SEQ ID NO: 13 - DNA, 28 bp, Artificial Sequence
  oligonucleotide probe of ORF1ab (28-mer, nt13377-13406.,MN908947.3)
ccgtctgcggtatgtggaaaggttatgg

SEQ ID NO: 14 - DNA, 28 bp, Artificial Sequence
  5′-Cyanine5 and Oligonucleotide probe of Orf1ab with designated Reporter and Quencher (28-mer, nt13377-13 406.,MN908947.3) or 5′-FAM and Oligonucleotide probe of Orf1ab with designated Reporter and Quencher for Triplex-1 (28-mer,
ccgtctgcggtatgtggaaaggttatgg

SEQ ID NO: 15 - DNA, 39 bp, Artificial Sequence
  Potential site of oligonucleotide probe of RdRp (39-mer, nt 28883-28958,MN908947.3)
atgttaaaaccaggtggaacctcaggagatgccacaaac

SEQ ID NO: 16 - DNA, 25 bp, Artificial Sequence
  oligonucleotide probe of RdRP (25-mer, nt15471-15495, MN908947.3)
caggtggaacctcatcaggagatgc

SEQ ID NO: 17 - DNA, 28 bp, Artificial Sequence
  Oligonucleotide probe of RdRP with designated Reporter & Qencher (25-mer, nt15471-15495,MN908947.3)
ccgtctgcggtatgtggaaaggttatgg

SEQ ID NO: 18 - DNA, 23 bp, Homo sapiens
  misc_feature: Human internal control-1 oligonucleotide probe (21-mer, nt 384-404, from NM_001101.5)
caccccgtgctgctgaccgaggc

SEQ ID NO: 19 - DNA, 23 bp, Artificial Sequence
  Oligonucleotide probe of beta actin with designated Reporter and Quencher (21-mer, nt 384-404, from NM_001 101.5); 5′-[HEX]/ and /[BHQ]-3′
caccccgtgctgctgaccgaggc

SEQ ID NO: 20 - DNA, 22 bp, Artificial Sequence
  COVID-19 (SARS-Cov-2) N gene (N1 region) forward primer of the oligo primers(N1 forward primer)
ggggaacttctcctgctagaat

SEQ ID NO: 21 - DNA, 20 bp, Artificial Sequence
  COVID-19 (SARS-Cov-2)  N gene (N1 region) reverse primer of the  oligo primers (N1 reverse primer)
ttgctctcaagctggttcaa

SEQ ID NO: 22 - DNA, 49 bp, Artificial Sequence
  Potential site of oligonucleotide probe of N-1 (48 mer, 28903-28951, MN908947.3)
ggctggcaatggcggtgatgctgctcttgctttgctgctgcttgacaga

SEQ ID NO: 23 - DNA, 20 bp, Artificial Sequence
  N gene (N-1)  oligonucleotide probe (N1 probe, 20 mer, 28934-28953, MN908947.3)
ttgctgctgcttgacagatt

SEQ ID NO: 24 - DNA, 20 bp, Artificial Sequence
  COVID-19 (SARS-Cov-2)  N gene (N-1) oligonucleotide probe(N1 probe) with designated Reporter and Quencher (N1 probe, 20 mer, 28934-28953, MN908947.3) 5′-TexasRed and -BHQ2-3′
ttgctgctgcttgacagatt

SEQ ID NO: 25 - DNA, 20 bp, Artificial Sequence
  (internal control gene-2) Pbgd forward primer of the oligo primers( (Pbgd forward primer) (20-mer, nt 21-40, from  NM_000190.4
gagaccaggagtcagactgt

SEQ ID NO: 26 - DNA, 20 bp, Artificial Sequence
  (internal control gene-2) Pbgd reverse primer of the oligo primers (Pbgd reverse primer) (20-mer, nt 128-147 from NM_000190.4)
gcttggaaagtaggctgtgt

SEQ ID NO: 27 - DNA, 23 bp, Homo sapiens
  misc_feature: (internal control gene-2) Human internal control-2 oligonucleotide probe / oligonucleotide probe of Pbgd (20-mer, nt 58-75 from NM_000190.4)
caccccgtgctgctgaccgaggc

SEQ ID NO: 28 - DNA, 18 bp, Artificial Sequence
  internal control gene-2) oligonucleotide probe of Pbgd with designated Reporter and Quencher (18-mer, nt 58-75 from NM_000190.4) 5′-[HEX]/ and /[BHQ1]- 3
cacgtgtccccggtactc

SEQ ID NO: 29 - DNA, 102 bp, Artificial Sequence
  Plasmid RNA-derived internal control -1 sequence for beta actin marker (102 mer)
ggcaccacaccttctacaatgagctgcgtgtggctcccgaggagcaccccgtgctgctgaccgaggcccccctgaaccccaaggccaaccgcgagaagatga

SEQ ID NO: 30 - DNA, 127 bp, Artificial Sequence
  Plasmid RNA-derived internal control -2 sequence for Pbgd marker (127 mer)
gagaccaggagtcagactgtaggacgacctcgggtcccacgtgtccccggtactcgccggccggagcccccggcttcccggggccgggggaccttagcggcacccacacacagcctactttccaagc

SEQ ID NO: 31 - DNA, 119 bp, Artificial Sequence
  Plasmid RNA-derived Positive control sequence for Orf1ab (119 mer)
ccctgtgggttttacacttaaaaacacagtctgtaccgtctgcggtatgtggaaaggttatggctgtagttgtgatcaactccgcgaacccatgcttcagtcagctgatgcacaatcgt

SEQ ID NO: 32 - DNA, 86 bp, Artificial Sequence
  RdRP target sequence
catgtgtggcggttcactatatgttaaaccaggtggaacctcatcaggagatgccacaactgcttatgctaatagtgtttttaaca

SEQ ID NO: 33 - DNA, 105 bp, Artificial Sequence
  Orf1ab target sequence
gggttttacacttaaaaacacagtctgtaccgtctgcggtatgtggaaaggttatggctgtagttgtgatcaactccgcgaacccatgcttcagtcagctgatgc

SEQ ID NO: 34 - DNA, 91 bp, Artificial Sequence
  N-1 target sequence
ggggaacttctcctgctagaatggctggcaatggcggtgatgctgctcttgctttgctgctgcttgacagattgaaccagcttgagagcaa

SEQ ID NO: 35 - DNA, 118 bp, Artificial Sequence
  N-2 target sequence
caactccaggcagcagtaggggaacttctcctgctagaatggctggcaatggcggtgatgctgctcttgctttgctgctgcttgacagattgaaccagcttgagagcaaaatgtctgg

SEQ ID NO: 36 - DNA, 102 bp, Artificial Sequence
  Beta actin target sequence
gcaccacaccttctacaatgagctgcgtgtggctcccgaggagcaccccgtgctgctgaccgaggcccccctgaaccccaaggccaaccgcgagaagatggc

SEQ ID NO: 37 - DNA, 127 bp, Artificial Sequence; identical to SEQ ID NO: 30
  Pbgd target sequence

SEQ ID NO: 38 - DNA, 20 bp, Artificial Sequence
  N3 probe (20 mer), 28905-28924 bp
ctggcaatggcggtgatgct

SEQ ID NO: 39 - DNA, 23 bp, Artificial Sequence
  N4 probe (23mer) 28905-28927 bp
ctggcaatggcggtgatgctgct

SEQ ID NO: 40 - DNA, 22 bp, Artificial Sequence
  40 - N5 probe (22 mer) 28909 -28930 bp
caatggcggtgatgctgctctt

SEQ ID NO: 41 - DNA, 21 bp, Artificial Sequence
  - N6 probe3 (21 mer) 28919-28939 bp
gatgctgctcttgctttgctg

SEQ ID NO: 42 - DNA, 20 bp, Artificial Sequence
  Beta-actin/Internal control-2, forward primer, (20 mer] 985-1004 bp
ggcggcaccaccatgtaccc

SEQ ID NO: 43 - DNA, 20 bp, Artificial Sequence
  Beta-actin/Internal control-2 reverse primer (20 mer] 1169-1188 bp
ggaggggccggactcgtcat

SEQ ID NO: 44 - DNA, 21 bp, Artificial Sequence
  eta-actin/Internal control-2 primers and probe [20 mer] 1107-1127 bp
cggcggctccatcctggcctc

SEQ ID NO: 45 - DNA, 99 bp, Artificial Sequence
  Plasmid RNA-derived Positive control sequence for N marker (99 mer)
ggggaacttctcctgctagaatggctggcaatggcggtgatgctgctcttgctttgctgctgcttgacagattgaaccagcttgagagcaaaatgtctg

(19) United States
(12) Patent Application Publication
(10) Pub. No.: US 2023/0009218 A1
(43) Pub. Date: Jan. 12, 2023

(54) Primer, Probe And Controls For Detection And Discrimination Of Covid-19 And Other Coronaviruses

(71) Applicants: Jung Joo Moon, New York, NY (US); Sung Woo Moon, Seoul (KR)

(72) Inventors: Jung Joo Moon, New York, NY (US); Sung Woo Moon, Seoul (KR)

(21) Appl. No.: 17/316,679

(22) Filed: May 10, 2021

Related U.S. Application Data
(60) Provisional application No. 63/022,403, filed on May 8, 2020.

(51) Int. Cl. C12Q 1/70

(52) U.S. Cl. CPC C12Q 1/701

(57) ABSTRACT

The present invention relates to a diagnostic assay for the virus causing severe acute respiratory syndrome Sars-CoV 2 (COVID-19, COVID-19; COVID-19-CoV-2) in humans (“COVID-19 virus”). In particular, the invention relates to a real-time quantitative PCR assay for the detection of COVID-19 virus using reverse transcription and polymerase chain reaction. Specifically, the qualitative assay is a TaqMan® assay using the primers and probes constructed based on the genome of the COVID-19 virus. The invention further relates to a diagnostic kit that comprises nucleic acid molecules for the detection of the COVID-19 virus.

### 1. CROSS-REFERENCE INFORMATION

This application claims priority from U.S. Provisional application Ser. No. 63/022,403, filed on May 8, 2020, which is hereby incorporated herein by reference in its entirety.

The Sequence Listing, which is a part of the present disclosure, includes a computer readable form and a written sequence listing comprising nucleotide and/or amino acid sequences of the present invention. The sequence listing information recorded in computer readable form is identical to the written sequence listing. The ASCII text file, entitled “COVID-19_diagnosis_seq_2021-05-08_ST25.txt” was created on Jun. 13, 2021 having a size of 13,291 bytes, using PatentIn version 3.5 and is incorporated herein by reference in its entirety. The subject matter of the Sequence Listing is incorporated herein by reference in its entirety.

### 2. FIELD OF THE INVENTION

The present invention relates to a diagnostic assay for the virus causing severe acute respiratory syndrome Sars-CoV 2 (COVID-19, COVID-19; COVID-19-CoV-2) in humans (“Sars-CoV disease 2019”).

### 3. BACKGROUND

Recently, there has been an outbreak of atypical pneumonia in Wuhan province in mainland China, in December, 2019. Coronavirus disease 2019 (COVID-19, 2019-nCoV) is an infectious disease caused by severe acute respiratory syndrome coronavirus 2 (SARS-CoV-2). This new virus and disease are unknown before the outbreak began in Wuhan, China, in December 2019, and quickly spread across the globe resulting in the 2019-20 coronavirus pandemic, as defined by the WHO (World Health Organization). SARS-CoV-2 is found to be a positive-sense, single-stranded RNA virus belonging to the genus Betacoronavirus.

In humans, COVID-19 typically spreads from one person to another via respiratory droplets produced during coughing and sneezing as it contacts respiratory or ocular/nasal/oral mucous membranes or direct contact. Common symptoms include fever, cough, and shortness of breath, and time from exposure to onset of symptoms is generally between two and 14 days. Muscle pain, sputum production and sore throat are less common. While the majority of cases result in mild symptoms, and are sometimes asymptomatic, some progress to severe pneumonia and multi-organ failure. The rate of deaths per number of diagnosed cases (case fatality rate (CFR)) is on average around 3.4%, ranging from 0.2% in those less than 20 to approximately 15% in those over 80 years old.

The WHO (World Health Organization) declared the infectivity of COVID-19 as lower than Severe Acute Respiratory Syndrome (SARS-CoV, coronavirus) but higher than the Middle East Respiratory Syndrome (MERS-CoV, MERS coronavirus) on Jan. 24, 2020. WHO declared the basic reproduction number (RO) of the COVID-19 virus as ranging between 1.4 and 2.92. RO is an indication of the transmissibility of a virus, representing the average number of new infections generated by an infectious person in a totally naïve population. For R0>1, the number infected is likely to increase, as it means that 1 infectious person will transmit disease to more than 1 totally naïve person. For SARS-CoV, R0 is 4, and for MERS-CoV, R0 ranged between 0.4 to 0.9. Thus, on Jan. 30, 2020, the Director-General of the WHO declared that the outbreak of COVID-19 constitutes a Public Health Emergency of International Concern (PHEIC). Soon afterwards, the WHO characterized it as pandemic on Mar. 11, 2020 and current WHO risk assessment is “very high” on a global level. There are a total of 865,585 cases and 48,816 deaths associated with COVID-19 in United States alone (Apr. 24, 2020). Good Gene, InC (South Korea) has developed a molecular assay (real-time RT-PCR, real time Reverse transcriptase Polymerase Chain Reaction) for the rapid and reliable diagnosis of COVID-19, which enables the testing of new ORFlab, RdRP, and N regions of COVID-19 and human internal control genes within a single tube. However, the molecular assays of COVID-19 often have high amounts of false positive and false negative results.

Therefore, there is a high need for an accurate testing method to diagnose the COVID-19, which minimize false positive and false negative results. The systems and methods are directed to the development of a combination of primers and probes that has 100% inclusivity with all reported COVID-19 virus strains.

Additionally, the systems and methods herein address is no homology with the other types of huma3infecting coronaviruses, less than 80% homology with human genome or respiratory pathogens through in silico testing, and more than 7 nucleotides difference with SARS-Covid. Thus, we believe this has no cross reactivity and 100% inclusivity (refer to: attached BLAST 00).

### 4. SUMMARY OF THE INVENTION

The invention relates to the use of the sequence information of isolated COVID-19 virus for diagnostic methods, the isolated sequences of COVID-19 virus is deposited in Genbank, NCBI with Accession No: MN908947.3 (SEQ ID NO: 1-12), which is incorporated herein by reference.

In a specific embodiment, the invention provides a method for diagnostic assay for the COVID-19 virus, natural or artificial variants, analogs, or derivatives thereof. In particular, the diagnostic assay is a qualitative assay for detecting nucleic acid molecules of COVID-19 virus using reverse tran-scription and polymerase chain reaction (RT-PCR) or a semiquantitative testing using a titration curve, wherein the qualitative assay is a TaqMan® assay.

In a specific embodiment, the invention also relates to a method for identifying a subject infected with the COVID-19 virus, natural or artificial variants, analogs, or derivatives thereof. In particular, the method comprises obtaining total RNA from a biological sample, wherein the biological sample is obtained from the subject; reverse transcribing the total RNA to obtain cDNA; and subjecting the cDNA to PCR assay using a set of primers and probes.

In a specific embodiment, the invention includes primers and dual-labeled hydrolysis (Taqman®) probes to be used in the in vitro qualitative detection of COVID-19 virus from RNA isolated from clinical respiratory specimens including nasopharyngeal, oropharyngeal, and nasal swabs, wherein the clinical respiratory specimens comprise upper and lower respiratory specimens. More particularly, the RNA isolated from upper and lower respiratory specimens is purified, reverse transcribed to cDNA, subsequently amplified in a single tube in real time RT-PCR machines and associated software.

In a specific embodiment, the invention relates to a sequence for diagnostic methods used for detecting the COVID-19 virus, in a biological sample via detecting agents, wherein the detecting agents is a COVID-19 virus having a genomic nucleic acid or nucleotides encoded by the nucleic acid sequence of SEQ ID NO:1-37.

In a specific embodiment, the invention relates to nucleic acid molecules that are suitable for hybridization to COVID-19 nucleic acids comprising PCR primers, Reverse Transcriptase primers, probes for Southern analysis or other nucleic acid hybridization analysis for the detection of COVID-19 nucleic acids. Said COVID-19 nucleic acids comprise the nucleic acid sequence of SEQ ID NO: 1-18 or a complement, analog, derivative, or fragment thereof, or a portion thereof; and primers comprising the nucleic acid sequence of one or more of SEQ ID 1, 2, 3, 4, 5, 6, 7, and 8.

In a specific embodiment, the invention relates to the nucleic acid molecules comprising the nucleic acid sequence of SEQ ID NO:1,2,3,4,7,8,13,14, or a portion thereof for detecting the COVID-19 virus in a RT-PCR assay using nucleic acid molecules comprising the nucleic acid sequences of SEQ ID 1,2,3,4,7,8,13,14 as primers, wherein the primers are Qplex.

In a specific embodiment, the invention relates to nucleic acid molecules comprising the nucleic acid sequence of SEQ ID NO:1,2, 7,8,13,14, or a portion thereof for detecting the COVID-19 virus in a RT-PCR assay using nucleic acid molecules comprising the nucleic acid sequences of SEQ ID NO: 1 as primers, wherein the primers are Triplex-2.

In a specific embodiment, the invention relates to nucleic acid molecule comprising the nucleic acid sequence of SEQ ID NO:1,2,3,4,15,16, or a portion thereof, and may be used for the detection of the COVID-19 virus in a RT-PCR assay using nucleic acid molecules comprising the nucleic acid sequences of SEQ ID NO: 1 as primers, wherein the primers are Triplex-1.

In a specific embodiment, the invention relates to a method for detecting activity levels of COVID-19 virus and expression of the COVID-19 virus, natural or artificial variants, analogs, or derivatives thereof, in a sputum, nasopharyngeal aspirates, and so forth, wherein the activity levels are increased activity or decreased activity of the COVID-19 virus or the expression of the COVID-19 virus in a sample relative to a control sample are: determined by contacting the upper and lower respiratory specimens (such as nasopharyngeal or oropharyngeal swabs, sputum, lower respiratory tract aspirates, bronchoalveolar lavage, and nasopharyngeal wash/aspirate or nasal aspirate) with an agent which directly or indirectly detects the activity levels of the COVID-19 virus or the expression of the COVID-19 virus; and using detecting agents, wherein the detecting agents comprise nucleic acid molecules.

In specific embodiment, the invention relates to the detecting nucleic acid molecules are immobilized on a DNA microarray chip.

In a specific embodiment, the invention relates a diagnostic kit comprising a nucleic acid molecule for detecting a COVID-19 virus, natural or artificial variants, analogs, or derivatives thereof, wherein the nucleic acid molecule has nucleic acid sequence of SEQ ID 1, 2, 3, 4, 7, 8, 13, 14, 9, 10, 11, 18; the nucleic acid molecule has the nucleic acid sequence of SEQ ID NO: 1, 2, 7, 8, 13, 14, 9, 12, 18; the nucleic acid molecule has the nucleic acid sequence of SEQ ID NO: 1, 2, 3, 4, 15, 16, 9, 11, 17; the nucleic acid molecule has the nucleic acid sequence of SEQ ID NO:1, 2, 9; the nucleic acid molecule has the nucleic acid sequence of any combinations of three nucleic acid sequences: (i) SEQ ID NO. 1, 2, 10 or 11; (ii) SEQ ID NO: 3, 4, 13 or 14); (iii) Seq ID NO: 5, 6, 18 or 19; (iv) COVID markers, wherein the COVID marker are SEQ ID NO: 20,21, 23 or 24); (v) Seq ID NO: 7,8,18 or 19; and (vi) a human internal control, wherein the human internal control is Seq ID NO: 25, 26, 27 or 28.

In a specific embodiment, the invention relates to the diagnostic kit comprises primers and a specific probe, wherein the primers comprise QPlex PCR, Triplex-1, Triplex-2, Duplex-1/2/3, and single PCR and the specific probe comprises a RdRp specific probe, a N-2 specific probe, an Orflab specific probe, an internal control beta actin specific probe, and an internal control Pbgd specific probe.

In a specific embodiment, the invention relates to the RdRP specific probe such that a signal from a fluorescent dye on a 5′ end is quenched by BHQ-1 on a 3′ end, wherein the fluorescent dye is FAM.

In a specific embodiment, the invention relates to the N-2 specific probe such that a signal from a fluorescent dye on a 5′ end is quenched by BHQ-2 on a 3′ end, wherein the fluorescent dye is Texas Red.

In a specific embodiment, the invention relates to the Orflab specific probe such that a signal from the fluorescent dye (Cy-5) on the 5′ end is quenched by BHQ-1 on a 3′ end.

In a specific embodiment, the invention relates to the internal control beta actin specific probe, the signal from the fluorescent dye on a 5′ end is quenched by BHQ-1 on a 3′ end, wherein the fluorescent dye is Hex.

In a specific embodiment, the invention relates to triplex-1 for the Orflab specific probe such that a signal from FAM on a 5′ end is quenched by BHQ-1 on a 3′ end; triplex-1 for the N specific probe such that a signal from Texas Red on a 5′ end is quenched by BHQ-2 on a 3′ end; triplex1 for the internal control Pbgd specific probe such that signal from the fluorescent dye (Hex) on a 5′ end is quenched by BHQ-1 on a 3′ end.

In a specific embodiment, the invention relates to the diagnostic kit comprise a multiplex real time PCR machine compatible with the diagnostic kit, wherein the multiplex real time PCR machine comprises: Rotor-Gene Q 5 plex HRM Real Time PCR cycler (Qiagen), CFX96 Real Time PCR Detection System (Bio-Rad), Applied Biosystems 7500 Real Time PCR System (Thermo Fisher Scientific), LineGene 9600 Plus real-time PCR detection system (FQD-96A, Bioer).

In a specific embodiment, the invention relates to the diagnostic kit comprising a comparison genome, wherein the comparison genome is GenBank MN908947.3 SARS-Cov-2 genome for determining locations of each gene markers, wherein the locations are: (i) Triplex-2, QPlex, Duplex, N-2 at 28863-28980 base pairs (bp), (ii) N-1 at 28881-28971 bp; (iii) ORFlab at 13348-13452 bp; and (iv) RdRp at 15441-15526 bp.

### 3.1. Drawings

The patent or application file contains at least one drawing executed in color. Copies of this patent or patent application publication with color drawing(s) will be provided by the Office upon request and payment of the necessary fee.

FIG. 1 is a depiction of the Results of QPlex reverse transcription real time PCR assay of N, ORFlab and RdRp of SARS-Cov-2 and beta-actin in 10-fold serial dilution of SARS-Cov-2 RNA (GENOMIC RNA from MT007544.1 and MN908947.3) and in vitro transcript of beta actin. The green assay corresponds to RdRp, the orange assay corresponds to N, red assay corresponds to ORF, and yellow assay correspond to beta-actin.

FIGS. 2 and 3 are depictions of Actin Beta and hydroxymethylbilane synthase data corresponding to Table 11 for Seq ID NO: 29 and Table 12 for Seq ID NO: 30, respectively.

FIG. 4 is a depiction of a flowchart for preparing and packaging the diagnostic kit.

FIGS. 5A and 5B are depictions and Duplex-2, respectively. The green assay corresponds to RdRp, the orange assay corresponds to N, red assay corresponds to ORF, and yellow assay correspond to beta-actin.

FIG. 6 is a depiction of fluorescence of Duplex-2.

FIGS. 7, 8, and 9 are depictions of quantitative analysis of Duplex 3, Triplex-1, and Triplex-2, respectively. The green assay corresponds to RdRp, the orange assay corresponds to N, red assay corresponds to ORF, and yellow assay correspond to beta-actin.

FIG. 10A is a depiction of an example of “Covid-19 Negative” Analysis of Result of Reverse Transcription Real Time PCR by Using COVID-19 Quadplex RT Real Time PCR Kit.

FIG. 10B is a depiction of an example of “Covid-19 Positive” Analysis of Result of Reverse Transcription Real Time PCR by Using COVID-19 Quadplex RT Real Time PCR Kit (GGCOV-Q).

FIG. 10C is a depiction of an example of “Covid-19 Presumptive Positive” Analysis of Result of Reverse Transcription Real Time PCR by Using COVID-19 Quadplex RT Real Time PCR Kit.

FIG. 10D is a depiction of an example of “Inconclusive Result” Analysis of Result of Reverse Transcription Real Time PCR by Using COVID-19 Quadplex RT Real Time PCR Kit.

FIG. 10E is a depiction of an example of “Invalid Test, Retest Required” Analysis of Result of Reverse Transcription Real Time PCR by Using COVID-19 Quadplex RT Real Time PCR Kit.

FIG. 11A is a depiction of LoD study resulting from 10-fold serial dilution of SARS-Cov-2 RNA (GENOMIC RNA from MT007544.1 and MN908947.3).

FIG. 11B is a depiction of a plot of results from a linearity experiment to determine reportable range where: x=Log 10 concentration of genomic RNA (copies/uL) and y=Ct value obtained.

FIG. 11C is a depiction of a LoD study results from 3-fold dilution of SARS-Cov-2 RNA (GENOMIC RNA from MT007544.1 and MN908947.3) in concentration of 1, 3, 10, 30 and 100 cp/ul.

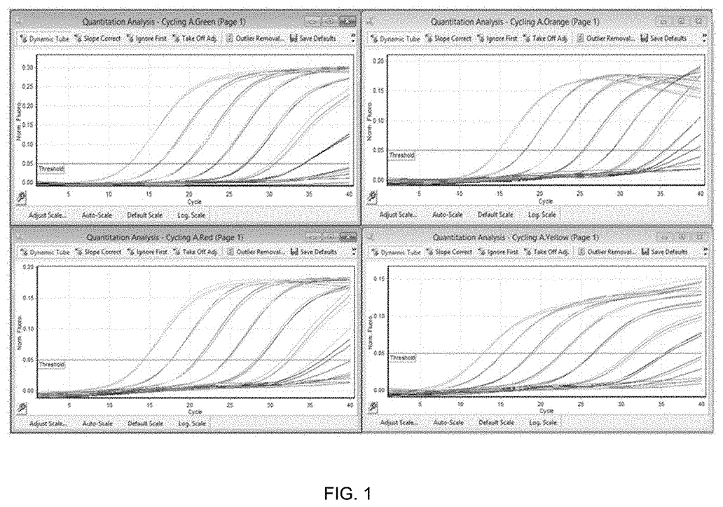

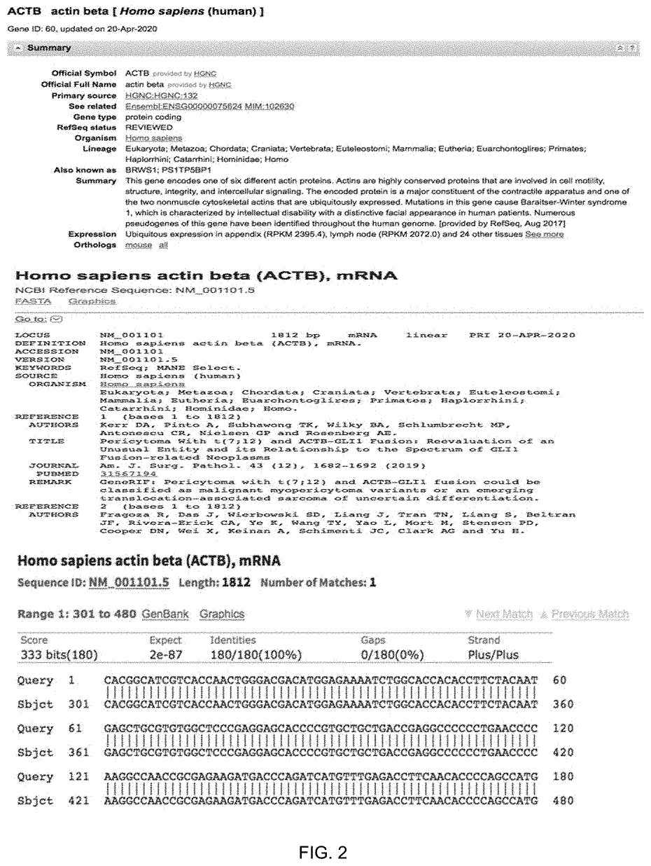

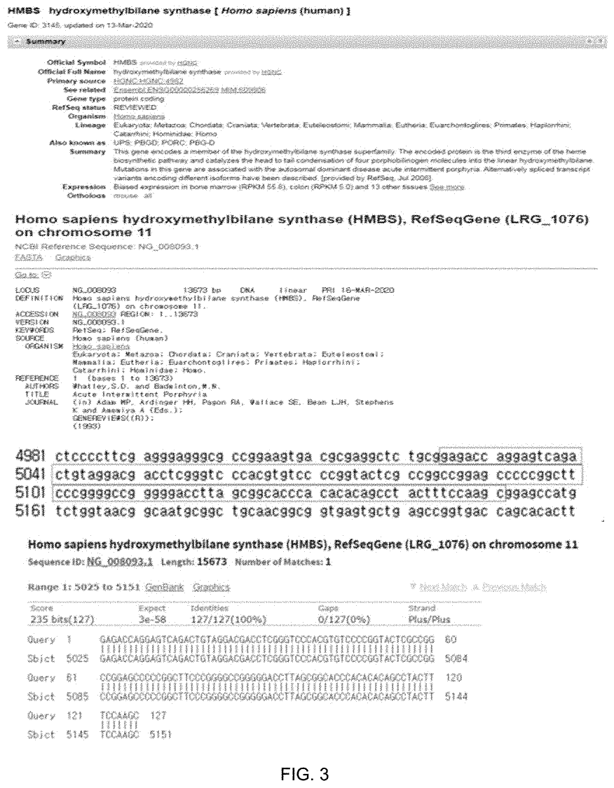

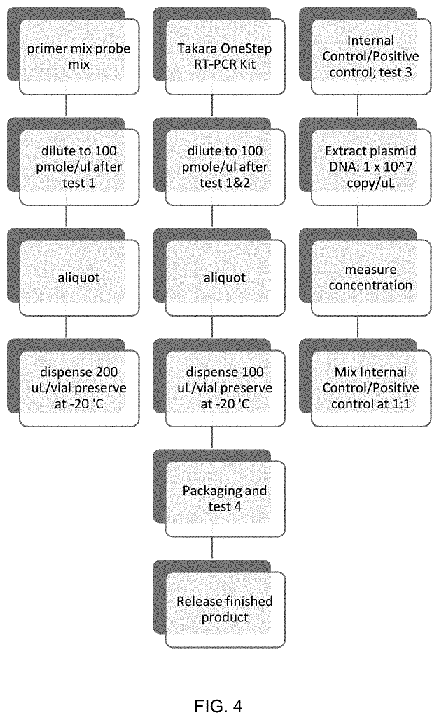

### 5. DETAILED DESCRIPTION OF THE INVENTION

### Definitions

As used herein, the term “variant” refers either to a naturally occurring genetic mutant of the COVID-19 virus or a recombinantly prepared variation of the COVID-19 virus, each of which contain one or more mutations in its genome compared to the COVID-19 virus of MN908947.3. The term “variant” may also refer to either a naturally occurring variation of a given peptide or a recombinantly prepared variation of a given peptide or protein in which one or more amino acid residues have been modified by amino acid substitution, addition, or deletion. As used herein, the term “analogue” in the context of a non-proteinaceous analog refers to a second organic or inorganic molecule which possess a similar or identical function as a first organic or inorganic molecule and is structurally similar to the first organic or inorganic molecule and is structurally similar to the first organic or inorganic molecule. As used herein, the term “derivative” in the context of a non-proteinaceous derivative refers to a second organic or inorganic molecule that is formed based upon the structure of a first organic or inorganic molecule. A derivative of an organic molecule includes, but is not limited to, a molecule modified, e.g., by the addition or deletion of a hydroxyl, methyl, ethyl, carboxyl or amine group. An organic molecule may also be esterified, alkylated and/or phosphorylated.

As used herein, the term “mutant” refers to the presence of mutations in the nucleotide sequence of an organism as compared to a wild-type organism. An “isolated” nucleic acid molecule is one which is 30 separated from other nucleic acid molecules which are present in the natural source of the nucleic acid molecule.

Moreover, an “isolated” nucleic acid molecule, such as a cDNA molecule, can be substantially free of other cellular material, or culture medium when produced by recombinant techniques, or substantially free of chemical precursors or other chemicals when chemically synthesized. In a preferred embodiment of the invention, nucleic acid molecules encoding polypeptides/proteins of the invention are isolated or purified. The term “isolated” nucleic acid molecule does not include a nucleic acid that is a member of a library that has not been purified away from other library clones containing other nucleic acid molecules.

As used herein, the term “isolated” virus is one which is separated from other organisms which are present in the natural source of the virus, e.g., biological material such as cells, blood, serum, plasma, saliva, urine, stool, sputum, etc. and can be used to infect a subject.

As used herein, the term “having a biological activity of the polypeptides of the invention” refers to the characteristics of the polypeptides or proteins having a common biological activity similar or identical structural domain and/or having sufficient amino acid identity to the polypeptide encoded by the nucleotide sequence of SEQ ID NO:1-24, or a variant, analog, derivative, or fragment thereof.

The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:22642268, modified as in Karlin and Altschul, 1993, Proc. Natl. Acad. Sci. U.S.A. 90:58735877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, word length=12 to obtain nucleotide sequences homologous to nucleic acid molecules of the present invention. BLAST protein searches can be performed with the XBLAST program parameters set, e.g., to score 50, word length=3 to obtain amino acid sequences homologous to a protein molecule of the present invention. To obtain gapped alignments for comparison purposes, Gapped BLAST can be utilized as described in Altschul et al., 1997, Nucleic Acids Res. 25:33893402. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., the NCBI website). Another preferred, non-limiting example of a mathematical algorithm utilized for the comparison of sequences is the algorithm of Myers and Miller, 1988, CABIOS 4:11 17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

As used herein, the term “derivative” in the context of proteinaceous agent (e.g., proteins, polypeptides, peptides, and antibodies) refers to a proteinaceous agent that comprises an amino acid sequence which has been altered by the introduction of amino acid residue substitutions, deletions, and/or additions. The term “derivative” as used herein also refers to a proteinaceous agent which has been modified, i.e., by the covalent attachment of any type of molecule to the proteinaceous agent. For example, but not by way of limitation, an antibody may be modified, e.g., by glycosylation, acetylation, pegylation, phosphorylation, amidation, derivatization by known protecting-blocking groups, proteolytic cleavage, linkage to a cellular ligand or other protein, etc. A derivative of a proteinaceous agent may be produced by chemical modifications using techniques known to those of skill in the art, including, but not limited to specific chemical cleavage, acetylation, formylation, metabolic synthesis of tunicamycin, etc. Further, a derivative of a proteinaceous agent may contain one or more non-classical amino acids. A derivative of a proteinaceous agent possesses a similar or identical function as the proteinaceous agent from which it is derived.

As used herein, the terms “subject” and “patient” are used interchangeably. As used herein, the terms “subject” and “subjects” refer to an animal, preferably a mammal including a non-primate (e.g., cows, pigs, horses, goats, sheep, cats, dogs, avian species, and rodents) and a non-primate (e.g., monkeys such as a cynomolgus monkey and humans), and more preferably a human.

Coronaviruses (e.g., COVID-19) are a large family of viruses that comprise: 4 genus (alpha, beta, gamma, delta) which may cause illness in animals or humans. Seven strains of human coronaviruses are known.

The present invention relates to systems and methods herein directed to the use of the sequence information of some sequences the isolated COVID-19 virus for diagnostic methods, which lead to GG COVID-19 Quadplex Real Time RT-PCR. In particular, the GG COVID-19 Quadplex Real Time RT-PCR of the systems and methods here provides a method for detecting the presence or absence of nucleic acid molecules of the COVID-19 virus, natural or artificial variants, analogs, or derivatives thereof, in a biological sample. The method involves obtaining a biological sample from various sources and contacting the sample with a compound or an agent capable of detecting a nucleic acid (e.g., mRNA, genomic DNA) of the COVID-19 virus, natural or artificial variants, analogs, or derivatives thereof, such that the presence of the COVID-19 virus, natural or artificial variants, analogs, or derivatives thereof, is detected in the sample. An agent for detecting COVID-19 mRNA or genomic RNA is a labeled nucleic acid probe capable of hybridizing to mRNA or genomic RNA. In particular, the systems and methods herein relate to: a qualitative assay for the detection of the COVID-19 virus, natural or artificial variants, analogs, or derivatives thereof, using reverse transcription and polymerase chain reaction (RT-PCR), wherein the qualitative assay is a TaqMan® assay; and a diagnostic kit that comprises of nucleic acid molecules for the detection of the COVID-19 virus; and a set/combination of reagents that comprises of reagents, controls, and nucleic acid molecules for the detection of the COVID-19 virus.

In the systems and methods herein, the nucleic acid probe is a nucleic acid molecule comprising or consisting of the nucleic acid sequence of SEQ ID 1-18, or a portion thereof, which sufficiently specifically hybridizes under stringent conditions to an COVID-19 mRNA or genomic RNA.

In the systems and methods herein, the presence of the COVID-19 virus, natural or artificial variants, analogs, or derivatives thereof, is detected in the sample by a Real Time reverse transcription polymerase chain reaction (Real Time RT-PCR) using the primers that are constructed based on a partial nucleotide sequence of the COVID-19 virus.

In the systems and methods herein, the primers and probes are designed by comparison between the genomes of alpha-coronaviruses (HCoV-229E, -NL63), beta-coronaviruses (OC43, -HKU1), SARS-CoV, MERS-CoV, and COVID-19 (Wuhan-Hu-1, complete genome, GenBank: MN908947.3).

In the systems and methods herein, the isolated COVID-19 virus is used for diagnostic methods by: (i) detecting mRNA or genomic RNA of the COVID-19 virus of the in upper and lower respiratory specimens (e.g., nasopharyngeal or oropharyngeal swabs, sputum, lower respiratory tract aspirates, bronchoalveolar lavage, and nasopharyngeal wash/aspirate or nasal aspirate) and so forth; (ii) determining if there is an increased or decreased level of mRNA or genomic RNA of the COVID-19 virus in a sample relative to a control sample by contacting the upper and lower respiratory specimens (such as nasopharyngeal or oropharyngeal swabs, sputum, lower respiratory tract aspirates, bronchoalveolar lavage, and nasopharyngeal wash/aspirate or nasal aspirate) with an agent which can detect directly or indirectly the mRNA or genomic RNA of the COVID-19 virus.

In the systems and methods herein, the detecting agents are the nucleic acid molecules of the present invention, wherein the detecting nucleic acid molecules are immobilized on a DNA microarray chip.

In the systems and methods herein, the nucleic acid sequence (SEQ ID NO: 1-37) for diagnostic methods are used for detecting the COVID-19 virus in a biological sample. More particularly, the detecting agents are a COVID-19 virus, for example, of deposit no. MN908947.3, or having a genomic nucleic acid sequence of SEQ ID 1-37, or nucleotides encoded by the nucleic acid sequence of SEQ ID NO: 1-37.

In the systems and methods herein, a plurality of reagents, which in combination with a nucleotide of the invention encoded by the nucleotide sequence of SEQ ID NO:1-37, or encoded by a nucleic acid comprising a nucleotide sequence that hybridizes under stringent conditions to the nucleotide sequence of SEQ ID NO: 1-37, aids in diagnosis of COVID-19. Such nucleotides include, but are not limited to probes, primers, controls, and that specifically binds to the nucleotide foci of the invention.

TABLE 1
SARS-Cov-2 genome structure and commercialized RTPCR kit primer and probe [A-1]
location(reference: Wuhan seafood market pneumonia virus isolate Wuhan-Hu-1, complete
genome GenBank: MN908947.3), compared with primer/probes of the kit of the systems
and methods herein, where the YES designation indicates a determined genome marker.

| | | Location of Primer/Probe in commercial |
|---|---|---|
| Region | Domain, Protein (bp length) | COVID-19 RT-PCR kit including GG |
| ORF | | |
| ORF1a | 1-13218 | |
| | Leader protein (1-540), | |
| | Nsp2 (541-2454) | |
| | Nsp3 (2455-8289) | |
| | Nsp4 (8290-9789) | |
| | 3c-like protease (9790-10707) | |
| | Nsp6 (10708-11577) | |
| | Nsp 7 (11578-11826) | |
| | Nsp 8 (11827-12420) | |
| | Nsp 9 (12421-12757) | |
| | Nsp 10 (12760-13176) | |
| ORF1ab | RNA-dependent RNA polymerase | YES (popular in many kits, recommended by |
| | (RdRp) (13177-13203, 13203-15971) | WHO). Primers and Probe of ORF1ab gene in |
| | | GG COVID-19 kit is located in from 13348 |
| | | to 13452 of ORF1ab and RdRp in 15441- |
| | | 15226 whereas the most popular region of |
| | | primers of RdRp is from 15361-15460* |
| | Helicase (NTPase/herlicase domain) | YES |
| | (15972-17774) | |
| | 3-to-5 exonuclease (17775-19395) | |
| | EndoRNAase( 19356-20393) | |
| | 2-O-ribosemethyltransferase (20394-21297) | |
| Structural | Spike, S (21563-25384) | YES |
| protein | Envelope, E (26245-26472) | YES |
| | M (26523-27191) | |
| | Nucleocapid protein, N (28274-29903) | YES (kits from CDC of USA, popular in |
| | | many kits). Primers and Probe of N gene in |
| | | GG COVID-19 kit is located in from 28881- |
| | | 28971(N1) 28863-28980 (N2), whereas most |
| | | popular region of primers of RdRp is in from |
| | | 28555 to 28,682*. |

*Reference:
1) Corman VR et als. Detection of 2019 novel coronavirus (2019-nCoV) by real-time RT-PCR. Euro Surveill. 2020 January; 25(3). doi: 10.2807/1560-7917.ES.2020.25.3.2000045
2) CDC 2019-Novel Coronavirus (2019-nCov) real time RTPCR Diagnositc Panel. Catalog #2019-nCoVEUA-01. CDC, Division of Viral Disease. Atlanta, GA, USA.

TABLE 2
Primers and Probes for RT-Real TIME PCR Assay Reported by Literature [A-1] for Orders
A and B of Table 3 below.

| | Journal title | Author | primer/probe |
|---|---|---|---|
| 1 | Molecular Diagnosis of a | Daniel | N gene assay are: 5′- |
| | Novel Coronavirus (2019- | K. W. et al. | TAATCAGACAAGGAACTGATTA-3 (Forward), 5′- |
| | nCoV) Causing an Outbreak | | CGAAGGTGTGACTTCCATG-3 (Reverse) and 5′- |
| | of Pneumonia | | GCAAATTGTGCAATTTGCGG-3 (Probe in 5′- |
| | | | FAM/ZEN/3′-IBFQ format) |
| | | | ORF1b gene assay are: 5′- |
| | | | TGGGGYTTTACRGGTAACCT-3′(Forward; |
| | | | Y¼C/T, R¼A/G), 5′- |
| | | | AACRCGCTTAACAAAGCACTC-3′(Reverse; |
| | | | R¼A/G) |
| | | | and 5′-(Probe in 5′-FAM/ZEN/3′-IBFQ format; |
| | | | W¼A/T)TAGTTGTGATGCWATCATGACTAG-3 |
| 2 | Rapid and visual detection of | C. Yan et | orf1ab-F: 5′-CAGACCTCGTCTATGCTTTAAGGC-3′; |
| | 2019 novel coronavirus | als. 1 | orf1ab-R: 5′-CCCTGGTCAAGGTTAATATAGGCA-3′ |
| | (SARS-CoV-2) by a reverse | | |
| | transcription loop-mediated | | |
| | isothermal amplification | | |
| | assay | | |
| 3 | Research Use Only 2019- | CDC | 2019-nCoV_N1-F; 5′-GAC CCC AAA ATC AGC |
| | Novel Coronavirus (2019- | | GAA AT-3′, 2019-nCoV_N1-R; 5′-TCT GGT TAC |
| | nCoV) Real-time RT-PCR | | TGC CAG TTG AAT CTG-3′, 2019-nCoV_N1-P; 5′- |
| | Primer and Probe | | FAM-ACC CCG CAT TAC GTT TGG TGG ACC- |
| | Information | | BHQ1-3′, 2019-nCoV_N2-F; 5′-TTA CAA ACA TTG |
| | (https://www.cdc.gov/corona | | GCC GCA AA-3′, 2019-nCoV_N2-R; 5′-GCG CGA |
| | virus/2019-ncov/lab/rt-per- | | CAT TCC GAA GAA-3′, 2019-nCoV_N2-P; 5′-FAM- |
| | panel-primer-probes.html) | | ACA ATT TGC CCC CAG CGC TTC AG-BHQ1-3′, |
| | | | 2019-nCoV_N3-F; 5′-GGG AGC CTT GAA TAC |
| | | | ACC AAA A-3′, 2019-nCoV_N3-R; 5′-TGT AGC |
| | | | ACG ATT GCA GCA TTG-3′, 2019-nCoV_N3-P; 5′- |
| | | | FAM-AYC ACA TTG GCA CCC GCA ATC CTG- |
| | | | BHQ1-3′, RP-F; 5′-AGA TTT GGA CCT GCG AGC |
| | | | G-3′, RP-R; 5′-GAG CGG CTG TCT CCA CAA GT- |
| | | | 3′, RP-P; 5′-FAM - TTC TGA CCT GAA GGC TCT |
| | | | GCG CG - BHQ-1-3′ |
| 4CDC | rRT-PCR Primers and Probes | CDC | 2019-nCoV_N1-F; GAC CCC AAA ATC AGC GAA |
| | for nCoV Coronavirus | | AT, 2019-nCoV_N1-R; TCT GGT TAC TGC CAG |
| | (https://eurofinsgenomics.eu/ | | TTG AATCTG, 2019-nCoV_N1-Probe; FAM-ACC |
| | en/dna-rna- | | CCG CAT TAC GTT TGG TGG ACC-BHQ1, 2019- |
| | oligonucleotides/optimised- | | nCoV_N2-F; TTA CAA ACA TTG GCC GCA AA, |
| | application-oligos/ncov-qpcr- | | 2019-nCoV_N2-R; GCG CGA CAT TCC GAA GAA, |
| | assays/) | | 2019-nCoV_N2-probe; FAM-ACA ATT TGC CCC |
| | | | CAG CGC TTC AG-BHQ1, 2019-nCoV_N3-F; GGG |
| | | | AGC CTT GAA TAC ACC AAA A, 2019-nCoV N3- |
| | | | R; TGT AGC ACG ATT GCA GCA TTG, 2019- |
| | | | nCoV N3-Probe; FAM-AYC ACA TTG GCA CCC |
| | | | GCA ATC CTG-BHQ1, RNAse P-F; AGA TTT GGA |
| | | | CCT GCG AGC G, RNAse P-R; GAG CGG CTG |
| | | | TCT CCA CAA G, RNAse P-Probe; FAM - TTC |
| | | | TGA CCT GAA GGC TCT GCG CG - BHQ-1 |
| 5-WHO | | WHO | RdRP_SARSr- |
| | | | F2; GTGARATGGTCATGTGTGGCGG, |
| | | | RdRP_SARSr- |
| | | | R1; CARATGTTAAASACACTATTAGCATA, |
| | | | RdRP_SARSr-RdRP_SARSr- |
| | | | P1; FAMCCAGGTGGWACRTCATCMGGTGATGC |
| | | | BBQP2; FAMCAGGTGGAACCTCATCAGGAGAT |
| | | | GCBBQ, |
| | | | E_Sarbeco_F1; ACAGGTACGTTAATAGTTAATAG |
| | | | CGT, |
| | | | E_Sarbeco_R2; ATATTGCAGCAGTACGCACACA, |
| | | | E_Sarbeco_R2; ATATTGCAGCAGTACGCACACA, |
| | | | E_Sarbeco_P1; FAMACACTAGCCATCCTTACTGC |
| | | | GCTTCGBBQ |
| 5 | Development of a reverse | Yun Hee | Synthesized SARS-CoV-2 N geneA- |
| | transcription-loop mediated | Baek et als. | F; TAATACGACTCACTATAGGGTATCATGACGT |
| | isothermal amplification as a | | TCGTGT, SARS-CoV-2 N geneB- |
| | rapid early-detection method | | R; GGGCCAGTTCCTAGGTAA |
| | for novel SARS-CoV-2 | | TGTTTTA, R; GGCCCAGTTCCTAGGTAGTA, |
| | | | Synthesized SARS-CoV-2 N geneB- |
| | | | F; TAATACGACTCACTATAGGGGCATTTAGAG |
| | | | ACGTACT |
| | | | TGTTGT, R; GGGCCAGTTCCTAGGTAA |

TABLE 3
Steps of invention and the examples related to each step

| Order | Project and invention | Specifics | Example |
|---|---|---|---|
| A | Preparation for the systems | | |
| | and methods herein | | |
| A-1 | Articles, research, planning | choose SARS-Cov-2 total genomic RNA | |
| | | (MN908947.3) | |
| B | Preparation of constituents of | primer, probe, control prep | |
| | the systems and methods herein | | |
| B-1 | target gene primer, probe | SARS-Cov-2-N-1/2, ORF1ab, RdRp gene | 1-4 |
| | selection | marker/internal control- beta-actin marker | |
| | | Blast analysis Tm analysis −>decide on | |
| | | primer/probe | |
| B-2 | Primer and probe production | Order primer/probe, produce, dilute ordered | 1-4 |
| | | primer/probe | |
| B-3 | Internal control and Positive | Internal control (IC) RT-PCR and in vitro | 5.1 |
| | control | transcript plasmid RNA | 5.2 |
| | | Positive control: SARS-Cov-2 total | 6 |
| | | genomic RNA Twist RNA (SARS-Cov-2; | |
| | | MT007544.1 and MN908947.3 Genbank; | |
| | | Twist Bioscience, San Francisco, CA , | |
| | | USA) dilution (10\hat{ }6−>10\hat{ }2 copies/ul) | |
| C | Real Time RT-PCR of the | Decide on Real-time RT-PCR specifics | |
| | systems and methods herein | | |
| C-1 | Reagent constituent | One-step PCR Premix (reverse | 7 |
| | | transcripton/real time PCR in 1 tube, 1 step) | |
| C-2 | PCR steps and temperature/time | Decide steps/time by individual gene | 8 |
| | | marker characteristic, reverse transcriptase, | |
| | | Taq polymerase finalize PCR conditions | |
| C-3 | Application test of the systems | Perform Realtime RT PCR with Standard | |
| | and methods herein | material evaluate results | |
| | C-3.1 | Nand IC duplex Real-Time RT PCR | 9.1 |
| | C.3.2 | ORF1ab and IC duplex Real-Time RT PCR | 9.2 |
| | C.3.3 | RdRp and IC duplex Real-Time RT PCR | 9.3 |
| | C.3.4 | N and or f1ab and IC triplex-1 Real-Time | 10 |
| | | RT PCR | |
| | C.3.5 | RdRp and Orf1ab and IC triplex-2 Real- | 11 |
| | | Time RT PCR | |
| D | Final products of the systems | (COVID-19 Quadplex RT-real time PCR | 12-13 |
| | and methods herein | kit) final invention, Repeated LOD/cutoff | |
| | | formulation, result conclusion | |
| D-1 | reagent optimization | Goodgene Premix prep | 12 |
| D-2 | PCR step optimization | Real-Time RT PCR repeat, optimize | 12 |
| D-3 | Finalize SOP steps | | 12 |
| D-4 | Finalize result conclusion | | 13 |
| E | Performance test for final | Preparation of Materials for Validation | |
| | invention | | |
| E-1 | limit of detection (LoD) | | 14 |
| E-2 | analytic sensitivity | | 15 |
| E-3 | Inclusivity | | 16 |
| E-4 | specificity and cross reactivity | | 17 |
| E-5 | Interference | | 18 |
| E-6 | Claiming Multiple Instruments | | 19 |
| E-7 | Precision (repeatability and | | 20 |
| | reproducibility) | | |
| E-8 | Stability and storage conditions | | 21 |
| E-9 | Clinical performance with BEI | | 22 |
| | in clinical matrix | | |
| E-10 | Clinical performance with | | 23 |
| | clinical specimens in | | |
| | comparison with Seegene | | |

TABLE 4
Target Gene for Reverse Transcription-Real Time PCR Assay of SARS-Cov-2

| | | | Primer and Probe | Tagged | Possible |
|---|---|---|---|---|---|
| | Name of Target Gene | Role | sequence | Probe | Probe |
| 1 | N of SARS-Cov-2 (N-2) | Gene to detect SARS-Cov-2 | SEQ 1, 2, and 10 | 11 | 9 |
| 2 | ORF1ab of SARS-Cov-2 | Gene to detect SARS-Cov-2 | SEQ 3, 4 and 13 | 14 | 12 |
| 3 | RdRp* of SARS-Cov-2 | Gene to detect SARS-Cov-2 | SEQ 5, 6 and 16 | 17 | 15 |
| 4 | Beta-actin of human | IC** gene to avoid false | SEQ 7, 8 and 18 | 19 | — |
| | | positive result | | | |
| 5 | N of SARS-Cov-2 (N-1) | Gene to detect SARS-Cov-2 | SEQ 20, 21 and 23 | 24 | 22 |
| 6 | PBGD** of human | IC gene to avoid false | SEQ 25, 26 and 27 | 28 | — |
| | | positive result | | | |

(Abbreviation)
RdRp*: RNA dependent RNA polymerase
IC**: Internal control marker; human housekeeping gene
PBGD***: porphobilinogen deaminase

TABLE 5
Fluorescent labeled probe of each marker and examples of utilization/application
in the systems and methods herein.

| | Fluoresecent | | | |
|---|---|---|---|---|
| Name of Target Gene | probe ID | Reporter | Quencher | Application |
| RdRp* of SARS-Cov-2 | 17 | FAM | BHQ1 | Most other PCR* excluding Tri-1 |
| ORF1ab of SARS-Cov-2 | 14 | FAM | BHQ1 | Tri-1 |
| | 14~2 | Cy-5 | BHQ2 | Most other PCR |
| N of SARS-Cov-2 (N-1) | 24 | Texas Red | BHQ2 | Triplex-1 |
| N of SARS-Cov-2 (N-2) | 11 | Texas Red | BHQ2 | Quadplex, Triplex-2, Duplex-1/2/3/4 |
| Beta-actin of human | 19 | Hex | BHQ1 | Most other PCR |
| PBGD** of human | 28 | Hex/Vic | BHQ1 | Tri-1 |

In the tables below, the single and multiplex PCR comprised of above constituents are disclosed.

TABLE 6
Triplex-1

| | | | | | | | NCBI |
|---|---|---|---|---|---|---|---|
| Seq | | seq | | | | | Accession |
| ID | Target | ID | oligo | Sequences (5′==>3′) | Amplicon | nt site | No. |
| 5 | N-1 | 5 | COV_N2F | 5′-ggggaacttctcctgctagaat-3′ | 91 bp | 28881-28971 | MN908947.3 |
| 6 | | 6 | COV_N2R | 5′-ttgctctcaagctggttcaa-3′ | | | |
| 11 | | 11 | COV_NP | 5′-TexasRed- | TR-5′- | | |
| | | | | ttgctgctgcttgacagatt-BHQ2-3′ | 3′BHQ2 | | |
| 21 | 28861 ttcaactcca ggcagcagta ggggaacttc tcctgctaga atggctggca atggcggtga | | | | | | |
| | 28921 tgctgctctt gctttgctgc tgcttgacag attgaaccag cttgagagca aaatgtctgg | | | | | | |
| 3 | ORF1ab | 3 | COV_ORF_2F | gggttttacacttaaaaacacagtc | 105 bp | 13348-13452 | MN908947.3 |
| 4 | | 4 | COV_ORF_2R | Gcatcagctgactgaagcat | | | |
| 10 | | 10 | COV_ORF_P | Ccgtctgcggtatgtggaaaggttat | CY5-5′- | | |
| | | | | gg | 3′BHQ1 | | |
| 20 | 13321 acctacaact tgtgctaatg accctgtggg ttttacactt aaaaacacag tctgtaccgt | | | | | | |
| | 13381 ctgcggtatg tggaaaggtt atggctgtag ttgtgatcaa ctccgcgaac ccatgcttca | | | | | | |
| | 13441 gtcagctgat gcacaatcgt ttttaaacgg gtttgcggtg taagtgcagc ccgtcttaca | | | | | | |
| 15 | Pbgd | 15 | PBGDF | 5′-gagaccaggagtcagactgt-3′ | 127 bp | 21-147 | NM_000190.4 |
| 16 | | 16 | PBGDR | 5′-gcttggaaagtaggctgtgt-3′ | | | |
| 17 | | 17 | PBGDP | 5′-HEX-cacgtgtccccggtactc- | 5′-HEX- | | |
| | | | | BHQ1-3′ | 3′-BHQ1 | | |

| | |
|---|---|
| 24 | 1 aagtgacgcg aggctctgcg gagaccagga gtcagactgt aggacgacct cgggtcccac |
| | 61 gtgtccccgg tactcgccgg ccggagcccc cggcttcccg gggccggggg accttagcgg |
| | 121 cacccacaca cagcctactt tccaagcgga gccatgtctg gtaacggcaa tgcggctgca |

TABLE 7
Triplex-2:

| | | | | | | NCBI |
|---|---|---|---|---|---|---|
| seq | | | | | | Accession |
| ID | Target | Oligo | Sequences (5′==>3′) | Amplicon | nt site | No. |
| 1 | RdRp | RD1F | catgtgtggcggttcactat | 86 bp | 15441~15526 | MN908947.3 |
| 2 | | RD4R | tgttaaaaacactattagcataagcag | | | |
| 9 | | RdRP_SARS | caggtggaacctcatcaggagatgc | FAM/BHQ1 | WHO | SARS |
| | | r-P2 | | | PROBE_P2 | COV2 |
| | | | | | | Specific |
| 19 | 15421 caagtattga gtgaaatggt catgtgtggc ggttcactat atgttaaacc aggtggaacc | | | | | |
| | 15481 tcatcaggag atgccacaac tgcttatgct aatagtgttt ttaacatttg /tcaagctgtc | | | | | |
| 7 | N-2 | N5F | caactccaggcagcagtagg | 118 bp | 28863-28980 | MN908947.3 |
| 8 | | N1R | ccagacattttgctctcaagc | | | |
| 12 | | COV_NP | TTGCTGCTGCTTGACAGA | TR-5′- | | |
| | | | TT | 3′BHQ2 | | |
| 22 | 28861 ttcaactcca ggcagcagta ggggaacttc tcctgctaga atggctggca atggcggtga | | | | | |
| | 28921 tgctgctctt gctttgctgc tgcttgacag attgaaccag cttgagagca aaatgtctgg | | | | | |
| 13 | beta- | ACTF | GCA CCA CAC CTT CTA | 102 bp | 342~443 | NM_001101.5 |
| | Actin | | CAA TGA | | | |
| 14 | | ACTR | GTC ATC TTC TCG CGG | | | |
| | | | TTG GC | | | |
| 18 | | ACTP | CAC CCC GTG CTG CTG | 5′-HEX-3′- | | |
| | | | ACC GAG GC | BHQ1 | | |

| | |
|---|---|
| 23 | 301 cacggcatcg tcaccaactg ggacgacatg gagaaaatct ggcaccacac cttctacaat |
| | 361 gagctgcgtg tggctcccga ggagcacccc gtgctgctga ccgaggcccc cctgaacccc |
| | 421 aaggccaacc gcgagaagat gacccagatc atgtttgaga ccttcaacac cccagccatg |

TABLE 8
QPlex

| | | | | | | NCBI |
|---|---|---|---|---|---|---|
| seq | | | | | | Accession |
| ID | Target | Oligo | Sequences (5′==>3′) | Amplicon | nt site | No. |
| 1 | RdRp | RD1F | catgtgtggcggttcactat | 86 bp | 15441~15526 | MN908947.3 |
| 2 | | RD4R | tgttaaaaacactattagcataagcag | | | |
| 9 | | RdRP_SAR | CAGGTGGAACCTCATCAGGAG | FAM/BHQ1 | WHO | SARS |
| | | Sr-P2 | ATGC | | PROBE_P2 | COV2 |
| | | | | | | Specific |
| 19 | 15421 caagtattga gtgaaatggt catgtgtggc ggttcactat atgttaaacc aggtggaacc | | | | | |
| | 15481 tcatcaggag atgccacaac tgcttatgct aatagtgttt ttaacatttg /tcaagctgtc | | | | | |
| 7 | N-2 | N5F | caactccaggcagcagtagg | 118 bp | 28863-28980 | MN908947.3 |
| 8 | | N1R | ccagacattttgctctcaagc | | | |
| 12 | | COV_NP | TTGCTGCTGCTTGACAGATT | TR-5′- | | |
| | | | | 3′BHQ2 | | |
| 22 | 28861 ttcaactcca ggcagcagta ggggaacttc tcctgctaga atggctggca atggcggtga | | | | | |
| | 28921 tgctgctctt gctttgctgc tgcttgacag attgaaccag cttgagagca aaatgtctgg | | | | | |
| 3 | ORF1ab | COV_ORF_2F | gggttttacacttaaaaacacagtc | 105 bp | 13348-13452 | MN908947.3 |
| 4 | | COV_ORF_2R | gcatcagctgactgaagcat | | | |
| 10 | | COV_ORF_P | CCGTCTGCGGTATGTGGAAAG | CY5-5′- | | |
| | | | GTTATGG | 3′BHQ2 | | |
| 20 | 13321 acctacaact tgtgctaatg accctgtggg ttttacactt aaaaacacag tctgtaccgt | | | | | |
| | 13381 ctgcggtatg tggaaaggtt atggctgtag ttgtgatcaa ctccgcgaac ccatgcttca | | | | | |
| | 13441 gtcagctgat gcacaatcgt ttttaaacgg gtttgcggtg taagtgcagc ccgtcttaca | | | | | |
| 13 | beta- | ACTF | GCA CCA CAC CTT CTA CAA | 102 bp | 342~443 | NM_001101.5 |
| | Actin | | TGA | | | |
| 14 | | ACTR | GTC ATC TTC TCG CGG TTG GC | | | |
| 18 | | ACTP | CAC CCC GTG CTG CTG ACC | 5′-HEX- | | |
| | | | GAG GC | 3′-BHQ1 | | |

| | |
|---|---|
| 23 | 301 cacggcatcg tcaccaactg ggacgacatg gagaaaatct ggcaccacac cttctacaat |
| | 361 gagctgcgtg tggctcccga ggagcacccc gtgctgctga ccgaggcccc cctgaacccc |
| | 421 aaggccaacc gcgagaagat gacccagatc atgtttgaga ccttcaacac cccagccatg |

TABLE 9
Duplexes

| | | | | | |
|---|---|---|---|---|---|
| | | | | | NCBI |
| | | | | | Accession |
| Target | Oligo | Sequences (5′==>3′) | Amplicon | nt site | No. |

Rdrp/ic (beta actin) Duplex-1

| | | | | | |
|---|---|---|---|---|---|
| RdRp | RD1F | 5′-CATGTGTGGCGGTTCACTAT-3′ | 86 bp | 15441~15526 | MN908947.3 |
| | RD4R | 5′-TGTTAAAAACACTATTAGCATAAGC | | | |
| | | AG-3′ | | | |
| | RdRP_SARSr- | 5′-FAM- | FAM/BHQ1 | WHO | SARS |
| | P2 | CAGGTGGAACCTCATCAGGAGATG | | PROBE_P2 | COV2 |
| | | C-BHQ1-3′ | | | Specific |

15421 caagtattga gtgaaatggt catgtgtggc ggttcactat atgttaaacc aggtggaacc
15481 tcatcaggag atgccacaac tgcttatgct aatagtgttt ttaacatttg /tcaagctgtc

| | | | | | |
|---|---|---|---|---|---|
| beta- | ACTF | 5′-GCACCACACCTTCTACAATGA-3′ | 102 bp | 342~443 | NM_001101.5 |
| Actin | ACTR | 5′-GTCATCTTCTCGCGGTTGGC-3′ | | | |
| | ACTP | 5′-HEX-CAC CCC GTG CTG CTG | 5′-HEX-3′- | | |
| | | ACC GAG GC-BHQ1-3′ | BHQ1 | | |

301 cacggcatcg tcaccaactg ggacgacatg gagaaaatct ggcaccacac cttctacaat
361 gagctgcgtg tggctcccga ggagcacccc gtgctgctga ccgaggcccc cctgaacccc
421 aaggccaacc gcgagaagat gacccagatc atgtttgaga ccttcaacac cccagccatg
Duplex-2

| | | | | | |
|---|---|---|---|---|---|
| ORF1ab | COV_ORF_2F | Gggttttacacttaaaaacacagtc | 105 bp | 13348-13452 | MN908947.3 |
| | COV_ORF_2R | Gcatcagctgactgaagcat | | | |
| | COV_ORF_P | CCGTCTGCGGTATGTGGAAAGGTTA | CY5-5′- | | |
| | | TGG | 3′BHQ1 | | |

13321 acctacaact tgtgctaatg accctgtggg ttttacactt aaaaacacag tctgtaccgt
13381 ctgcggtatg tggaaaggtt atggctgtag ttgtgatcaa ctccgcgaac ccatgcttca
13441 gtcagctgat gcacaatcgt ttttaaacgg gtttgcggtg taagtgcagc ccgtcttaca

| | | | | | |
|---|---|---|---|---|---|
| beta- | ACTF | GCA CCA CAC CTT CTA CAA TGA | 102 bp | 342~443 | NM_001101.5 |
| Actin | ACTR | GTC ATC TTC TCG CGG TTG GC | | | |
| | ACTP | CAC CCC GTG CTG CTG ACC GAG | 5′-HEX-3′- | | |
| | | GC | BHQ1 | | |

301 cacggcatcg tcaccaactg ggacgacatg gagaaaatct ggcaccacac cttctacaat
361 gagctgcgtg tggctcccga ggagcacccc gtgctgctga ccgaggcccc cctgaacccc
421 aaggccaacc gcgagaagat gacccagatc atgtttgaga ccttcaacac cccagccatg
Duplex-3 N-2/IC

| | | | | | |
|---|---|---|---|---|---|
| N-2 | N5F | Caactccaggcagcagtagg | 118 bp | 28863-28980 | MN908947.3 |
| | N1R | Ccagacattttgctctcaagc | | | |
| | COV_NP | Ttgctgctgcttgacagatt | TR-5′- | | |
| | | | 3′BHQ2 | | |

28861 ttcaactcca ggcagcagta ggggaacttc tcctgctaga atggctggca atggcggtga
28921 tgctgctctt gctttgctgc tgcttgacag attgaaccag cttgagagca aaatgtctgg

| | | | | | |
|---|---|---|---|---|---|
| beta- | ACTF | GCA CCA CAC CTT CTA CAA TGA | 102 bp | 342-443 | NM_001101.5 |
| Actin | ACTR | GTC ATC TTC TCG CGG TTG GC | | | |
| | ACTP | CAC CCC GTG CTG CTG ACC GAG | 5′-HEX-3′- | | |
| | | GC | BHQ1 | | |

301 cacggcatcg tcaccaactg ggacgacatg gagaaaatct ggcaccacac cttctacaat
361 gagctgcgtg tggctcccga ggagcacccc gtgctgctga ccgaggcccc cctgaacccc
421 aaggccaacc gcgagaagat gacccagatc atgtttgaga ccttcaacac cccagccatg

| | | | | | |
|---|---|---|---|---|---|
| N-1 | COV_N1F | 5′-CAACTCCAGGCAGCAGTAGG-3′ | 91 bp | 28881-28971 | MN908947.3 |
| | COV_N5R | 5′-CCAGACATT1TGCTCTCAAGC-3′ | | | |
| | COV_NP | 5′-TexasRed- | TR-5′- | | |
| | | TTGCTGCTGCTTGACAGATT-BHQ2-3′ | 3′BHQ2 | | |

28861 TTCAACTCCA GGCAGCAGTA GGGGAACTTC TCCTGCTAGA ATGGCTGGCA
ATGGCGGTGA
28921 TGCTGCTCTT GCTTTGCTGC TGCTTGACAG ATTGAACCAG CTTGAGAGCA
AAATGTCTGG

| | | | | | |
|---|---|---|---|---|---|
| beta- | ACTF | GCA CCA CAC CTT CTA CAA TGA | 102 bp | 342~443 | NM_001101.5 |
| Actin | ACTR | GTC ATC TTC TCG CGG TTG GC | | | |
| | ACTP | CAC CCC GTG CTG CTG ACC GAG | 5′-HEX-3′- | | |
| | | GC | BHQ1 | | |

301 cacggcatcg tcaccaactg ggacgacatg gagaaaatct ggcaccacac cttctacaat
361 gagctgcgtg tggctcccga ggagcacccc gtgctgctga ccgaggcccc cctgaacccc
421 aaggccaacc gcgagaagat gacccagatc atgtttgaga ccttcaacac cccagccatg

TABLE 10
Single PCR

| | | | | | NCBI |
|---|---|---|---|---|---|
| Target | Oligo | Sequences (5′==>3′) | Amplicon | nt site | Accession No. |
| RdRp | RD1F | 5′-CATGTGTGGCGGTTCACTAT-3′ | 86 bp | 1544115526 | MN908947.3 |
| | RD4R | 5′-TGTTAAAAACACTATTAGCATAAG | | | |
| | | CAG-3′ | | | |
| | RdRP_SARSr- | 5′-FAM- | FAM/ | WHO | SARS COV2 |
| | P2 | CAGGTGGAACCTCATCAGGAGAT | BHQ1 | PROBE_P2 | Specific |
| | | GC-BHQ1-3′ | | | |

15421 caagtattga gtgaaatggt catgtgtggc ggttcactat atgttaaacc aggtggaacc
15481 tcatcaggag atgccacaac tgcttatgct aatagtgttt ttaacatttg /tcaagctgtc

TABLE 11
Internal control-1(Seq ID No: 29; beta actin)
1. Control RNA(Internal Control/QPlex)

| | Genebank | |
|---|---|---|
| Seq no. | location | Base sequence |
| | NM_001101.5 | 5′-CACGGCATCG TCACCAACTG GGACGACATG GAGAAAATCT |
| | | GGCACCACAC CTTCTACAAT GAGCTGCGTG TGGCTCCCGA |
| | | GGAGCACCCC GTGCTGCTGA CCGAGGCCCC CCTGAACCCC |
| | | AAGGCCAACC GCGAGAAGAT GACCCAGATC ATGTTTGAGA |
| | | CCTTCAACAC CCCAGCCATG-3′ |

TABLE 12
Internal control-2 (Pbgd)
PBGD RNA(Internal Control)

| | | Length | Genebank | |
|---|---|---|---|---|
| Seq No. | plasmid | (mer) | location | Base sequence |
| 22 | PBGD | 127 | NG_008093 | 5′- |
| | (HMBS) | | | GAGACCAGGAGTCAGACTGTAGGACGACCTCG |
| | | | | GGTCCCACGTGTCCCCGGTACTCGCCGGCCGG |
| | | | | AGCCCCCGGCTTCCCGGGGCCGGGGGACCTTA |
| | | | | GCGGCACCCACACACAGCCTAC1TTCCAAGC- |
| | | | | 3′ |

The systems and methods herein include: (i) multi-aligned the base sequences of these coronaviruses; and (ii) designed primers and probes out of the: (a) ORFlab (Open Reading Frame lab), (b) RdRP (RNA-dependent RNA polymerase) and (c) virus nucleoprotein(N) gene. The primers and probes of N-1, N-2, Orflab, RdRP have not been reported to the knowledge of inventors and has no significant homology with known respiratory pathogens, human genome, or other coronaviruses (100% specificity), and include all reported 1500 SARS-Cov-2 strains to date (Apr. 16, 2020). Also this particular combination of N, Orflab and RdRP genes with human beta actin/Pbgd genes as internal controls have not been reported to date to the knowledge of the inventors herein. (See Table 2).

The systems and methods herein include: (i) a real-time RT-PCR test method used for (ii) the qualitative detection of nucleic acid from the 2019-nCoV in upper and lower respiratory specimens (e.g., nasopharyngeal or oropharyngeal swabs, sputum, lower respiratory tract aspirates, bronchoalveolar lavage, and nasopharyngeal wash/aspirate or nasal aspirate), as (iii) collected from individuals who meet 2019-nCoV clinical and/or epidemiological criteria (e.g., clinical signs and symptoms associated with 2019-nCoV infection, contact with a probable or confirmed 2019-nCoV case, history of travel to geographic locations where 2019-nCoV cases are frequently detected, or other epidemiologic links for which 2019-nCoV testing may be indicated as part of a public health investigation).

In the systems and methods herein, the results obtained from the real-time RT-PCR are for the identification of SARS-CoV-2 RNA. SARS-CoV-2 RNA is generally detectable in human nasopharyngeal swab and nasopharyngeal swab specimens during the acute phase of infection. Positive results are indicative of presence of SARS-CoV-2 RNA but do not rule out bacterial infection or co-infection with other viruses. Clinical correlation with patient history and other diagnostic information is necessary to determine patient infection status. Positive results do not rule out bacterial infection or co-infection with other viruses. Laboratories within the United States and its territories are required to report all positive results to the appropriate public health authorities. Negative results do not preclude SARS-CoV-2 infection and should not be used as the sole basis for patient management decisions. Negative results must be combined with clinical observations, patient history, and epidemiological information.

In the systems and methods herein, the fluorescent probe binds to the target DNA between the two unlabeled PCR primers. During the extension period of the PCR, Taq polymerase extends the unlabeled primers using the template strand as a guide; when it reaches the probe it cleaves the probe separating the reporter dye from the quencher dye, resulting in a fluorescent signal. The real-time PCR instrument detects this fluorescence intensity from the unquenched dye. With each cycle of PCR, more probes are cleaved resulting in an increase in fluorescence. Fluorescence intensity is monitored at each PCR cycle in corresponding channels at a threshold of 0.05.

### Tri-1/2 and QPlex Primers

In the systems and methods herein, preferred primers to be used in a RT-PCR method are listed in Table 1, in the presence of MgCl2 and the thermal cycles are, for example, but not limited to, 42° C. for 3 min, 95° C. for 10 minutes, and followed by 40 cycles of 95° C. for 15 seconds, 56° C. for 40 seconds. In the systems and methods herein, these multiplex Real time PCRs are two or all of the following thee-nucleic acid containing combinations: (Combination 1) SEQ ID NO 1,2,10/11; (Combination 2) SEQ ID NO: 3,4,13/14; (Combination 3) SEQ ID NO: 5,6,18/19; (Combination 4 for COVID markers) SEQ ID NO: 20,21,23/24; and one of: (Combinations 5 for human internal control) Seq ID NO: 7,8,18/19 and (Combination 5 for human control) Seq ID NO: 25, 26, 27/28.

### Duplex and Single Primers

In the systems and methods herein, preferred primers used in a RT-PCR method are in the presence of MgCl2. The thermal cycles are, for example, but not limited to, 50° C. for 30 min, 95° C. for 10 minutes, and followed by 40 cycles of 95° C. for 15 seconds, 56° C. for 40 seconds, and a final hold at 40° C. for 10 seconds. In the systems and methods herein, these multiplex Real time PCRs are a combination of one of: (Combination 1) SEQ ID NO: 1,2,10/11; (Combination 2) SEQ ID NO: 3,4,13/14; (Combination 3) Seq ID NO 5,6,8/19; (Combination 4 for COVID markers) Seq ID NO: 20,21,23/24, with or without Seq ID NO: 7,8,18/19 for human internal control.

The systems and methods herein involve a real-time qualitative PCR assay. For example, the qualitative PCR used in the present invention is TaqMan® assay (Holland et al., Proc Natl Acad Sci USA 88(16):7276 (1991). The assays can be performed on an instrument designed to perform such assays, for example those available from Applied Biosystems (Foster City, Calif.) or Rotorgene Q (Rotorgene, Qiagene, Hilden, Germany).

In the systems and methods herein, a real-time qualitative PCR assay is provided to detect the presence of the COVID-19 virus, natural or artificial variants, analogs, or derivatives thereof, in a biological sample by subjecting the cDNA obtained by reverse tran-scription of the extracted total RNA from the sample to PCR reactions using specific primers, and detecting the amplified product using a probe.

### Tri and QPlex Probes

In the systems and methods herein, for Triplex 1,2/QPlex/Duplex, the probe is a TaqMan® probe comprising an oligonucleotide with a 5′-reporter dye and a 3′-quencher dye. The fluores-cence signals from these reactions are captured at the end of extension steps as PCR product is generated over a range of the thermal cycles, thereby allowing the quantitative determination of the viral load in the sample based on an amplification plot.

Other techniques for detection of RNA may be used. For example, in vitro techniques for detection of mRNA include northern hybridizations, in situ hybridizations, DNA microarrays, RT-PCR, and RNase protection. In vitro techniques for detection of genomic RNA include northern hybridizations, RNA microarrays, RT-PCT, and RNase protection.

As discussed above, the polynucleotides of the Sars-CoV-2 virus may be amplified before they are detected. The term “amplified” refers to the process of making multiple copies of the nucleic acid from a single polynucleotide molecule. The amplification of polynucle-otides can be carried out in vitro by biochemical processes known to those of skill in the art. The amplification agent may be any compound or system that will function to accomplish the synthesis of primer extension products, including enzymes. Suitable enzymes for this purpose include, for example, E. coli DNA polymerase I, Taq poly-merase, Kienow fragment of E. coli DNA polymerase I, T4 DNA polymerase, other available DNA polymerases, poly-merase muteins, reverse transcriptase, ligase, and other

### Triplex-1

In the systems and methods herein, the N-2 probe has a nucleotide sequence of 5′-3′ (SEQ ID NO: 11) for ORFlab and nucleocapsid (N) gene. The TaqMan probes for the two amplicons are labeled with FAM and Texas red fluorescent dyes, respectively, to generate target-specific signal. The assay includes an RNA internal control (PBGD; Porphobilinogen deaminase) to monitor the processes from nucleic acid extraction to fluorescence detection. The IC probe is labeled with Hex (VIC) fluorescent dye to differentiate its fluorescent signal from SARS-CoV-2 targets.

### QPlex (QuadPlex)

In the systems and methods herein, the probe has a nucleotide sequence of 5′-3′ (SEQ ID NO: 14). For the RdRP specific probe, the signal from the fluorescent dye (FAM) on the 5′ end is quenched by BHQ-1 on its 3′ end. For the N specific probe, the signal from the fluorescent dye (Texas Red) on the 5′ end is quenched by BHQ-2 on its 3′ end. For the Orflab specific probe, the signal from the fluorescent dye (Cy5) on the 5′ end is quenched by BHQ-2 on its 3′ end. For the internal control beta actin specific probe, the signal from the fluorescent dye (Cy5) on the 5′end is quenched by BHQ-1 on its 3′ end.

### Pentaplex

Same primers as QPlex, however the systems and methods herein include added 1 RdRP probe.

### Duplex

In the systems and methods herein, Beta-actin, RdRP, N-1, N-2, and Orflab have nucleic acid sequences corresponding to Seq ID NO: 36, Seq ID NO: 32, Seq ID NO: 34, Seq ID NO: 35, and Seq ID NO: 33, respectively.

### EXAMPLES

The following examples illustrate the isolation and identification of the novel COVID-19 virus. These examples should not be construed as limiting.

### Example 1—N Marker

### SARS-Cov-2 N (N-1) (Target)Primer/Probe

GenBank MN908947.3 SARS-Cov-2:N-2 primer/probe 28881-28971 nt
different from who recommended (28555-28682)

TABLE 13
Sequence of Oligonucleotide Primer for Detecting N-1 of SARS-Cov-2

| | | Nucleotide site | |
|---|---|---|---|
| | Sequence | (GenBank: | |
| Name | number | MN908947.3 | Sequence |
| Forward primer of N-1 | SEQ ID | Nt 28881-28902 | 5′-(GGGGAACTTCT |
| of SARS-Cov-2 | NO: 20 | | CCTGCTAGAAT-3′ |
| Reverse primer of N-1 | SEQ ID | Nt 28952-28971 | 5′-TTGCTCTCA |
| of SARS-Cov-2 | NO: 21 | | AGCTGGTTCAA-3′ |

(1) Primers are made by Oligonucleotide primer production companies such as Cosmo Gentec, or Bionix, with special treatment (OPC, HAP, respectively) to ensure purity.
(2) 8 Primers that have passed the basic testing (outside inspection, check manufacturer's attached validation insert, CoA) should be dissolved in the adequate volume of distilled water to make a concentrated stock solution of 100 pmole/ul (=100 uM).
(3) Dilute the 8 primer working solutions into concentrations of 5 pmol/μl each by mixing 50 uL of 100 uM primer in 720 uL of distilled water. Vortex for 1 minute. Measure concentration with spectrophotometer.
(4) Perform Realtime PCR with Standard material (Performance test) Alternatively, primer quality can be checked on a denaturing polyacrylamide gel; a single band should be seen.
(5) Sequencing using finished product (Efficacy test)
(6) Aliquot decided amount depending on PCR regimen to avoid repeated thawing and freezing.
(7) Label finished product. Store all primer solutions at −20° C.

TABLE 14
Sequence of Oligonucleotide Probe for Detecting N-1 of SARS-Cov-2

| | | Nucleotide | |
|---|---|---|---|
| | | site | |
| | Sequence | (GenBank: | |
| Name | number | MN908947.3 | Sequence |
| Potential site of probe | SEQ ID | Nt 28903-28951 | 5′- GGAACTTC TCCTGCTAGA |
| for N of SARS-Cov-2 | NO: 22 | | ATGGCTGGCA ATGGCGGTGA |
| | | | TGCTGCTCTT GCTTTGCTGC |
| | | | TGCTTGACAG ATTGAACCA -3′ |
| Probe of N used in the | SEQ ID | Nt 28934-28953 | 5′-/TexRed/ TTG CTG CTG CTT |
| The Product” | NO: 11 | | GAC AGA TT/ BHQ_2/-3′ |

All of 15-30 mer sequences within Seq ID NOs listed above to use as probe
All combinations of 15-30 mer sequences within Seq ID NOs are describes below.
(1) Probes are made by Dual label Modification oligonucleotide probe production companies such as IDT, Bioneer, Cosmo Gentec, or BioBasic, with special treatment (HPLC) to ensure purity.
(2) 4 Probes that have passed the basic testing (outside inspection, check manufacturer's attached validation insert, CoA with mass spectrometry data) should be dissolved in the adequate volume of distilled water to make a concentrated stock solution of 100 pmole/ul (=100 uM).
(3) Dilute the 4 probe working solutions into concentrations of 2 pmol/μl each by mixing 4 uL of 100 uM probe in 784 uL of distilled water. Vortex for 1 minute. Measure concentration with spectrophotometer.
(4) Perform Realtime PCR with Standard material (Performance test)
(5) Sequencing using finished product (Efficacy test)
(6) Aliquot 150 ul per vial to avoid repeated thawing and freezing.
(7) Label finished product. Store all probe solutions at −20° C.
Probe: Taqman 5′reporter, 3′quencher.
Reporter included: FAM(6-carboxyfluorescein), (texas red), (fluorescein), HEX(2′,4′,5′,7′-tetrachloro-6-carboxy-4,7-dichlorofluorescein), (fluorescein chlorotriazinyl), (rhodamine green), (rhodamine red), (tetramethylrhodamine), FITC(fluorescein isothiocyanate), (oregon green), (alexa fluor), JOE (6-Carboxy-4′,5′ Dichloro-2′,7′-Dimethoxyfluorescein), ROX (6-Carboxyl-X-Rhodamine), TET (Tetrachloro-Fluorescein), TRITC (tertramethylrodamine isothiocyanate), TAMRA (6-carboxytetramethyl-rhodamine), NED (N-(1-Naphthyl) ethylenediamine, (Cyanine) (thiadicarbocyanine).
Quencher included: TAMRA(6-carboxytetramethyl-rhodamine), BHQ1 (black hole quencher 1), BHQ2 (black hole quencher 2), BHQ3 (black hole quencher 3), NFQ (nonfluorescent quencher), (dabcyl), Eclipse, DDQ(Deep Dark Quencher), (Blackberry Quencher), (Iowa black).

SARS-Cov-2 N (N-2) (Target)Primer/Probe Set
GenBank MN908947.3 SARS-Cov-2:N-2 primer/probe 28863-28980 nt
different from who recommended (28555-28682)

TABLE 15
Sequence of Oligonucleotide Primer for Detecting N-2 of SARS-Cov-2

| | | Nucleotide site | |
|---|---|---|---|
| | Sequence | (GenBank: | |
| Name | number | MN908947.3 | Sequence |
| Forward primer of N-2 | SEQ ID | Nt 28863-28882 | 5′- CAACTCCAGGCAGTAGG - |
| of SARS-Cov-2 | NO: 1 | | 3′ |
| Reverse primer of N-2 | SEQ ID | Nt 28960-28980 | 5′ CCAGACATTT |
| of SARS-Cov-2 | NO: 2 | | TGCTCTCAAGC -3′ |

(1) Primers are made by Oligonucleotide primer production companies such as Cosmo Gentec, or Bionix, with special treatment (OPC, HAP, respectively) to ensure purity.
(2) 8 Primers that have passed the basic testing (outside inspection, check manufacturer's attached validation insert, CoA) should be dissolved in the adequate volume of distilled water to make a concentrated stock solution of 100 pmole/ul (=100 uM).
(3) Dilute the 8 primer working solutions into concentrations of 5 pmol/μl each by mixing 50 uL of 100 uM primer in 720 uL of distilled water. Vortex for 1 minute. Measure concentration with spectrophotometer.
(4) Perform Realtime PCR with Standard material (Performance test) Alternatively, primer quality can be checked on a denaturing polyacrylamide gel; a single band should be seen.
(5) Sequencing using finished product (Efficacy test)
(6) Aliquot decided amount depending on PCR regimen to avoid repeated thawing and freezing.
(7) Label finished product. Store all primer solutions at −20° C.

TABLE 16
Sequence of Oligonucleotide Probe for Detecting N-2 of SARS-Cov-2

| | | Nucleotide site | |
|---|---|---|---|
| | Sequence | (GenBank: | |
| Name | number | MN908947.3 | Sequence |
| Potential site of | SEQ ID | Nt 28885-28951 | 5′- GGAACTTCTC |
| probe for N of | NO: 9 | | CTGCTAGAAT GGCTGGCAAT |
| SARS-Cov-2 | | | GGCGGTGATG CTGCTCTTGC |
| | | | TTTGCTGCTG CTTGACAGAT |
| | | | TGAACCA -3′ |
| Probe of N used in | SEQ ID | Nt 28939-28956 | 5′-/TexRed/ TTG CTG CTG CTT |
| the “The Product” | NO: 11 | | GAC AGA TT/ BHQ_2/-3′ |

All of 15-30 mer sequences within Seq ID NOs above to use as probe.
All combinations of 15-30 mer sequences within Seq ID NOs above and below.
(1) Probes are made by Dual label Modification oligonucleotide probe production companies such as IDT, Bioneer, Cosmo Gentec, or BioBasic, with special treatment (HPLC) to ensure purity.
(2) 4 Probes that have passed the basic testing (outside inspection, check manufacturer's attached validation insert, CoA with mass spectrometry data) should be dissolved in the adequate volume of distilled water to make a concentrated stock solution of 100 pmole/ul (=100 uM).
(3) Dilute the 4 probe working solutions into concentrations of 2 pmol/μl each by mixing 4 uL of 100 uM probe in 784 uL of distilled water. Vortex for 1 minute. Measure concentration with spectrophotometer.
(4) Perform Realtime PCR with Standard material (Performance test)
(5) Sequencing using finished product (Efficacy test)
(6) Aliquot 150 ul per vial to avoid repeated thawing and freezing.
(7) Label finished product. Store all probe solutions at −20° C.
Probe: Taqman 5′reporter, 3′quencher.
Reporter included: FAM(6-carboxyfluorescein), (texas red), (fluorescein), HEX(2′,4′,5′,7′-tetrachloro-6-carboxy-4,7-dichlorofluorescein), (fluorescein chlorotriazinyl), (rhodamine green), (rhodamine red), (tetramethylrhodamine), FITC(fluorescein isothiocyanate), (oregon green), (alexa fluor), JOE (6-Carboxy-4′,5′ Dichloro-2′,7′-Dimethoxyfluorescein), ROX (6-Carboxyl-X-Rhodamine), TET (Tetrachloro-Fluorescein), TRITC (tertramethylrodamine isothiocyanate), TAMRA (6-carboxytetramethyl-rhodamine), NED (N-(1-Naphthyl) ethylenediamine, (Cyanine) (thiadicarbocyanine).
Quencher included: TAMRA(6-carboxytetramethyl-rhodamine), BHQ1 (black hole quencher 1), BHQ2 (black hole quencher 2), BHQ3 (black hole quencher 3), NFQ (nonfluorescent quencher), (dabcyl), Eclipse, DDQ(Deep Dark Quencher), (Blackberry Quencher), (Iowa black).

### Example 2—RdRP Gene Probe/Primer

TABLE 17
Sequence of Oligonucleotide Primer for Detecting RdRp of SARS-Cov-2

| | | Nucleotide site | |
|---|---|---|---|
| | Sequence | (GenBank: | |
| Name | number | MN908947.3 | Sequence |
| Forward primer of RdRP | SEQ ID | Nt 15441-15460 | 5′- CATGTGTGGCGGTTCACTAT- -3′ |
| of SARS-Cov-2 | NO: 5 | | |
| Reverse primer of RdRP | SEQ ID | Nt 15500-15526 | 5′TGTTAAAAACACT |
| of SARS-Cov-2 | NO: 6 | | ATTAGCATAAGCAG -3′ |

location 15441~15526 bp
who: (15361-15460)

(1) Primers are made by Oligonucleotide primer production companies such as Cosmo Gentec, or Bionix, with special treatment (OPC, HAP, respectively) to ensure purity.
(2) 8 Primers that have passed the basic testing (outside inspection, check manufacturer's attached validation insert, CoA) should be dissolved in the adequate volume of distilled water to make a concentrated stock solution of 100 pmole/ul (=100 uM).
(3) Dilute the 8 primer working solutions into concentrations of 5 pmol/μl each by mixing 50 uL of 100 uM primer in 720 uL of distilled water. Vortex for 1 minute. Measure concentration with spectrophotometer.
(4) Perform Realtime PCR with Standard material (Performance test). Alternatively, primer quality can be checked on a denaturing polyacrylamide gel; a single band should be seen.
(5) Sequencing using finished product (Efficacy test)
(6) Aliquot decided amount depending on PCR regimen to avoid repeated thawing and freezing.
(7) Label finished product. Store all primer solutions at −20° C.

TABLE 18
Sequence of Oligonucleotide Probe for Detecting RdRp of SARS-Cov-2

| | | Nucleotide site | |
|---|---|---|---|
| | Sequence | (GenBank: | |
| Name | number | MN908947.3 | Sequence |
| Potential site of probe for | SEQ ID | Nt 15461-15499 | 5′- ATG TTA AAA CCA GGT GGA ACC |
| RdRP of SARS-Cov-2 | NO: 15 | | TCA GGA GAT GCC ACA AAC -3′ |
| Probe of RdRP used in the | SEQ ID | Nt 15470-15494 | 5′-[FAM]/ |
| “The Product” | NO: 17 | | CAGGTGGAACCTCATCAGGAGATGC/ |
| | | | [BHQ1]- 3′ |

All of 15-30 mer sequences within Seq ID NOs above to use as probe.
All combinations of 15-30 mer sequences within Seq ID NOs above and below.
(1) Probes are made by Dual label Modification oligonucleotide probe production companies such as IDT, Bioneer, Cosmo Gentec, or BioBasic, with special treatment (HPLC) to ensure purity.
(2) 4 Probes that have passed the basic testing (outside inspection, check manufacturer's attached validation insert, CoA with mass spectrometry data) should be dissolved in the adequate volume of distilled water to make a concentrated stock solution of 100 pmole/ul (=100 uM).
(3) Dilute the 4 probe working solutions into concentrations of 2 pmol/μl each by mixing 4 uL of 100 uM probe in 784 uL of distilled water. Vortex for 1 minute. Measure concentration with spectrophotometer.
(4) Perform Realtime PCR with Standard material (Performance test)
(5) Sequencing using finished product (Efficacy test)
(6) Aliquot 150 ul per vial to avoid repeated thawing and freezing.
(7) Label finished product. Store all probe solutions at −20° C.
Probe: Taqman 5′reporter, 3′quencher.
Reporter included: FAM(6-carboxyfluorescein), (texas red), (fluorescein), HEX(2′,4′,5′,7-tetrachloro-6-carboxy-4,7-dichlorofluorescein), (fluorescein chlorotriazinyl), (rhodamine green), (rhodamine red), (tetramethylrhodamine), FITC(fluorescein isothiocyanate), (oregon green), (alexa fluor) JOE (6-Carboxy-4′,5′ Dichloro-2′,7-Dimethoxyfluorescein), ROX (6-Carboxyl-X-Rhodamine), TET (Tetrachloro-Fluorescein), TRITC(tetramethylrhodamine isocyanate), TAMRA (6-carboxytetramethyl-rhodamine), NED (N-(1-Naphthyl) ethylenediamine, (Cyanine) (thiadicarbocyanine).
Quencher included: TAMRA(6-carboxytetramethyl-rhodamine), BHQ1 (black hole quencher 1), BHQ2 (black hole quencher 2), BHQ3 (black hole quencher 3), NFQ (nonfluorescent quencher), (dabcyl), Eclipse, DDQ(Deep Dark Quencher), (Blackberry Quencher), (Iowa black).

### Example 3—SARS-Cov-2 ORFlab (Target) Primer/Probe

TABLE 19
Sequence of Oligonucleotide Primer for Detecting ORF1ab of SARS-Cov- 2

| | | Nucleotide site | |
|---|---|---|---|
| | Sequence | (GenBank: | |
| Name | number | MN908947.3 | Sequence |
| Forward primer of Orf1ab of | SEQ ID NO: | Nt 13348-13372 | 5′- GGGTTTTACACTT |
| SARS-Cov-2 | 3 | | AAAAACACAGTC -3′ |
| Reverse primer of Orf1ab of | SEQ ID NO: | Nt 13433-13452 | 5′GCATCAGCTGACTGAAGCAT -3′ |
| SARS-Cov-2 | 4 | | |

GenBank MN908947.3 SARS-Cov-2: Orf1ab primer/probe 13348-13452 nt different from who recommended (15361-15460)

(1) Primers are made by Oligonucleotide primer production companies such as Cosmo Gentec, or Bionix, with special treatment (OPC, HAP, respectively) to ensure purity.
(2) 8 Primers that have passed the basic testing (outside inspection, check manufacturer's attached validation insert, CoA) should be dissolved in the adequate volume of distilled water to make a concentrated stock solution of 100 pmole/ul (=100 uM).
(3) Dilute the 8 primer working solutions into concentrations of 5 pmol/μl each by mixing 50 uL of 100 uM primer in 720 uL of distilled water. Vortex for 1 minute. Measure concentration with spectrophotometer.
(4) Perform Realtime PCR with Standard material (Performance test) Alternatively, primer quality can be checked on a denaturing polyacrylamide gel; a single band should be seen.
(5) Sequencing using finished product (Efficacy test)
(6) Aliquot decided amount depending on PCR regimen to avoid repeated thawing and freezing.
(7) Label finished product. Store all primer solutions at −20° C.

TABLE 20
Sequence of Oligonucleotide Probe for Detecting ORF1ab of SARS-Cov-2

| | | Nucleotide site | |
|---|---|---|---|
| | Sequence | (GenBank: | |
| Name | number | MN908947.3 | Sequence |
| Potential site of probe for | SEQ ID NO: 12 | Nt 13373-13432 | 5′- TGT ACC GTC TGC GGT ATG |
| Orf1ab of SARS-Cov-2 | | | TGG AAA GGT TAT GGC TGT |
| | | | AGT TGT GAT CAA CTC CGC |
| | | | GAA CCC -3′ |
| Probe of Orf1ab used in | SEQ ID NO: 14 | Nt 13377-13404 | 5′-[Cyanine5]/CCG TCT GCG GTA |
| the “The Product” | | | TGT GGA AAG GTT ATG |
| | | | G/[BHQ2]- 3′ |

All of 15-30 mer sequences within Seq ID NOs disclosed above to use as probe
All combinations of 15-30 mer sequences within Seq ID NOs disclosed above and below.
(1) Probes are made by Dual label Modification oligonucleotide probe production companies such as IDT, Bioneer, Cosmo Gentec, or BioBasic, with special treatment (HPLC) to ensure purity.
(2) 4 Probes that have passed the basic testing (outside inspection, check manufacturer's attached validation insert, CoA with mass spectrometry data) should be dissolved in the adequate volume of distilled water to make a concentrated stock solution of 100 pmole/ul (=100 uM).
(3) Dilute the 4 probe working solutions into concentrations of 2 pmol/μl each by mixing 4 uL of 100 uM probe in 784 uL of distilled water. Vortex for 1 minute. Measure concentration with spectrophotometer.
(4) Perform Realtime PCR with Standard material (Performance test).
(5) Sequencing using finished product (Efficacy test).
(6) Aliquot 150 ul per vial to avoid repeated thawing and freezing.
(7) Label finished product. Store all probe solutions at −20° C.
Probe: Taqman 5′reporter, 3′quencher.
Reporter included: FAM(6-carboxyfluorescein), (texas red), (fluorescein), HEX(2′,4′,5′,7′-tetrachloro-6-carboxy-4,7-dichlorofluorescein), (fluorescein chlorotriazinyl), (rhodamine green), (rhodamine red), (tetramethylrhodamine), FITC(fluorescein isothiocyanate), (oregon green), (alexa fluor), JOE (6-Carboxy-4′,5′ Dichloro-2′,7′-Dimethoxyfluorescein), ROX (6-Carboxyl-X-Rhodamine), TET (Tetrachloro-Fluorescein), TRITC (tertramethylrodamine isothiocyanate), TAMRA (6-carboxytetramethyl-rhodamine), NED (N-(1-Naphthyl) ethylenediamine, (Cyanine) (thiadicarbocyanine).
Quencher included: TAMRA(6-carboxytetramethyl-rhodamine), BHQ1 (black hole quencher 1), BHQ2 (black hole quencher 2), BHQ3 (black hole quencher 3), NFQ (nonfluorescent quencher), (dabcyl), Eclipse, DDQ(Deep Dark Quencher), (Blackberry Quencher), (Iowa black).

### Example 4—Internal Controls

1. Internal control-1 primer/probe: beta-actin (target)
342˜443 nt location. (NM_001101.5)

TABLE 21
Sequence of Oligonucleotide Primer and Probe for Detecting beta
actin of human internal control (NM_001101.5)

| | | Nucleotide site | |
|---|---|---|---|
| | Sequence | (GenBank: | |
| Name | number | NM_001101.5) | Sequence |
| Forward primer | SEQ ID | Nt 242-262 | 5′-CATGTGTGGCGGTTCACTAT-3′ |
| of beta-actin | NO: 15 | | |
| Reverse primer | SEQ ID | Nt 424-443 | 5′TGTTAAAAACAC |
| of beta-actin | NO: 16 | | TATTAGCATAACGAG-3′ |
| Probe of beta- | SEQ ID | Nt 385-407 | 5′-CAGGTGGAACC |
| actin | NO: 18 | | TCATCAGGAGATGC-3′ |

1. Primer production:
(1) Primers are made by Oligonucleotide primer production companies such as Cosmo Gentec, or Bionix, with special treatment (OPC, HAP, respectively) to ensure purity.
(2) 8 Primers that have passed the basic testing (outside inspection, check manufacturer's attached validation insert, CoA) should be dissolved in the adequate volume of distilled water to make a concentrated stock solution of 100 pmole/ul (=100 uM).
(3) Dilute the 8 primer working solutions into concentrations of 5 pmol/μl each by mixing 50 uL of 100 uM primer in 720 uL of distilled water. Vortex for 1 minute. Measure concentration with spectrophotometer.
(4) Perform Realtime PCR with Standard material (Performance test) Alternatively, primer quality can be checked on a denaturing polyacrylamide gel; a single band should be seen.
(5) Sequencing using finished product (Efficacy test)
(6) Aliquot decided amount depending on PCR regimen to avoid repeated thawing and freezing.
(7) Label finished product. Store all primer solutions at −20° C.
2. Probe production:
(1) Probes are made by Dual label Modification oligonucleotide probe production companies such as IDT, Bioneer, Cosmo Gentec, or BioBasic, with special treatment (HPLC) to ensure purity.
(2) 4 Probes that have passed the basic testing (outside inspection, check manufacturer's attached validation insert, CoA with mass spectrometry data) should be dissolved in the adequate volume of distilled water to make a concentrated stock solution of 100 pmole/ul (=100 uM).
(3) Dilute the 4 probe working solutions into concentrations of 2 pmol/μl each by mixing 4 uL of 100 uM probe in 784 uL of distilled water. Vortex for 1 minute. Measure concentration with spectrophotometer.
(4) Perform Realtime PCR with Standard material (Performance test).
(5) Sequencing using finished product (Efficacy test).
(6) Aliquot 150 ul per vial to avoid repeated thawing and freezing.
(7) Label finished product. Store all probe solutions at −20° C.
Probe: Taqman 5′reporter, 3′quencher.
Reporter included: FAM(6-carboxyfluorescein), (texas red), (fluorescein), HEX(2′,4′,5′,7′-tetrachloro-6-carboxy-4,7-dichlorofluorescein), (fluorescein chlorotriazinyl), (rhodamine green), (rhodamine red), (tetramethylrhodamine), FITC(fluorescein isothiocyanate), (oregon green), (alexa fluor), JOE (6-Carboxy-4′,5′ Dichloro-2′,7′-Dimethoxyfluorescein), ROX (6-Carboxyl-X-Rhodamine), TET (Tetrachloro-Fluorescein), TRITC (tertramethylrodamine isothiocyanate), TAMRA (6-carboxytetramethyl-rhodamine), NED (N-(1-Naphthyl) ethylenediamine, (Cyanine) (thiadicarbocyanine).
Quencher included: TAMRA(6-carboxytetramethyl-rhodamine), BHQ1 (black hole quencher 1), BHQ2 (black hole quencher 2), BHQ3 (black hole quencher 3), NFQ (nonfluorescent quencher), (dabcyl), Eclipse, DDQ(Deep Dark Quencher), (Blackberry Quencher), (Iowa black).

### #Validation:

Standard material marker concentration:
3×-10× LoD; viral genomic RNA copy 100 cp/ul.
internal control material IC marker 100 cp/ul (Table 22)

TABLE 22
Verification of Internal Control

| | |
|---|---|
| Concentration of IC | IC |

| (cp/ul) | Detection rate | Ct (mean +/− SD) |
|---|---|---|
| 1 × 10\hat{ }2 | 20/20 | 31.31 +/− 0.48 |
| 3 × 10\hat{ }1 | 20/20 | 35.01 +/− 0.38 |
| 1 × 10\hat{ }1 | 20/20 | 38.04 +/− 0.57 |
| 3 | 0/20 | N.D |
| 1 | 0/20 | N.D |

Abbreviation) N.D.: Not detectable.
The cut off level of IC is set at ≤40

2. Internal control-2 primer/probe: Pbgd (target)
Location:21-147 nt (NM_000190.4)

TABLE 23
Sequence of Oligonucleotide Primer and Probe for Detecting
Pbgd of human internal control (NM_000190.4)

| | | Nucleotide | |
|---|---|---|---|
| | | site | |
| | Sequence | (GenBank: | |
| Name | number | NM_000190.4) | Sequence |
| Forward primer of | SEQ ID | Nt 21-40 | 5′-CATGTGTGGC |
| PBGD | NO: 13 | | GGTTCACTAT-3′ |
| Reverse primer of PBGD | SEQ ID | Nt 128-147 | 5′TGTTAAAAACACT |
| | NO: 14 | | ATTAGCATAACGAG-3′ |
| Probe of PBGD | SEQ ID | Nt 58-75 | 5′-CAGGTGGAACCT |
| | NO: 17 | | CATCAGGAGATGC-3′ |

1. Primer production:
(1) Primers are made by Oligonucleotide primer production companies such as Cosmo Gentec, or Bionix, with special treatment (OPC, HAP, respectively) to ensure purity.
(2) 8 Primers that have passed the basic testing (outside inspection, check manufacturer's attached validation insert, CoA) should be dissolved in the adequate volume of distilled water to make a concentrated stock solution of 100 pmole/ul (=100 uM).
(3) Dilute the 8 primer working solutions into concentrations of 5 pmol/μl each by mixing 50 uL of 100 uM primer in 720 uL of distilled water. Vortex for 1 minute. Measure concentration with spectrophotometer.
(4) Perform Realtime PCR with Standard material (Performance test). Alternatively, primer quality can be checked on a denaturing polyacrylamide gel; a single band should be seen.
(5) Sequencing using finished product (Efficacy test).
(6) Aliquot decided amount depending on PCR regimen to avoid repeated thawing and freezing.
(7) Label finished product. Store all primer solutions at −20° C.
2. Probe production:
(1) Probes are made by Dual label Modification oligonucleotide probe production companies such as IDT, Bioneer, Cosmo Gentec, or BioBasic, with special treatment (HPLC) to ensure purity.
(2) 4 Probes that have passed the basic testing (outside inspection, check manufacturer's attached validation insert, CoA with mass spectrometry data) should be dissolved in the adequate volume of distilled water to make a concentrated stock solution of 100 pmole/ul (=100 uM).
(3) Dilute the 4 probe working solutions into concentrations of 2 pmol/μl each by mixing 4 uL of 100 uM probe in 784 uL of distilled water. Vortex for 1 minute. Measure concentration with spectrophotometer.
(4) Perform Realtime PCR with Standard material (Performance test).
(5) Sequencing using finished product (Efficacy test).
(6) Aliquot 150 ul per vial to avoid repeated thawing and freezing.
(7) Label finished product. Store all probe solutions at −20° C.

### Example 5—Internal Control and Negative Control

1) COVID-19 Internal Control-1:
   1) This contains in vitro transcript RNA of internal control gene (human beta-actin) (100 cp/ul) 2) Use through the entire sample processing procedure, excluding the extraction.
   3) Used to prove the functionality of the reaction mix for amplification of the pathogen target and rules out inhibition when used together with IC
   4) Precautions: This reagent should be handled with caution in a dedicated nucleic acid handling area to prevent possible contamination Maintain on ice when thawed.
   5) Aliquot 1 ul upon receival and store at ≤−20° C. until use. Avoid repeated freeze-thaw cycles.

TABLE 24
Sequence of Internal Control Material [Claim 5-1]

| | Length | |
|---|---|---|
| RNA | (mer) | Base sequence |
| beta | 102 | 5′-GGCACCACACCTTCTACAATGAGCTGCGTGTGGCTCCCGAGGAG |
| actin | | CACCCCGTGCTGCTGACCGAGGCCCCCCTGAACCCCAAGGCCAACC |
| | | GCGAGAAGATGA-3′ |

### Production:

(1) Internal Control that is cloned to plasmid (pGEM T-Easy Vector (promega) is used; amplified by E. coli culture) into the plasmid.
(2) Extract the plasmid RNA with TransciptAid T7 high Yield Transcription kit.
(3) Measure concentration with spectrophotometer; dilute the working solution into concentrations of 1.575 ng/ul (1×10\hat{ }10 copy/ul)
(4) Dilute this again by mixing 1 uL of this internal control into 990 uL of distilled water into 10 ul of working solution at (3). Vortex for 1 minute.
(5) Perform Realtime PCR with Standard material and package 20 ul per vial.
(6) Label finished product. Store all control solutions at −20° C.
(2) COVID-19 Internal Control-2:
   1) This contains in vitro transcript RNA of internal control gene (Pbgd) (100 cp/ul)
   2) Use through the entire sample processing procedure, excluding the extraction.
   3) Used to prove the functionality of the reaction mix for amplification of the pathogen target and rules out inhibition when used together with IC
   4) Precautions: This reagent should be handled with caution in a dedicated nucleic acid handling area to prevent possible contamination Maintain on ice when thawed.
   5) Aliquot 1 ul upon receival and store at ≤−20° C. until use. Avoid repeated freeze-thaw cycles.

TABLE 25
Sequence of Internal Control Material [Claims 5-2]

| | Length | |
|---|---|---|
| RNA | (mer) | Base sequence |
| Pbgd | 127 | 5′- GAGACCAGGA GTCAGACTGT AGGACGACCT CGGGTCCCAC |
| | | GTGTCCCCGG TACTCGCCGG CCGGAGCCCC CGGCTTCCCG |
| | | GGGCCGGGGG ACCTTAGCGG CACCCACACA CAGCCTACTT |
| | | TCCAAGC -3′ |

### Production:

(1) Internal Control that is cloned to plasmid (pGEM T-Easy Vector (promega) is used; amplified by E. coli culture) into the plasmid.
(2) Extract the plasmid RNA with TransciptAid T7 high Yield Transcription kit.
(3) Measure concentration with spectrophotometer; dilute the working solution into concentrations of 1.375 ng/ul (1×10\hat{ }10 copy/ul)
(4) Dilute this again by mixing 1 uL of this internal control into 999 uL of distilled water. Vortex for 1 minute.
(5) Perform Realtime PCR with Standard material and package 20 ul per vial.
(6) Label finished product. Store all control solutions at −20° C.
#Note: 2 other negative controls are claimed
(1) No Template Control (Negative Control; NTC; NC) (not Provided with Kit)
1) Sterile, nuclease-free water of same quantity as the template on every run.
2) Used to check for contamination during PCR plate set-up.
3) Use through the entire sample processing procedure, including the extraction.
(2) Negative Extraction Control (NEC) (not Provided with Kit)
1) Clinical patient specimen that has been previously tested negative, prepared by extracting RNA of same quantity as the template on every run.
2) Used as the negative extraction control for the entire testing system to check adequacy of RNA extraction and to check for contamination during PCR plate set-up.
3) Prepare at least 1 negative extraction control (NEC) each time RNA is extracted from a clinical specimen or sample. NEC is added to extraction system.

### Example 6—Positive Control

1. COVID-19 Positive Control-1

1) SARS-Cov-2 virus genomic RNA (Total genomic RNA of SARS-Cov-2; MT007544.1 and MN908947.3 Genbank; Twist Bioscience, San Francisco, Calif., 10\hat{ }6 copies/ul) is diluted to 100 cp/ul for use in all kits except Triplex-1 (US CDC approved standard material).
* Due to mix with RNA carrier, low levels of human housekeeping gene may be present and may cause low levels (Ct 38-40) of beta actin or Pbgd positivity, but does not cause problems in interpretation (Previous experiment proven).
2) Use through the entire sample processing procedure, excluding the extraction.
3) Use to prove the functionality of the reaction mix for amplification of the pathogen target and rules out inhibition when used together with IC.
4) Precautions: This reagent should be handled with caution in a dedicated nucleic acid handling area to prevent possible contamination Maintain on ice when thawed.
5) Aliquot 3 ul upon receival and store at ≤−20° C. until use. Avoid repeated freeze-thaw cycles.

2. COVID-19 Positive Control-2:

1) This is a mixture of in vitro transcript RNA of N, ORFlab of SARS-Cov-2 (100 cp/ul) for use in Triplex-1 only.
2) Use through the entire sample processing procedure, excluding the extraction.
3) Used to prove the functionality of the reaction mix for amplification of the pathogen target and rules out inhibition when used together with IC.
4) Precautions: This reagent should be handled with caution in a dedicated nucleic acid handling area to prevent possible contamination Maintain on ice when thawed.
5) Aliquot 3 ul upon receival and store at ≤−20° C. until use. Avoid repeated freeze-thaw cycles.

TABLE 26
Sequence of Positive Control-2 Material

| RNA | length (mer) | Base sequence |
|---|---|---|
| N | 99 | 5′- |
| | | GGGGAACTTCTCCTGCTAGAATGGCTGGCAATGGCGGTGATGCTGCTCT |
| | | TGCTTTGCTGCTGCTTGACAGATTGAACCAGCTTGAGAGCAAAATGTCT |
| | | G-3′ |
| ORF1ab | 119 | 5′- |
| | | CCCTGTGGGTTTTACACTTAAAAACACAGTCTGTACCGTCTGCGGTATG |
| | | TGGAAAGGTTATGGCTGTAGTTGTGATCAACTCCGCGAACCCATGCTTC |
| | | AGTCAGCTGATGCACAATCGT-3′ |

### 6) Production:

(1) Positive Control cloned to plasmid (pGEM T-Easy Vector (promega) is used; amplified by E. coli culture) into the plasmid.
(2) Extract the plasmid RNA with TransciptAid T7 high Yield Transcription kit.
(3) Measure concentration with spectrophotometer; dilute the working solution into concentrations of 1.07 ng/ul for N positive control (1×10\hat{ }10 copy/ul) and 1.29 ng/uL for orflab positive control (1×10\hat{ }10 copy/ul).
(4) Dilute again by mixing 1 uL of ORF-lab and 1 uL of N and 1 uL of RdRP positive control into 998 uL of distilled water. Vortex for 1 minute.
(5) Perform Realtime PCR with Standard material and package 60 ul per vial.
(6) Label finished product. Store all control solutions at −20° C.

### Example 7—Realtime RT PCR and Kit Constituents (Preparation of Real Time RT-PCR Reagent)

All PCR Except Triplex-1
   * Takara One Step RT-PCR kit (One Step Prime Script RT-PCR kit, Cat No: RR064A, RR064B, Takara Bio, Nojihigashi 7-4-38, Kusatsu, Shiga, 525-0058, Japan, Japan)

TABLE 27
constituent of All PCR (except Triplex-1)
Reagents used (100 reactions)

| Reagent Number | Reagent Label | Constituent | Description | Unit Amount | | Specification** | Unit | |
|---|---|---|---|---|---|---|---|---|
| 1 | Primer Mix | Main | N-2 Forward primer | 5 | pmol/ul | Provided | 300 | ul/vial |
| | | | oligonucleotides | | | | | |
| | | Main | N-2 Reverse primer | 5 | pmol/ul | Provided | | |
| | | | oligonucleotides | | | | | |
| | | Main | RdRp Forward primer | 5 | pmol/ul | Not provided | | |
| | | | oligonucleotides | | | | | |
| | | Main | RdRp_Reverse primer | 5 | pmol/ul | Not provided | | |
| | | | oligonucleotides | | | | | |
| | | Main | BETA ACTIN-Forward primer | 5 | pmol/ul | Not provided | | |
| | | | oligonucleotides | | | | | |
| | | Main | BETA ACTIN-Reverse primer | 5 | pmol/ul | Not provided | | |
| | | | oligonucleotides | | | | | |
| 2 | Probe Mix | Main | N_5′TexasRed/3′-BHQ2 | 2 | pmol/ul | Not provided | 300 ul/vial, | |
| | | | dual labeled probe | | | | 1 bottle | |
| | | Main | RdRp_FAM/3′-BHQ1 | 2 | pmol/ul | Provided | | |
| | | | dual labeled probe | | | | | |
| | | Main | BETA ACTIN_5′HEX/3′-BHQ1 | 2 | pmol/ul | Provided | | |
| | | | dual labeled probe | | | | | |
| 3 | Hotstart Taq | Main | Hotstart Taq | 5 | U/ul | Not provided | 50 | ul/ |

| | | | | |
|---|---|---|---|---|
| Enzyme | Accessory | Glycerol | Drop dose | Not provided |
| | Accessory | Tris-Cl | Drop dose | Not provided |
| | Accessory | KCl | Drop dose | Not provided |
| | Accessory | Dithiothreitol | Drop dose | Not provided |
| | Accessory | EDTA | Drop dose | Not provided |
| | Accessory | Nonidet P-40 | Drop dose | Not provided |
| | Accessory | Tween ®-20 | Drop dose | Not provided |

| 4 | RNase free water | Main | Distilled water | Drop dose | | Not provided | 600 ul/vial, |
|---|---|---|---|---|---|---|---|
| | | | | | | Not provided | 1 bottle |
| 5 | Internal Control | Main | IC RNA | 1 × 10\hat{ }2 | copy/ul | Not provided | 20 ul/vial, |
| | (IC) | | | | | Not provided | 1 bottle |
| 6 | Positive Control | Main | Orf1ab RNA | 3 × 10\hat{ }2 | copy/ul | Not provided | 60 ul/vial, |
| | (PTC) | | N RNA | | | Not provided | 1 bottle |

| | |
|---|---|
| RdRp RNA | Not provided |

| | | | | | | |
|---|---|---|---|---|---|---|
| 7 | Onestep RT-Enzyme | Main | Reverse transciptase | Drop dose | Not provided | 50 ul/vial, |
| | mix * | Accessory | Tris-Cl | Drop dose | Not provided | 1 bottle |

| | | | |
|---|---|---|---|
| Accessory | KCl | Drop dose | Not provided |
| Accessory | Dithiothreitol | Drop dose | Not provided |
| Accessory | EDTA | Drop dose | Not provided |
| Accessory | Nonidet P-40 | Drop dose | Not provided |
| Accessory | Tween ®-20 | Drop dose | Not provided |
| Accessory | Glycerol | Drop dose | Not provided |

| | | | | | | |
|---|---|---|---|---|---|---|
| 8 | 2xOnestep | Accessory | Tris-Cl | Drop dose | Not provided | 1000 ul/vial, |
| | RT-PCR buffer * | Accessory | KCl | Drop dose | Not provided | 1 bottle |

| Accessory | (NH4)₂SO₄ | Drop dose | | Not provided |
|---|---|---|---|---|
| Accessory | MgCl₂ | Drop dose | | Not provided |
| Accessory | Dithiothreitol | Drop dose | | Not provided |
| Accessory | dATP | 10 | mM | Not provided |
| Accessory | dCTP | 10 | mM | Not provided |
| Accessory | dGTP | 10 | mM | Not provided |
| Accessory | dTTP | 10 | mM | Not provided |

### 1. OneStep RT-PCR Enzyme mix

(1) Basic testing (outside inspection, check manufacturer's attached validation insert)
(2) Perform Realtime PCR with Standard material (Performance test)
(3) Sequencing using finished product (Efficacy test)
(4) Aliquot assigned dose per vial to avoid repeated thawing and freezing.
(5) Label finished product.

### 2.2× TAKARA OneStep RT-PCR Buffer

2 OneStep RT-PCR Buffer with 2 times the concentration of Tris-Cl, KCl, (NH₄)₂SO₄, MgCl₂, DTT according to the manufacturer (Takara).
(1) Basic testing (outside inspection, check manufacturer's attached validation insert)
(2) Perform Realtime PCR with Standard material (Performance test)
(3) Sequencing using finished product (Efficacy test)
(4) Aliquot assigned dose per vial to avoid repeated thawing and freezing.
(5) Label finished product.

### 3. HotStarTaq® DNA Polymerase

Package HotStarTaq® DNA Polymerase according to the manufacturer (Takara)
(1) Basic testing (outside inspection, check manufacturer's attached validation insert)
(2) Perform Realtime PCR with Standard material (Performance test)
(3) Sequencing using finished product (Efficacy test)
(4) Aliquot assigned dose per vial to avoid repeated thawing and freezing.
(5) Label finished product.
4. RNase free water
Package RNase free water according to the manufacturer (Takara).
(1) Basic testing (outside inspection, check manufacturer's attached validation insert)
(2) Perform Realtime PCR with Standard material (Performance test)
(3) Sequencing using finished product (Efficacy test)
(4) Aliquot assigned dose per vial to avoid repeated thawing and freezing.
(5) Label finished product.
Test 1: Raw material check: packaging inspection, check manufacturer's attached validation insert
Test 2: Semi-finished device check: Application test: Perform Realtime PCR with Standard material Performance/Efficiency testing
Test 3: Verification of Standard materials used: Sequencing
Test 4: Inspect finished product: Designee must check all acceptance records and test results and see that records are present and complete.

Components of COVID-19 Quadplex RT Real Time PCR Kit (100 tests)

| | Component | Volume | Voulme/test |
|---|---|---|---|
| SET | (store at −20° C.) | (μl) | (μl) |
| RT-PCR reagents | RT Enzyme Mix | 50 | 0.5 |
| | RT-PCR Buffer (2x) | 1250 | 12.5 |
| | Hotstart taq Enzyme | 50 | 0.5 |
| Primer/Probe/ | Primer Mix (ORF1ab, N, | 400 | 4 |
| Control Set | RdRp, IC) | | |
| | Probe Mix (ORF1ab, N, | 400 | 4 |
| | RdRp, IC) | | |
| | Positive Control (ORF1ab, | 60 | 3 |
| | N, RdRp) | | |
| | Internal Control (IC) | 20 | 1 |
| | RNase-free Water | 500 | Up to 25 |

Components of COVID-19 Triplex-2 RT Real Time PCR Kit (100 tests)

| | Component | Volume | Voulme/test |
|---|---|---|---|
| SET | (store at −20° C.) | (μl)/100 T | (μl) |
| RT-PCR reagents | RT Enzyme Mix | 50 | 0.5 |
| | RT-PCR Buffer (2x) | 1000 | 10 |
| | Taq polymerase | 50 | 0.5 |
| Primer/Probe/ | Primer Mix (RdRP, N, IC) | 300 | 3 |
| Control Set | Probe Mix (RdRP, N, IC) | 300 | 3 |
| | Positive Control (RdRP, N) | 30 | 3 |
| | Internal Control (IC) | 10 | 1 |
| | RNase-free Water | 300 | Up to 20 |

Components of COVID-19 Duplex-1/2/3/4 RT Real Time PCR Kit (100 tests)

| | Component | Volume | Voulme/test |
|---|---|---|---|
| SET | (store at −20° C.) | (μl)/100 T | (μl) |
| RT-PCR reagents | RT Enzyme Mix | 50 | 0.5 |
| | RT-PCR Buffer (2x) | 1000 | 10 |
| | Taq polymerase | 50 | 0.5 |
| Primer/Probe/ | Primer Mix | 200 | 2 |
| Control Set | Probe Mix | 200 | 2 |
| | Positive Control | 20 | 2 |
| | Internal Control (IC) | 10 | 1 |
| | RNase-free Water | 300 | Up to 20 |

### Example 8A—Real Time RT-PCR Reagent Production

Real time RT-PCR reagent for Triplex-1
Qiagen One-step PCR Kit is used for this composition

TABLE 28.1
Constituent of Qiagen One-step PCR Kit

| QIAGEN OneStep RT-PCR Kit | −25 | | −100 | | −1000 | |
|---|---|---|---|---|---|---|
| Catalog no. | 210210 | | 210212 | | 210215 | |
| Number of reactions | −25 | | −100 | | −1000 | |
| QIAGEN OneStep RT-PCR Enzyme Mix (contains the QIAGEN | 50 | μl | 200 | μl | 2 × 1.0 | ml |
| products Omniscript Reverse Transcriptase, Sensiscript Reverse | | | | | | |
| Transcriptase, and HotStarTaq ® DNA Polymerase) | | | | | | |
| QIAGEN OneStep RT-PCR Buffer,* 5x | 350 | μl | 1.15 | ml | 11.5 | ml |
| Q-Solution ®, 5x | 2 | ml | 2.0 | ml | 10.0 | ml |
| dNTP Mix, 10 mM each | 50 | μl | 200 | μl | 2 × 10.0 | ml |
| RNase-free water | 1.9 | ml | 2 × 1.9 | ml | 2 × 20.0 | ml |

| | | | |
|---|---|---|---|
| Quick-Start Protocol | 1 | 1 | 1 |

TABLE 28.2
Constituent of Triplex-1

| Reagent Number | Reagent Label | Constituent | Description | Unit Amount | | Specification** | Unit | |
|---|---|---|---|---|---|---|---|---|
| 1 | Primer Mix | Main | Primer | 5 | pmol/ul | Provided | 150 | ul/vial |
| 2 | Probe Mix | Main | Probe | 2 | pmol/ul | Provided | 150 | ul/vial |
| 3 | dNTP | Main | dATP | 10 | mM | Not provided | 50 | ul/vial |
| | | Main | dCTP | 10 | mM | Not provided | | |
| | | Main | dGTP | 10 | mM | Not provided | | |
| | | Main | dTTP | 10 | mM | Not provided | | |
| 4 | RNase-free water | Main | Distilled water | Drop dose | | Not provided | 300 | ul/vial |
| 5 | Internal Control | Main | Plasmid | 1 × 10\hat{ }2 | copy/ul | Provided | 30 | ul/vial |
| 6 | Positive Control | Main | Plasmid | 1 × 10\hat{ }2 | copy/ul | Provided | | |

| | | | | | | | |
|---|---|---|---|---|---|---|---|
| 7 | QIAGEN OneStep | Main | HotStar Taq DNA polymerase | Drop dose | Not provided | 50 | ul/vial |
| | RT-PCR Enzyme mix | Main | Reverse Transciptase | Drop dose | Not provided | | |
| | (Quiagen, Hilden, | Accessory | Tris-Cl | Drop dose | Not provided | | |
| | Germany) | Accessory | KCl | Drop dose | Not provided | | |
| | | Accessory | Dithiothreitol | Drop dose | Not provided | | |
| | | Accessory | EDTA | Drop dose | Not provided | | |
| | | Accessory | Nonidet P-40 | Drop dose | Not provided | | |
| | | Accessory | Tween ®-20 | Drop dose | Not provided | | |
| | | Accessory | Glycerol | Drop dose | Not provided | | |
| 8 | 5xOnestep | Accessory | Tris-Cl | Drop dose | Not provided | 200 | ul/vial |
| | RT-PCR buffer | Accessory | KCl | Drop dose | Not provided | | |
| | | Accessory | (NH4)2SO4 | Drop dose | Not provided | | |
| | | Accessory | MgCl2 | Drop dose | Not provided | | |
| | | Accessory | Dithiothreitol | Drop dose | Not provided | | |

1. QIAGEN One Step RT-PCR Enzyme mix (QIAGEN OneStcp RT-PCR Kit Catalog No. 210212/100 reaction)
Use Onestep RT-PCR Enzyme mix contains the QIAGEN products Omniscript Reverse Transcriptase, Sensiscript Reverse Transcriptase, and HotStarTaq® DNA Polymerase as according to the manufacturer

### (Qiagen)

(1) Basic testing (outside inspection, check manufacturer's attached validation insert)
(2) Perform Realtime PCR with Standard material (Performance test)
(3) Sequencing of finished product (Efficacy test)
(4) Aliquot 50 ul per vial to avoid repeated thawing and freezing.
(5) Label finished product.

### 2. 5× QIAGEN OneStep RT-PCR Buffer

5× QIAGEN OneStep RT-PCR Buffer with 5 times the concentration of Tris-Cl, KCl, (NH₄)₂SO₄, MgCl₂, DTT according to the manufacturer (Quiagen).
(1) Basic testing (outside inspection, check manufacturer's attached validation insert)
(2) Perform Realtime PCR with Standard material (Performance test)
(3) Sequencing of finished product (Efficacy test)
(4) Aliquot 200 ul per vial to avoid repeated thawing and freezing.
(5) Label finished product.
3. dNTP
Package dNTP products with 10 mM of dATP, dCTP, dGTP, dTTP each according to the manufacturer (Qiagen)
(1) Basic testing (outside inspection, check manufacturer's attached validation insert)
(2) Perform Realtime PCR with Standard material (Performance test)
(3) Sequencing of finished product (Efficacy test)
(4) Aliquot 50 ul per vial to avoid repeated thawing and freezing.
(5) Label finished product.
4. RNase free water
Package RNase free water according to the manufacturer (Qiagen).
(1) Basic testing (outside inspection, check manufacturer's attached validation insert)
(2) Perform Realtime PCR with Standard material (Performance test)
(3) Sequencing of finished product (Efficacy test)
(4) Aliquot 1,000 ul per vial to avoid repeated thawing and freezing.
(5) Label finished product.
*Q-Solution in the Qiagen OneStep RT-PCR Kit kit, which usually facilitates amplification of GC-rich templates, is not used in this case.

### Example 8B—Real Time RT PCR Conditions with Primer/Probe/Controls and Individual Steps

Step 1. Specimen/sample processing:
a. RNA should be collected from fresh specimen to ensure suitable RNA quality and quantity.
b. The positive control, NEC (Negative extraction control), and no template (negative) control should be processed simultaneously alongside the specimen.
c. RNA should be extracted using the QIAamp Viral RNA Mini Kit (Qiagen, Qiagen, Hilden, Germany), QIAamp DSP Viral RNA Mini Kit (Qiagen, Hilden, Germany) or Qiagen EZ1Advanced XL Kit (Qiagen, Hilden, Germany) according to the manufacturer's instructions.
d. Extracted RNA (>140 μL) should be eluted in a final volume of 50 μL.
e. Following extraction, the RNA should be used immediately and/or residual RNA stored at −70° C. for later use with correct labeling.
f. When handling the positive control, avoid contamination of the specimen sample as failure to take proper precautions when handling the positive control could result in a false positive result.
   Step 2. Reagent preparation (hereinafter is described based on Rotorgene Q 5-plex HRM RT-PCR machine)
g. Prepare all reagent mixture inside a biological safety cabinet in preparation area. To begin, take out the GG One Step RT-PCR kit contents from freezer and thaw thoroughly at ambient temperature. Vortex and centrifuge briefly. The Enzyme Mix should be kept on ice or cold-block at all times.
h. Take out the GG COVID-19 Internal Control, GG COVID-19 Positive Control and GG COVID-19 Negative Control from freezer and completely thaw them at room temperature. Vortex and centrifuge briefly. Take out the GG COVID-19 probes and primers solutions and place them on ice as well.
i. Calculate the number of reactions (N) that will be included in the test. Be sure to include the no template (negative) control (1 tube), negative extraction control (NEC), the positive control (1 tube), and each specimen. Prepare a volume of master mix as in the table below. It is recommended to prepare 110% of the calculated amount of PCR mix to account for pipetting carryovers. Mix by petting up and down a few times.

| | | Volume/ | Volume for N samples | 110% of volume |
|---|---|---|---|---|
| | Component | test | and 3 controls | (ul) |
| 1 | GG One Step RT-PCR Enzyme mixture | A ul | (N + 3) × A ul | (N + 3) × A × 1.1 |
| 2 | Mixture of primers, probes, TE buffer | B ul | (N + 3) × B ul | (N + 3) × B × 1.1 |
| 3 | GG One Step RT-PCR Enzyme buffer, dNTP, DW | C ul | (N + 3) × C ul | (N + 3) × C × 1.1 |

j. Prepare 72-well plates for real-time RT-PCR based on the estimated number of reactions (N) and prepare the PCR-Mix ingredients as described in above Table.
k. Pipette 204 of PCR-Mix into each well.
l. Cover and transfer the plate into sample processing area.
m. The remaining Reaction Mix and Enzyme Mix must be stored at −20° C. immediately.

### Step 3. Sample Addition:

n. Add 1 uL of the extracted sample RNA to the well pre-filled with reagent mix in the following order: no template (negative) control, Negative extraction control, patient specimen(s), and positive control.
o. Seal the plate and centrifuge at 2000 rpm for 10 seconds to avoid bubbles.
p. Place the plate into real-time RT-PCR system and record the exact location of controls and each specimen.
   Step 4. Real-Time PCR (hereinafter is described based on use of Rotor-Gene Q 5-plex HRM, Qiagen, Germany)
q. 20 μL of reaction mixture containing 1 μL of the RNA is tested
r. Set up and run the Rotor-Gene Q 5-plex HRM Real Time PCR instrument. Refer to its Reference Guideline for detailed instructions. Usually start by double clicking Rotor-GeneQSeries Software 2.1.0 and proceed to Set Up Experiment Properties>Setup the Targets and Samples in Plate Setup>Setup Run Method, then click Run and Start.
s. When setup Experiment Properties, please check the following run settings and choose the correct settings.
   Instrument: 7500 (96 wells)
   Run type: Quantitation—Standard Curve
   Run reagent: TaqMan reagents
   Run mode: Standard
t. When setting up the Targets and Samples, create the following detectors with the quencher set as none. The passive reference must be set as None.

| | Fluoresecent | | | |
|---|---|---|---|---|
| Name of Target Gene | probe ID | Reporter | Quencher | Application |
| RdRp* of SARS-Cov-2 | 17 | FAM | BHQ1 | Most other PCR* excluding Tri-1 |
| ORF1ab of SARS-Cov-2 | 14 | FAM | BHQ1 | Tri-1 |
| | 14~2 | Cy-5 | BHQ2 | Most other PCR |
| N of SARS-Cov-2 (N-1) | 24 | Texas Red | BHQ2 | Triplex-1 |
| N of SARS-Cov-2 (N-2) | 11 | Texas Red | BHQ2 | Quadplex, Triplex-2, Duplex-1/2/3/4 |
| Beta-actin of human | 19 | Hex | BHQ1 | Most other PCR |
| PBGD** of human | 28 | Hex/Vic | BHQ1 | Tri-1 |

u. Set up the plate layout by assigning a unique sample name to each well
v. Assign a Task to each well
   Unknown: for patient samples
   Standard for Positive Control
   NTC for Negative Control
x. Set Run method as followings for PCR amplification and fluorescence detection.
1) Triplex-1/2, Quadplex PCR:

TABLE 29-1
PCR condition-1

| | | | | | Number |
|---|---|---|---|---|---|
| Step | Name | Temperature | | | of cycle |
| 1 | Reverse transcription | 50 C. | 30 | minutes | 1 |
| | (RT) | | | | |
| 2 | Initial denaturation | 95 C. | 15 | minutes | 1 |
| 3 | PCR | 95 C. | 15 | seconds | 40 cycles |
| | | 58 C.* | 40 | seconds | |
| 4 | | 40 C. | 10 | Inutes | |

*Check fluorescence at the final 58 C. step.

2) Single, Duplex PCR:

TABLE 29-2
PCR condition-2

| | | | | Number |
|---|---|---|---|---|
| Step | Name | Temperature | | of cycle |
| 1 | Reverse transcription | 42 C. | 30 minutes | 1 |
| | (RT) | | | |
| 2 | Initial denaturation | 95 C. | 15 minutes | 1 |
| 3 | PCR | 95 C. | 15 seconds | 40 cycles |
| | | 58 C.* | 340 seconds | |
| 4 | | 40 C. | 10 seconds | |

*Check fluorescence at the final 58 C. step.

Step 5. After Real time PCR
When the run is complete, store and analyze the data according to the device manufacturer's instructions. Ct value of each target of each sample is checked and analysis should be performed for each target individually by using manual threshold value setting (threshold value, 0.05). Threshold values should be within the exponential phase of the fluorescence curve and adjusted above the background signal. The procedure you choose to set the threshold value should be used consistently.
   Interpretation method of RT-PCR

### 1. First Step: Examination and Interpretation of Control Results:

The controls for the Real-Time Fluorescent RT-PCR Kit for Detecting COVID-19 are evaluated using the nucleic acid amplification curve and Ct values generated by the RT-PCR system software. The Ct cut-off values are determined using the receiver operator characteristic curves of the tested clinical samples.
The no template (negative) control should provide Ct values at FAM, Texas-Red, Cy-5, and VIC/HEX channels of “0” or “no data available and there should be no sigmoidal amplification curve. If any of the channels are positive, repeat from the RT-PCR step using residual extraction material. If repeat results are not as expected, re-extract and re-test (RT-PCR run) all samples.
The positive and internal control should provide an amplification curve in the FAM, Texas-Red, Cy-5, and VIC/HEX channels that appear to be in a sigmoidal shape.
Cutoff of positivity for COVID-19 markers are obtained by LOD+3SD and is usually set around the Ct value of 40.
The Ct value in the FAM, Texas-Red channel for a valid positive control should be no higher than Cutoff; which is usually 40 (Ct value<40) and there should be a sigmoidal amplification curve. However, the positive control should be negative for IC marker (Ct value<40). If positive results are obtained for COVID-19 markers and/or negative results are obtained for beta actin target, the RT-PCR run is invalid. However, mild (Ct 38-40) positivity in Ct value for IC marker may be seen in Twist RNA, (claim 5-2), and this may be ignored if expected COVID-19 markers are positive. Repeat from the RT-PCR step using residual extraction material. If results are not as expected, re-extract and re-test (RT-PCR run) all samples.
The Ct value in the VIC/HEX channel for a valid internal control should be no higher than 40 (Ct value<40) and there should be a sigmoidal amplification curve. However, the internal control should be negative for COVID-19 marker (Ct value>40) but positive for IC marker (Ct value<40). If positive results are obtained for COVID-19 markers in the internal control or the IC marker tests negative, the RT-PCR run is invalid. Repeat from the RT-PCR step using residual extraction material. If results are not as expected, re-extract and re-test (RT-PCR run) all samples.
The negative extraction control (negative clinical specimen) should be negative for Orflab and N marker but positive for Beta actin (IC marker). If positive results are obtained for N or Orflab targets, the extraction run and the RT-PCR run are invalid and entire process should be repeated using residual patient sample.
2. Second Step: Examination and Interpretation of Patient Specimen Results: Depends on the format; this is explained by the individual section.

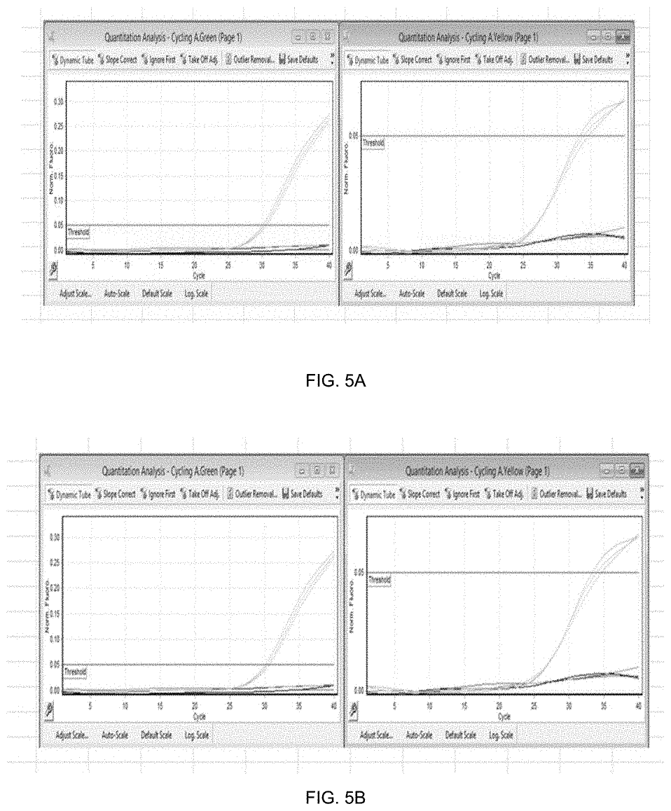

### Example 9—Duplex PCR (See FIG. 5A)

Example 9.1—RdRp and IC (Duplex RT-real time PCR)-1

TABLE 30-1
Duplex-1 (RdRP/beta actin) constituent

| Component | Volume (ul) | |
|---|---|---|
| Positive control RNA | 2 | ul |
| Specific Primer Mix* | 2 | ul |
| Specific Probe Mix* | 2 | ul |
| 2x Onestep RT-PCR buffer | 10 | ul |
| Taq polymerase | 0.5 | ul |
| Onestep RT-PCR Enzyme mix | 0.5 | ul |

| | |
|---|---|
| RNase free Water | Up to 20 ul |

TABLE 30-2
Duplex-1 (RdRP/beta actin) PCR condition

| Step | Temperature | Time |
|---|---|---|
| Reverse transcription | 42° C. | 30 minutes |
| Initial Denaturation | 95° C. | 10 minutes |
| 40 cycles | 95° C. | 15 seconds |
| | 56° C. | 40 seconds |
| Final Extension | 40° C. | 10 seconds |

RdRp and beta-actin duplex RT-real time PCR
RdRp beta-actin RNA concentration: 100 cp/ul
Ct cut off level ≤40.

### Mean Ct: RdRp 30.4 IC 33.95.

TABLE 30-3
LOD validation of Duplex-1(RdRP/IC)

| | FAM | HEX |
|---|---|---|
| Sample | RDRP_Ct | ACT_Ct |
| 2plex_RDRP.ACT_NTC | ND | ND |
| 2plex_RDRP.ACT_NTC | ND | ND |
| 2plex_RDRP.ACT_NTC | ND | ND |
| 2plex_RDRP.ACT_NTC | ND | ND |
| 2plex_RDRP.ACT_10\hat{ }3 | 30.67 | 33.92 |
| 2plex_RDRP.ACT_10\hat{ }3 | 30.12 | 33.3 |
| 2plex_RDRP.ACT_10\hat{ }3 | 30.4 | 34.63 |
| Mean | 30.40 | 33.95 |
| SD | 0.28 | 0.67 |
| CV(%) | 0.90 | 1.96 |

### Result Interpretation

1. First Step: Examination and Interpretation of Control Results are depicted as FIG. 5A.

### Second Step: Examination and Interpretation of Patient Specimen Results:

Assessment of clinical specimen test results should be performed after the positive and no template (negative) controls have been examined and determined to be valid and acceptable. If the controls are not valid, the patient results cannot be interpreted. To be deemed valid, a test must satisfy both the no template (negative) control and positive control requirements noted above. If neither requirement is satisfied, or if only one requirement is satisfied, the test is invalid. In all of these cases, if repeat testing results are not as expected/no new specimen can be obtained, report as “invalid/ask for a new patient specimen”.
I. If the Ct value is less than or equal to 40, it is positive. If the Ct value is greater than 40, it is determined as negative.
II. In case of RdRp positivity, report as “COVID-19 positive”. This may be due to a high concentration of COVID-19 in the specimen.
III. In case of RdRp positivity but beta actin negativity, report as “COVID-19 positive”. This may be due to a high concentration of COVID-19 in the specimen.
V. In case of positive control negativity, or all other cases, proceed from nucleic acid extraction again from leftover sample. If this problem recurs, ask for a new patient specimen. If no new specimen can be obtained, report as “invalid/ask for a new patient specimen”.

TABLE 30-4
Result interpretation of patient results

| | | | | |
|---|---|---|---|---|
| RdRP gene | Positive | Negative | Positive | Negative |
| Beta actin gene | Positive | Positive | Negative | Negative |
| Result | SARS-Cov-2 | SARS-Cov-2 | SARS-Cov-2 | Invalid |
| | detected | not detected | detected | Test |
| Interpretation | COVID-19 | COVID-19 | Positive for | Retest** |
| | Positive | Negative | COVId-19** | |

Example 9.2: Duplex-2 (Orflab/beta actin, See FIG. 5B)

TABLE 31-1
Duplex-2 (Orf1ab/beta actin) constituent

| Component | Volume (ul) | |
|---|---|---|
| Positive control RNA | 2 | ul |
| Specific Primer Mix* | 2 | ul |
| Specific Probe Mix* | 2 | ul |
| 2x Onestep RT-PCR buffer | 10 | ul |
| Taq polymerase | 0.5 | ul |
| Onestep RT-PCR Enzyme mix | 0.5 | ul |

| | |
|---|---|
| RNase free Water | Up to 20 ul |

TABLE 31-2
PCR conditions

| Step | Temperature | Time |
|---|---|---|
| Reverse transcription | 42° C. | 30 minutes |
| Initial Denaturation | 95° C. | 10 minutes |
| 40 cycles | 95° C. | 15 seconds |
| | 56° C. | 40 seconds |
| Final Extension | 40° C. | 10 seconds |

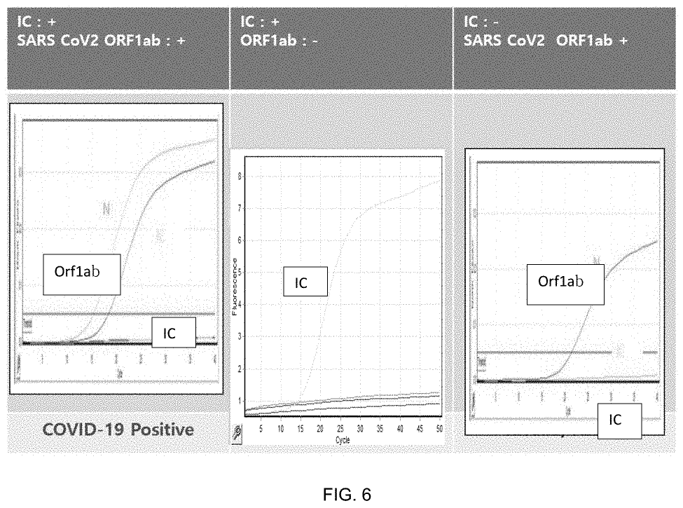

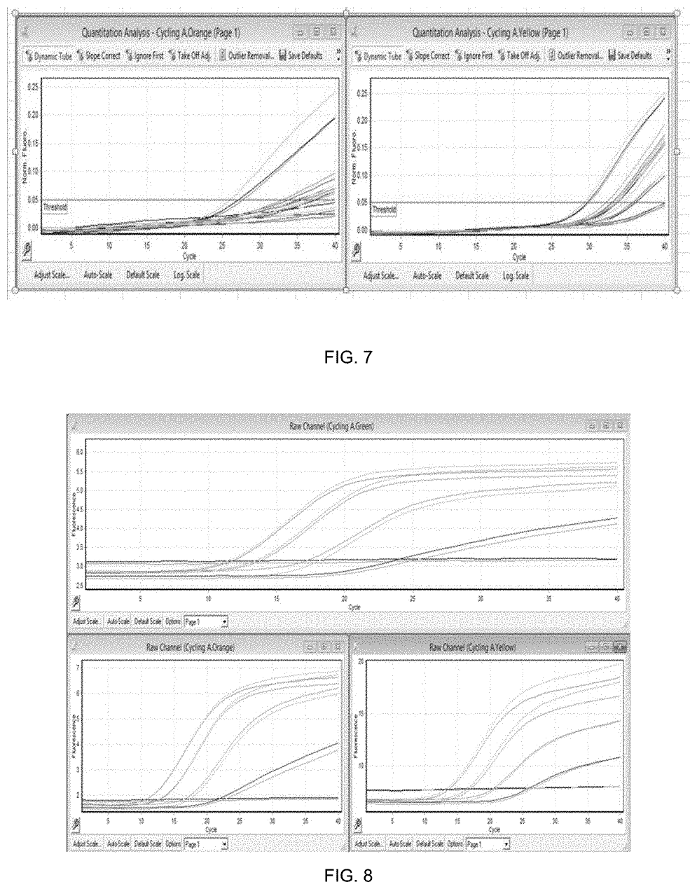

First Step: Examination and Interpretation of Control Results are based on the fluorescence spectroscopy of Duplex-2 used to confirm cases of a positive COVID-19 result for ORFlab and beta-actin duplex RT-real time PCR, where the ORD lab and beta-actin RNA concentration is 100 cp/ul/Ct with a cut off level ≤40. (See FIG. 6.)
Second Step: Examination and Interpretation of Patient Specimen Results involve the following below.
Assessment of clinical specimen test results should be performed after the positive and no template (negative) controls have been examined and determined to be valid and acceptable. If the controls are not valid, the patient results cannot be interpreted. To be deemed valid, a test must satisfy both the no template (negative) control and positive control requirements noted above. If neither requirement is satisfied, or if only one requirement is satisfied, the test is invalid. In all of these cases, if repeat testing results are not as expected/no new specimen can be obtained, report as “invalid/ask for a new patient specimen”.
I. If the Ct value is less than or equal to 40, it is positive. If the Ct value is greater than 40, it is determined as negative.
IL In case of N positivity, report as “COVID-19 positive”. This may be due to a high concentration of COVID-19 in the specimen.
III. In case of N positivity but beta actin negativity, report as “COVID-19 positive”. This may be due to a high concentration of COVID-19 in the specimen.
V. In case of positive control negativity, or all other cases, proceed from nucleic acid extraction again from leftover sample. If this problem recurs, ask for a new patient specimen. If no new specimen can be obtained, report as “invalid/ask for a new patient specimen”.

TABLE 31-3
Result interpretation of patient results

| | | | | |
|---|---|---|---|---|
| ORF1ab gene | Positive | Negative | Positive | Negative |
| Beta actin gene | Positive | Positive | Negative | Negative |
| Result | SARS-Cov-2 | SARS-Cov-2 | SARS-Cov-2 | Invalid |
| | detected | not detected | detected | Test |
| Interpretation | COVID-19 | COV1D-19 | Positive for | Retest** |
| | Positive | Negative | COVId-19** | |

### Example 9.3: N (N-2) and IC(Duplex RT-Real Time PCR)-3

TABLE 32-1
Duplex-3 consituents

| Component | Volume (ul) | |
|---|---|---|
| Positive control RNA | 2 | ul |
| Specific Primer Mix* | 2 | ul |
| Specific Probe Mix* | 2 | ul |
| 2x Onestep RT-PCR buffer | 10 | ul |
| Taq polymerase | 0.5 | ul |
| Onestep RT-PCR Enzyme mix | 0.5 | ul |

| | |
|---|---|
| RNase free Water | Up to 20 ul |

TABLE 32-2
Duplex-3 PCR condition

| Step | Temperature | Time |
|---|---|---|
| Reverse transcription | 42° C. | 30 minutes |
| Initial Denaturation | 95° C. | 10 minutes |
| 40 cycles | 95° C. | 15 seconds |
| | 56° C. | 40 seconds |
| Final Extension | 40° C. | 10 seconds |

N and beta-actin duplex RT-real time PCR
Ct cut off level <40
LoD 5 copy/ul.

TABLE 32-3
Duplex-3 LOD validation

| | Texas Red/N-2 | | | HEX/Beta-actin | | |
|---|---|---|---|---|---|---|
| Sample | 1 | 2 | 3 | 1 | 2 | 3 |
| 2plex_RDRP.ACT_NTC | ND | ND | ND | ND | ND | ND |
| 2plex_RDRP.ACT_NTC | ND | ND | ND | ND | ND | ND |
| 2plex_RDRP.ACT_NTC | ND | ND | ND | ND | ND | ND |
| 2plex_RDRP.ACT_NTC | ND | ND | ND | ND | ND | ND |
| 2plex_RDRP.ACT_100 | 27.77 | 26.1 | 27.19 | 29.93 | 29.96 | 29.72 |
| 2plex_RDRP.ACT_30 | 33.83 | 35.26 | 33.08 | 32.96 | 33.32 | 32.27 |
| 2plex_RDRP.ACT_10 | 37.26 | 35.82 | 34.9 | 33.56 | 33.37 | 33.12 |
| 2plex_RDRP.ACT_5 | 36.63 | 37.62 | 39.46 | 33.72 | 33.73 | 34.41 |
| 2plex_RDRP.ACT_3 | ND | ND | ND | 36.14 | 35.7 | 35.89 |
| 2plex_RDRP.ACT_1 | ND | ND | ND | ND | ND | 39.98 |

First Step: Examination and Interpretation of Control Results are depicted as FIG. 6.

### Second Step: Examination and Interpretation of Patient Specimen Results:

Assessment of clinical specimen test results should be performed after the positive and no template (negative) controls have been examined and determined to be valid and acceptable. If the controls are not valid, the patient results cannot be interpreted. To be deemed valid, a test must satisfy both the no template (negative) control and positive control requirements noted above. If neither requirement is satisfied, or if only one requirement is satisfied, the test is invalid. In all of these cases, if repeat testing results are not as expected/no new specimen can be obtained, report as “invalid/ask for a new patient specimen”.
I. If the Ct value is less than or equal to 40, it is positive. If the Ct value is greater than 40, it is determined as negative.
II. In case of N positivity, report as “COVID-19 positive”. This may be due to a high concentration of COVID-19 in the specimen.
III. In case of N positivity but beta actin negativity, report as “COVID-19 positive”. This may be due to a high concentration of COVID-19 in the specimen.
V. In case of positive control negativity, or all other cases, proceed from nucleic acid extraction again from leftover sample. If this problem recurs, ask for a new patient specimen. If no new specimen can be obtained, report as “invalid/ask for a new patient specimen”.

TABLE 32-4
Duplex-3 Interpretation of patient results

| | | | | |
|---|---|---|---|---|
| N gene (N-2) | Positive | Negative | Positive | Negative |
| Beta actin gene | Positive | Positive | Negative | Negative |
| Result | SARS-Cov-2 | SARS-Cov-2 | SARS-Cov-2 | Invalid |
| | detected | not detected | detected | Test |
| Interpretation | COVID-19 | COVID-19 | Positive for | Retest** |
| | Positive | Negative | COVId-19** | |

### Example 10—Triplex Real Time RT-PCR

### Example 10-1: ORFlab, N, IC (Triplex-1 Real Time RT-PCR)

1. Constituents

TABLE 33-1
Component list and amount of component per reaction

| | Amount/ | Final | | |
|---|---|---|---|---|
| Component | reaction | concentration | Per kit | |
| Template | X ul | 10 ng-1 ug | | |
| F/R Primer Mix | 3 ul | 5 pmole/ul each | 150 | ul |
| Probe Mix | 3 ul | 5 pmole/ul | 150 | ul |
| RT-PCR EnzymeMix | 1 ul | | 50 | ul |
| RT-PCR Buffer (5x) | 4 ul | 1x; 2.5 mM Mg²⁺ | 250 | ul |
| dNTP Mix (10 mM) | 1 ul | 10 mM of each dNTP | 50 | ul |

Positive control: 2 uL/Internal control: 1 uL
Negative control: rest of working solution up to 20 uL total

2. Steps: Refer to Example 8

TABLE 33-2
Triplex-1 PCR fluorescence channel

| Target Name or Detector | Reader | Quencher |
|---|---|---|
| N | FAM | BHQ-1 |
| ORF1a/b | Texas Red (ROX) | BHQ-2 |
| IC (PBGD) | VIC/HEX | BHQ-1 |

TABLE 33-3
Triplex-1 PCR condition

| | | | | Number |
|---|---|---|---|---|
| Step | Name | Temperature | | of cycle |
| 1 | Reverse transcription | 42 C. | 30 minutes | 1 |
| | (RT) | | | |
| 2 | Initial denaturation | 95 C. | 15 minutes | 1 |
| 3 | PCR | 95 C. | 20 seconds | 40 cycles |
| | | 58 C.* | 30 seconds | |
| 4 | | 40 C. | 10 minutes | |

*Check fluorescence at the final 58 C. step.

### Interpretation of Patient Results

Following is a summary of guideline of report and interpretation of result of real time PCR assay by using GoodGene Coronavirus-19 Nucleic Acid Detection Kit.
1) When Ct value of either N or ORFlab or both ORF and N is less than or equal to 40, it is reported as “COVID-19 is detected” under the condition that negative and positive control shows expected result (negative product of ORFlab and N in negative control and positive product of ORFlab and N in positive product in positive control). However, Ct value of internal control is less important in reporting “COVID-19 is detected”.
2) There are two different cases of “COVID-19 is detected”: (a) If the Ct value of patient sample shows that both N and ORRFlab is less than or equal to 40, it is interpreted as “Positive for COVID-19” (b) If Ct value of only 1 of two (either ORFlab or N) is less than or equal to 40, it is interpreted as “Presumptive Positive for COVID-19”
3) When Ct value of both of N and ORFlab is higher than 40, it is reported as “COVID-19 is not detected” and interpreted as “Negative for COVID-19” under the condition that negative and positive control shows expected result (negative product of ORFlab and N in negative control and positive product of ORFlab and N in positive product in positive control).
4) Instances other than above 1), 2) and 3) is reported as “Invalid result” and interpreted as “Repeated Test is required”. When negative and positive control shows do not expected result (positive product of ORFlab and N in negative control or negative product of ORFlab and N in positive), it indicates that all the data in the same batch must be defined as “Invalid Test” and test must be done again by using left over specimens, and if repeated test by using left over specimens shows same result, repeated sampling again is required. If no new specimens can be obtained, report as “invalid/ask for a new patient specimen”.

Refer to the above table to determine the result of real time PCR assay

In case of *, This may be due to a high concentration of COVID-19 in the specimen. If repeat testing with dilution shows same result, interpret as 1)-3) regardless of Pbgd negativity.

In case of **, Additional confirmatory testing may be conducted if necessary, to differentiate between COVID-19 and other SARS-like viruses, for epidemiological purposes or clinical management.

TABLE 33-4
Result interpretation of Triplex-1

| | | | | |
|---|---|---|---|---|
| IC gene | Positive | Positive | Positive | Negative* |
| ORF1ab gene | Both of two | Negative | One out of | Positive or |
| | positive | | two positive | Negative |
| N gene | | Negative | | Positive or |
| | | | | Negative |
| Result | SARS-Cov-2 | SARS-Cov-2 | SARS-Cov-2 | Retest |
| | detected | not detected | detected | |
| Interpretation | COVID-19 | COVID-19 | Presumptive | Retest** |
| | Positive | Negative | Positive for | |
| | | | COVID-19** | |

Below: SARS-Covid-2 RNA copy number 1 × 10\hat{ }5, 1 × 10\hat{ }4, 1 × 10\hat{ }3, 1 × 10\hat{ }2.

Referring to FIG. 8 (a quantitation analysis of Triplex-1), The LoD for ORFlab gene is a 1×10\hat{ }1 copy and N gene 1×10\hat{ }1 copy, at which 100% of both positive controls are positive.
The cutoff for positivity is set at 40 for both positive controls; Thus, if the Ct value is less than or equal to 40, it is positive. If the Ct value is greater than 40, it is determined as negative. Retest if Ct less than or equal to 40 at No template control or either ORFlab or N positive when IC negative.
For IC(Pbgd), cutoff is 35 as mean Ct is higher.

TABLE 33-5
Triplex-1 PCR results of Ct value at each
specimen/concentration close to LOD

| | | concen- | | | | Replicate detection | |
|---|---|---|---|---|---|---|---|
| No. | Target | tration | Mean Ct | SD | CV | (%) | |
| 1 | ORF1ab | 100 | 31.00 | 0.53 | 1.71 | 100 | (20/20) |
| 2 | gene | 10 | 34.38 | 0.46 | 1.34 | 100 | (20/20) |
| 3 | | 1 | 37.70 | 0.80 | 2.12 | 70 | (14/20) |
| 4 | N gene | 100 | 30.87 | 0.31 | 1.00 | 100 | (20/20) |
| 5 | | 10 | 34.75 | 0.52 | 1.50 | 100 | (20/20) |
| 6 | | 1 | 37.21 | 1.47 | 3.95 | 60 | (12/20) |

TABLE 33-6
Triplex-1 PCR LOD Validation with Clinical matrix:

| SARS-COV-2 | | | | Mean Ct | | |
|---|---|---|---|---|---|---|
| RNA | Samples, | Detection Rate | | | | Internal |
| concentration | number | N | ORF1ab | N | ORF1ab | control |
| 2× LOD | 20 | 20/20 | 20/20 | 30.12 | 34.03 | 32.80 |
| 4× LOD | 20 | 20/20 | 20/20 | 28.15 | 33.27 | 31.90 |
| 10× LOD | 5 | 5/5 | 5/5 | 27.83 | 29.75 | 31.72 |
| 20× LOD | 1 | 1/1 | 1/1 | 25.53 | 27.33 | 33.25 |
| 50× LOD | 1 | 1/1 | 1/1 | 24.07 | 26.33 | 33.28 |
| 100× LOD | 1 | 1/1 | 1/1 | 23.56 | 25.72 | 32.79 |
| 200× LOD | 1 | 1/1 | 1/1 | 23.58 | 25.15 | 34.23 |
| 500× LOD | 1 | 1/1 | 1/1 | 23.44 | 24.81 | 33.63 |
| Negative | 60 | 0/64 | 0/64 | | | |

### Example 11-RdRP, N, IC (Triplex RT-Real Time PCR-2)

1. Constituents

TABLE 34-1
Components of COVID-19 Triplex-2
RT Real Time PCR Kit (100 tests)

| | Component | Volume | Voulme/test |
|---|---|---|---|
| SET | (store at −20° C.) | (μl)/100 T | (μl) |
| RT-PCR reagents | RT Enzyme Mix | 50 | 0.5 |
| | RT-PCR Buffer (2x) | 1000 | 10 |
| | Taq polymerase | 50 | 0.5 |
| Primer/Probe/ | Primer Mix (RdRP, N, IC) | 300 | 3 |
| Control Set | Probe Mix (RdRP, N, IC) | 300 | 3 |
| | Positive Control (RdRP, N) | 30 | 2 |
| | Internal Control (IC) | 10 | 1 |
| | RNase-free Water | 300 | Up to 20 |

Positive control: 2 uL/Internal control: 1 uL/Negative control: rest of working solution up to 20 uL total

3. Steps: Refer to Example 8

TABLE 34-2
Triplex-2 fluorescence channel

| Target Name or Detector | Reader | Quencher |
|---|---|---|
| RdRP | FAM | BHQ-1 |
| N-2 | Texas Red (ROX) | BHQ-2 |
| IC (beta actin) | VIC/HEX | BHQ-1 |

TABLE 34-3
Triplex-2 PCR conditions

| | | | | Number |
|---|---|---|---|---|
| Step | Name | Temperature | | of cycle |
| 1 | Reverse transcription | 50 C. | 30 minutes | 1 |
| | (RT) | | | |
| 2 | Initial denaturation | 95 C. | 10 minutes | 1 |
| 3 | PCR | 95 C. | 15 seconds | 40 cycles |
| | | 56 C.* | 40 seconds | |
| 4 | | 40 | 10 seconds | |

4. Triplex-2 Interpretation:

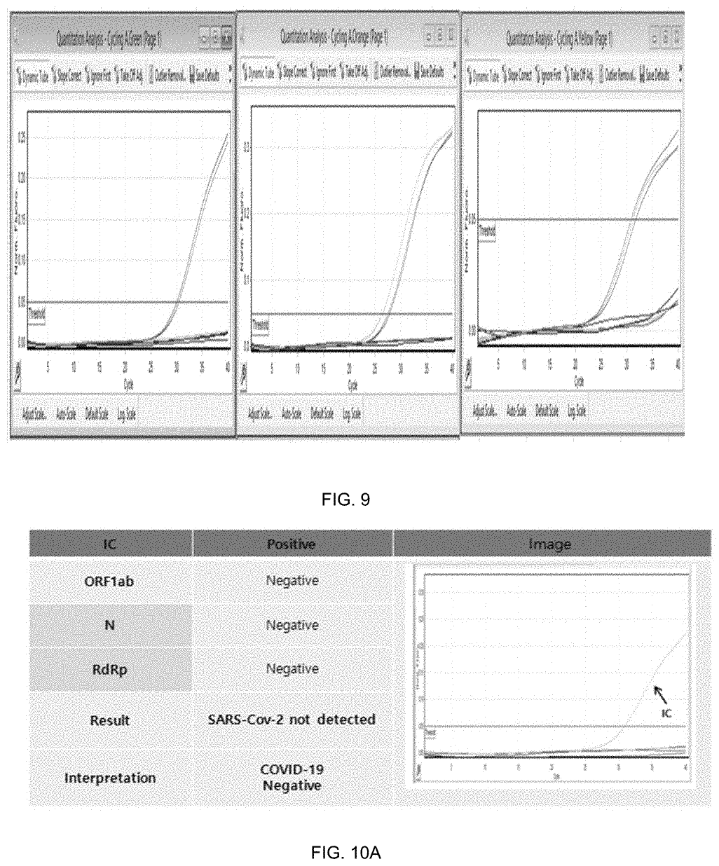

1. First Step: Examination and Interpretation of Control Result are depicted in FIG. 9.
(Same as explained in example 13)

### 2. Second Step: Examination and Interpretation of Patient Specimen Results:

Assessment of clinical specimen test results should be performed after the positive and no template (negative) controls have been examined and determined to be valid and acceptable. If the controls are not valid, the patient results cannot be interpreted. To be deemed valid, a test must satisfy both the no template (negative) control and positive control requirements noted above. If neither requirement is satisfied, or if only one requirement is satisfied, the test is invalid.
I. If the Ct value of patient sample, N and/or RdRP is less than or equal to 40, it is positive. If the Ct value is greater than 40, it is determined as negative.
II. If the Ct value of the IC (Beta actin) is less than or equal to 40, it is positive. If the Ct value is greater than 40, it is determined as negative.
III. Determine the result as shown in the table below.
IV. In case of N and Orflab positivity but internal control negativity, report as “COVID-19 positive”. This may be due to a high concentration of COVID-19 in the specimen.
V. In case of N positivity and Orflab negativity, run RT-PCR again, as there is a high possibility of COVID-19 positivity. If this problem recurs, report as “presumptive COVID-19 positive”.
Additional confirmatory testing may be conducted if necessary, to differentiate between COVID-19 and other SARS-like viruses, for epidemiological purposes or clinical management.
VI. In case of Orflab positivity and N negativity, or all other cases, proceed from nucleic acid extraction again from leftover sample. If this problem recurs, ask for a new patient specimen. If no new specimen can be obtained, report as “invalid/ask for a new patient specimen”.

TABLE 34-4
Triplex-2 Patient result

| | | | | |
|---|---|---|---|---|
| IC gene | Positive | Positive | Positive | Negative |
| RdRP gene | Both of two | Negative | One out of | Positive or |
| | positive | | two positive | Negative |
| N gene | | Negative | | Positive or |
| | | | | Negative |
| Result | SARS-Cov-2 | SARS-Cov-2 | SARS-Cov-2 | Invalid |
| | detected | not detected | detected | Test |
| Inter- | COVID-19 | COVID-19 | Presumptive | Retest** |
| pretation | Positive | Negative | Positive for | |
| | | | COVId-19** | |

TABLE 34-4
Triplex-2 LOD validation

| | FAM | TEXAS RED | HEX |
|---|---|---|---|
| Sample | RDRP_Ct | N_Ct | ACT_Ct |
| 3plex_RDRP.ACT.N_NTC | ND | ND | ND |
| 3plex_RDRP.ACT.N_NTC | ND | ND | ND |
| 3plex_RDRP.ACT.N_NTC | ND | ND | ND |
| 3plex_RDRP.ACT.N_NTC | ND | ND | ND |
| 3plex_RDRP.ACT.N_10\hat{ }3 | 30.17 | 26.47 | 31.21 |
| 3plex_RDRP.ACT.N_10\hat{ }3 | 30.05 | 27.59 | 30.85 |
| 3plex_RDRP.ACT.N_10\hat{ }3 | 30.44 | 27.85 | 31.78 |
| Mean | 30.22 | 27.30 | 31.28 |
| SD | 0.20 | 0.73 | 0.47 |
| CV(%) | 0.66 | 2.69 | 1.50 |

### Example 12—GG COVID-19 Quadplex RT-Real Time PCR Kit Constituents an Steps COVID-19 Quadplex RT-Real Time PCR SOP

TABLE 35-1
Components of GG COVID-19 Quadplex RT Real Time PCR Kit (100 tests, GGCOV-Q-2)

| SET | Component (store at −20° C.) | Volume (μl) |
|---|---|---|
| RT-PCR reagents | RT Enzyme Mix | 50 |
| | RT-PCR Buffer (2×) | 1250 |
| | Hotstart taq Enzyme | 50 |
| Primer/Probe/Control Set | Primer Mix (ORF1ab, N, RdRp, IC) | 400 |
| | Probe Mix (ORF1ab, N, RdRp, IC) | 400 |
| | Positive Control (ORF1ab, N, RdRp) | 60 |
| | Internal Control (IC) | 20 |
| | RNase-free Water | 600 |

TABLE 35-2
Quadplex PCR conditions

| | | | | Number of |
|---|---|---|---|---|
| Step | Name | Temperature | | cycle |
| 1 | Reverse transcription (RT) | 50 C. | 30 minutes | 1 |
| 2 | Initial denaturation | 95 C. | 15 minutes | 1 |
| 3 | PCR | 95 C. | 15 seconds | 40 cycles |
| | | 58 C.* | 40 seconds | |
| 4 | | 40 C. | 10 inutes | |

*Check fluorescence at the final 58 C step.

### Example 13—COVID-19 Quadplex RT-Real Time PCR SOP/Results

### Interpretation of Results

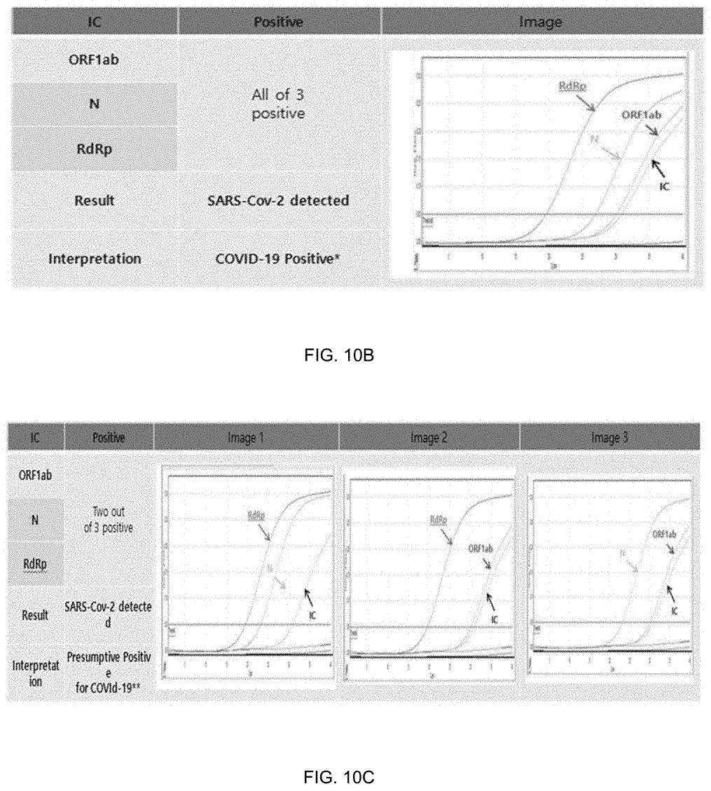

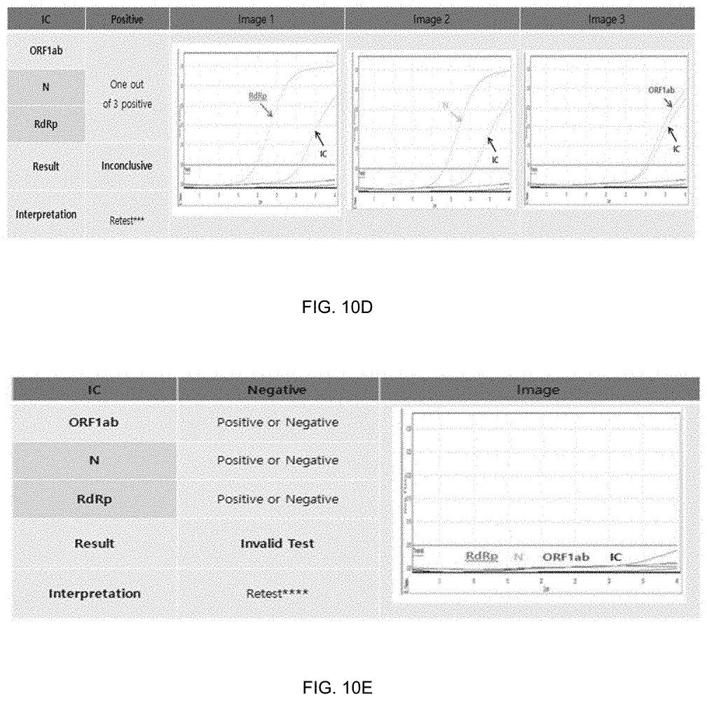

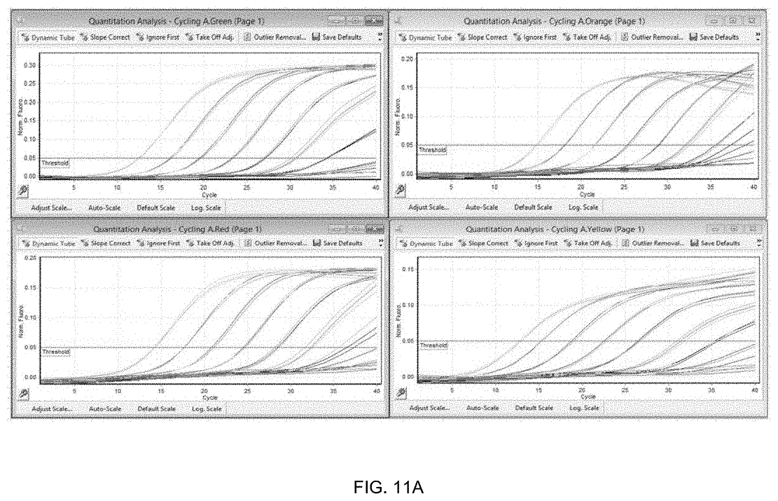

1. First Step: Examination and Interpretation of Control Results based on analysis of the result of reverse transcription real time PCR by using COVID-19 Quadplex RT Real Time PCR Kit (See FIGS. 10A-E).
2. Second Step: Examination and Interpretation of Patient Specimen Results:

Assessment of clinical specimen test results should be performed after the positive, internal negative, and no template controls have been examined and determined to be valid and acceptable (Except in case 4; described in 4)-1.-V.). If the controls are not valid, the patient results cannot be interpreted. To be deemed valid, a test must satisfy all the no template control and positive control requirements noted above. If neither requirement is satisfied, or if only one requirement is satisfied, the test is invalid.
I. If the Ct value is less than or equal to 40, it is positive. If the Ct value is greater than 40 or “0” or “no data available” (no sigmoidal amplification curve), it is determined as negative.
II. Determine the result as shown in the table below.
III. In case of ⅔ positive (N and/or ORFlab and/or RgdP), report as “COVID-19 presumptive positive”.
IV. In case of ⅓ positive (N or ORFlab or RgdP), run RT-PCR again, as there is a high possibility of COVID-19 positivity.
   (if positive in ⅔): report as “COVID-19 presumptive positive”
   (if positive in ⅓, same gene):
      RdRP, N: report as “invalid”
      Orflab: report as “presumptive negative”
V. Additional confirmatory testing may be conducted if necessary, to differentiate between COVID-19 and other SARS-like viruses, for epidemiological purposes or clinical management.
VI. In case of beta-actin (internal control) negativity or positive control negativity, proceed from RT-PCR again, and if results are not as expected, start from nucleic acid extraction again from leftover sample. If results are not as expected, ask for a new patient specimen and report as “invalid”.

TABLE 35-4
Quadplex PCR patient specimen result interpretation

| IC | Positive | Positive | Positive | Positive | Negative |
|---|---|---|---|---|---|
| ORF1ab | All of 3 | Negative | Two out | One out | +/− |
| N | positive | Negative | of 3 positive | of 3 positive | Positive or |
| | | Negative | | | |
| RdRp | | Negative | | | Positive or |
| | | Negative | | | |
| Result | SARS- | SARS- | SARS-Cov-2 | Inconclusive | Invalid Test |
| | Cov-2 | Cov-2 not | detected | | |
| | [symbol] | [symbol] | | | |
| Interpretation | COVID- | COVID- | Presumptive | Retest- | Retest |
| 19 Positive | 19 Negative | | Positive for | >Presumptive | |
| | | | COVId-19 | Negative if | |
| | | | | persistently Orf (+) | |

[symbol]

indicates data missing or illegible when filed

### Example 14—COVID-19 Quadplex RT-Real Time PCR (Limit of Detection, LOD)

Assay performance by using Rotor-Gene Q 5-Plex HRM (Qiagene, Germany) with Rotor-Gene Q Series Software 2.1.0.

### 1.-1) LOD Determination

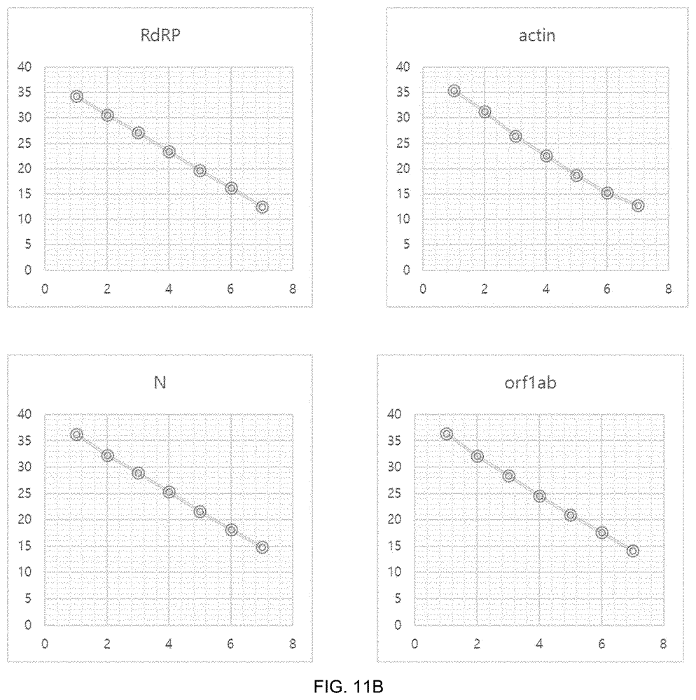

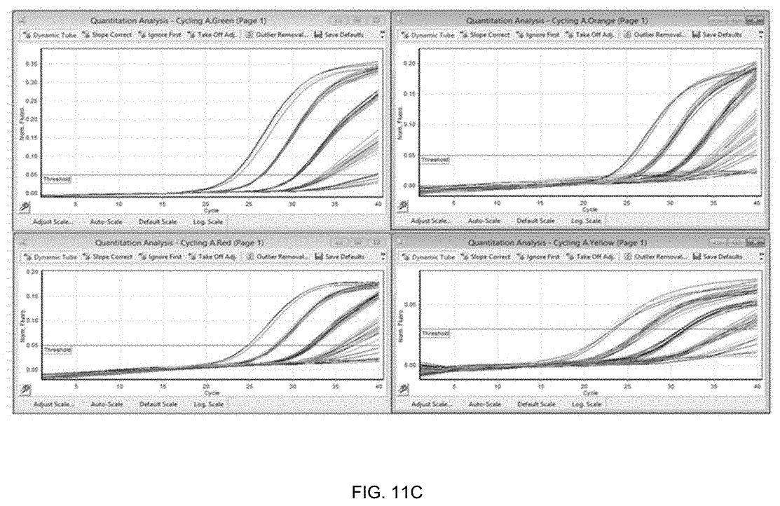

LoD studies determine the lowest detectable concentration of COVID-19 at which approximately ≥95% of all (true positive) replicates test positive. The LoD is determined by limiting dilution studies using characterized samples.
Samples are prepared using pooled clinical oropharyngeal swab specimen matrix collected from 48 healthy Korean adult individuals. The pooled oropharyngeal swab matrix is tested using GG COVID-19 QPlex RT-PCR Kit and confirmed to be negative for SARS-COV-2. In the first part of the study, a total of eight 10-fold dilutions of known concentrations of SARS-CoV-2 viral genome RNA (10\hat{ }9 copies/ml, Total genomic RNA of SARS-Cov-2; MT007544.1 and MN908947.3 Genbank; Twist Bioscience, San Francisco, Calif.) are prepared in negative clinical matrix and processed using the QIAamp Viral RNA Mini Kit (Qiagen, Qiagen, Hilden, Germany). Three replicates per concentration are tested and Ct results obtained.
The results are summarized in the following tables and figures (see FIGS. 11A, 11B, and 11C.).

TABLE 36.1
LoD study results from 10-fold dilution of SARS-Cov-2 RNA (GENOMIC RNA from MT007544.1 and MN908947.3)

| Concentration | RDRP | | | N | | | ORF1ab | | | ACT | | |
|---|---|---|---|---|---|---|---|---|---|---|---|---|
| (copies/ul) | Mean Ct | SD | CV(%) | Mean Ct | SD | CV(%) | Mean Ct | SD | CV(%) | Mean Ct | SD | CV |
| QPlex_10\hat{ }6 | 16.18 | 0.1 | 0.6 | 18.11 | 0.03 | 0.17 | 17.6 | 0.02 | 0.11 | 15.26 | 0.06 | 0.42 |
| QPlex_10\hat{ }5 | 19.66 | 0.11 | 0.58 | 21.64 | 0.03 | 0.12 | 20.95 | 0.15 | 0.72 | 18.77 | 0.16 | 0.87 |
| QPlex_10\hat{ }4 | 23.39 | 0.16 | 0.66 | 25.42 | 0.13 | 0.5 | 24.55 | 0.1 | 0.41 | 22.58 | 0.1 | 0.42 |
| QPlex_10\hat{ }3 | 27.12 | 0.15 | 0.54 | 28.92 | 0.06 | 0.2 | 28.38 | 0.17 | 0.6 | 26.47 | 0.15 | 0.57 |
| QPlex_10\hat{ }2 | 30.56 | 0.4 | 1.32 | 32.32 | 0.24 | 0.75 | 32.08 | 0.47 | 1.48 | 31.31 | 0.48 | 1.52 |
| QPlex_10\hat{ }1 | 34.32 | 0.07 | 0.19 | 36.22 | 0.94 | 2.59 | 36.36 | 0.9 | 2.48 | 35.45 | 0.19 | 0.53 |
| QPlex_10\hat{ }0 | N.D. | | | 38.94 | | | N.D. | | | N.D. | | |
| QPlex 0 | N.D | | | N.D | | | N.D | | | N.D | | |

N.D.: Not detected

0, 1, 3, 10, 30, 100, 300 cp/ulx 20: LoD 10 cp/ul (Table 36.2. and FIG. 11A.)

### Additional Standard Setting of LOD

Analytical sensitivity of GG COVID-19 Quadplex RT Real Time PCR kit was determined in limit of detection studies using cultured SARS-COV-2 virus (BEI, US FDA standard) to determine the lowest concentration at which positive is working for 4 target factors. The original concentration of BEI SARS-Co2 RNA was expected as 5,500 copy/ul. If it was the Viral RNA of 10,000 copy/ml in viral transport medium (VTM), it was extracted 140 ul (1,4000 copy) by Qiagen viral RNA kit. Subsequent BEI SARS-Co2 RNA concentration was used 3.5 ul (140 copy) after isolated as final 35 ul (1,400 copy). The analysis of all specimens was conducted it using three kind Real-Time PCR equipment which are Rotorgene-Q (Qiagen, Hilden, Germany), ABI 7500 (Applied Biosystems Inc., Foster City, Calif.) and CFX96 (Bio-Rad, Hercules, Calif.).

3 and 10-fold dilutions of BEI were prepared with a diluent consisting of a suspension of human A549 cells and VTM. Range-finding was done to determine an estimated LOD. The estimated LoD was defined as the 3×10¹ concentration at which each target (N, ORFlab, RdRp and β-actin) demonstrated positivity (3 out of 3 replicates from data of three Real-Time PCR machines). All assay controls performed as expected, and results of range-finding across all four targets are presented in Tables. and Table.

Table 36-2. Range finding LOD

| | Expectied | | QIAGEN, Rotorgene-Q | | | | ABI, 7500 | | | | BIORAD, CFX96 | | | |
|---|---|---|---|---|---|---|---|---|---|---|---|---|---|---|
| copy/ml | copy number | | N | ORF1ab | RdRp | β-actin | N | ORF1ab | RdRp | β-actin | N | ORF1ab | RdRp | β-actin |
| 1 × 10⁴ | 140 | copy/3.5 ul | 26.24 | 25.55 | 24.53 | 29.45 | 25.95 | 25.86 | 24.67 | 30.31 | 25.65 | 26.06 | 25.21 | 31.12 |
| | | | 26.52 | 25.8 | 24.65 | 29.83 | 26.29 | 25.94 | 24.62 | 30.61 | 25.60 | 25.97 | 25.30 | 30.89 |
| | | | 26.6 | 25.77 | 24.61 | 31.22 | 26.45 | 25.88 | 24.56 | 31.77 | 25.41 | 25.63 | 25.11 | 31.41 |
| 3 × 10³ | 42 | copy/3.5 ul | 27.95 | 26.16 | 24.63 | 31.67 | 27.98 | 27.69 | 26.71 | 31.62 | 27.18 | 27.88 | 27.01 | 33.50 |
| | | | 28.14 | 26.45 | 24.57 | 31.34 | 27.88 | 27 71 | 27.05 | 32.55 | 27.13 | 27.74 | 26.81 | 33.95 |
| | | | 28.03 | 26.81 | 25.08 | 31.61 | 27.83 | 27.69 | 26.52 | 32.11 | 27.11 | 27.76 | 26.42 | 81.22 |
| 1 × 10³ | 14 | copy/3.5 ul | 29.47 | 28.89 | 28.06 | | 29.76 | 29.44 | 28.55 | 34.19 | 29.77 | 30.49 | 30.03 | 33.24 |
| | | | 29.34 | 28.78 | 27.58 | 23.25 | 29.88 | 29.58 | 27.86 | 36.17 | 29.53 | 29.66 | 29.19 | 37.92 |
| | | | 29.4 | 28.84 | 27.73 | 31.47 | 29.94 | 29.56 | 27.73 | 34.42 | 29.80 | 29.74 | 29.39 | 34.72 |
| 3 × 10² | 4.2 | copy/3.5 ul | 31.14 | 31.01 | 29.84 | | 30.82 | 30.28 | 30.14 | | 31.19 | 32.02 | 30.85 | |
| | | | 30.31 | 30.44 | 28.7 | | 31.81 | 31.13 | 30.59 | 37.56 | 31.10 | 31.71 | 30.82 | |
| | | | 30.92 | 30.87 | 29.23 | | 31.49 | 30.73 | 30.67 | | 31.09 | 31.65 | 31.04 | |
| 1 × 10² | 1.4 | copy/3.5 ul | 31.28 | 31.2 | 29.76 | 33.47 | 32.51 | 32.17 | 31.94 | | 32.81 | 32.61 | 32.64 | |
| | | | 32.15 | 32.19 | 30.58 | | 32.28 | 32.05 | 31.71 | | 31.85 | 33.38 | 32.96 | |
| | | | 31.9 | 31.09 | 30.7 | | 32.79 | 31.90 | 31.40 | | 32.85 | 33.94 | 31.67 | |
| 3 × 10¹ | 0.42 | copy/3.5 ul | 34.67 | 35.4 | 35.32 | | 33.29 | 35.08 | 33.63 | 36.17 | 33.32 | 33.28 | 33.79 | |
| | | | 34.45 | 33.68 | 32.66 | | 33.92 | 33.41 | 32.92 | | 33.78 | 33.75 | 33.34 | |
| | | | 33.77 | 34.89 | 33.19 | | 34.16 | 33.34 | 31.94 | | 32.73 | 33.21 | 33.26 | |
| 1 × 10¹ | 0.14 | copy/3.5 ul | 32.78 | 33.99 | 33.72 | | | 36.77 | | | | | | |
| | | | | | | | 34.90 | 36.00 | | | | 35.44 | | |
| | | | | 33.45 | 32.72 | | 34.67 | 34.67 | 33.41 | | | | | |
| 3 × 10⁰ | 0.042 | copy/3.5 ul | | | 32.56 | | | 36.92 | | 33.93 | | | | |
| | | | | | | | | 34.93 | 34.17 | 38.90 | | | | |
| | | | | | 34.93 | | 36.53 | | | | | | | |
| 1 × 10⁰ | 0.014 | copy/3.5 ul | | | | | | | | | | | | |
| | | | | | | | | | 35.70 | 35.16 | | | | |
| | | | | | | | 35.99 | | | | | | | |

NTC

TABLE 36-3
Summary of results of range finding LOD

| | Expectied | | QIAGEN, Rotorgene-Q | | | ABI, 7500 | | | BIORAD, CFX96 | | | |
|---|---|---|---|---|---|---|---|---|---|---|---|---|
| copy/ml | copy number | | N | ORF1ab | RdRp | N | ORF1ab | RdRp | N | ORF1ab | RdRp | |
| 1 × 10⁴ | 140 | copy/3.5ul | 26.45 | 25.71 | 24.60 | 26.23 | 25.89 | 24.62 | 25.55 | 25.89 | 25.21 | Average |
| | | | 0.19 | 0.14 | 0.06 | 0.26 | 0.04 | 0.06 | 0.13 | 0.23 | 0.10 | Standard Deviation [SD] |
| | | | 0.71% | 0.53% | 0.25% | 0.98% | 0.16% | 0.22% | 0.50% | 0.88% | 0.38% | Coefficient of Variation |
| 3 × 10³ | 42 | copy/3.5 ul | 28.04 | 26.47 | 24.76 | 27.90 | 27.70 | 26.76 | 27.14 | 27.79 | 26.75 | |
| | | | 0.10 | 0.33 | 0.28 | 0.08 | 0.01 | 0.27 | 0.04 | 0.08 | 0.30 | |
| | | | 0.34% | 1.23% | 1.13% | 0.28% | 0.04% | 1.01% | 0.13% | 0.27% | 1.12% | |
| 1 × 10³ | 14 | copy/3.5 ul | 29.40 | 28.84 | 27.79 | 29.86 | 29.52 | 28.05 | 29.70 | 29.96 | 29.54 | |
| | | | 0.07 | 0.06 | 0.25 | 0.09 | 0.08 | 0.44 | 0.15 | 0.46 | 0.44 | |
| | | | 0.22% | 0.19% | 0.88% | 0.31% | 0.26% | 1.57% | 0.50% | 1.53% | 1.49% | |
| 3 × 10² | 4.2 | copy/3.5 ul | 30.79 | 30.77 | 29.26 | 31.38 | 30.72 | 30.47 | 31.13 | 31.79 | 30.90 | |
| | | | 0.43 | 0.30 | 0.57 | 0.50 | 0.43 | 0.28 | 0.06 | 0.20 | 0.12 | |
| | | | 1.40% | 0.97% | 1.95% | 1.61% | 1.40% | 0.93% | 0.18% | 0.62% | 0.39% | |
| 1 × 10² | 1.4 | copy/3.5 ul | 31.78 | 31.49 | 30.35 | 32.53 | 32.04 | 31.68 | 32.50 | 33.31 | 32.42 | |
| | | | 0.45 | 0.61 | 0.51 | 0.26 | 0.13 | 0.27 | 0.57 | 0.67 | 0.67 | |
| | | | 1.41% | 1.92% | 1.69% | 0.79% | 0.42% | 0.85% | 1.74% | 2.00% | 2.07% | |
| 3 × 10¹ | 0.42 | copy/3.5 ul | 34.30 | 34.66 | 33.72 | 33.79 | 33.94 | 32.83 | 33.28 | 33.41 | 33.46 | |
| | | | 0.47 | 0.88 | 1.41 | 0.45 | 0.99 | 0.85 | 0.53 | 0.29 | 0.29 | |
| | | | 1.37% | 2.55% | 4.17% | 1.34% | 2.90% | 2.59% | 1.58% | 0.88% | 0.85% | |
| 1 × 10¹ | 0.14 | copy/3.5 ul | 32.78 | 33.72 | 33.22 | 34.79 | 35.81 | 33.41 | | 35.44 | | |
| | | | | 0.38 | 0.71 | 0.16 | 1.06 | | | | | |
| | | | | 1.13% | 2.13% | 0.47% | 2.96% | | | | | |
| 3 × 10⁰ | 0.042 | copy/3.5 ul | | | 33.75 | 36.53 | 35.93 | 34.17 | | | | |
| | | | | | 1.68 | | 1.41 | | | | | |
| | | | | | 4.97% | | 3.94% | | | | | |
| 1 × 10⁰ | 0.014 | copy/3.5 ul | | | | 35.99 | | 35.70 | | | | |

NTC

### 2. Linearity

TABLE 36.2
Linear regression analysis using the SARS-Cov-2 RNA
(BEI, MANASSAS, VA, USA, ATCC: MT135042.1)
(x = concentration of genomic RNA/y = Ct value obtained)

| | |
|---|---|
| RdRP | y = 37.8957 − 3.6239log × (r^{∧}2 = 0.9999) |
| N | y = 39.6071 − 3.5625log × (r^{∧}2 = 0.9996) |
| Orflab | y = 39.5757 − 3.6761log × (r^{∧}2 = 0.9985) |
| Beta-actin | y = 38.6443 − 3.8546log × (r^{∧}2 = 0.9929) |

Result values are transformed to log 10. Linear correlation between the quantification values and corresponding expected values in ranges from 10\hat{ }1 (LLOQ) to 10\hat{ }7 copies/uL (ULOQ) is confirmed. Correlation coefficient (R2) between threshold cycle (Ct) values and quantification values (log 10) in each tested concentration ranged between 0.9929˜0.9999, which verifies the linearity throughout the range tested. The slope of the line in linearity analysis is close to ideal slope of 1 and the intercept is shown to be not significantly different from zero.
3. LoD additional test
Based on the previous results, an additional five 3-fold dilutions of known concentrations of genomic RNA (10\hat{ }9 copies/ml, Total genomic RNA of SARS-Cov-2; MT007544.1 and MN908947.3 Genbank; Twist Bioscience, San Francisco, Calif.) are prepared in negative clinical matrix. Sixty-nine individual extraction replicates are tested in different dilutions. The results are summarized in the table below.

TABLE 36.3
LoD study results from 3-fold dilution of the SARS-Cov-2
RNA (GENOMIC RNA from NR-52281, ATCC; MT135042.1)

| | RdRP | | | | N | | | |
|---|---|---|---|---|---|---|---|---|
| | Mean | SD | CV | | Mean | SD | CV | |
| 300 | 29.14 | 0.12 | 0.40 | 15/15 | 31.45 | 0.21 | 0.67 | 15/15 |
| 100 | 31.92 | 0.24 | 0.75 | 20/20 | 33.60 | 0.37 | 1.11 | 20/20 |
| 30 | 33.65 | 0.36 | 1.06 | 20/20 | 35.75 | 0.77 | 2.14 | 20/20 |
| 10 | 37.28 | 0.97 | 2.60 | 10/10 | 36.51 | 0.66 | 1.81 | 10/10 |
| 1 | | | | 0/4 | | | | 0/4 |
| | ORF1ab | | | | beta-actin | | | |
| | Mean | SD | CV | | Mean | SD | CV | |
| 300 | 30.87 | 0.17 | 0.55 | 15/15 | 29.41 | 0.20 | 0.68 | 15/15 |
| 100 | 33.71 | 0.40 | 1.19 | 20/20 | 31.98 | 0.35 | 1.11 | 20/20 |
| 30 | 35.57 | 0.57 | 1.60 | 20/20 | 34.46 | 0.51 | 1.49 | 20/20 |
| 10 | 37.30 | 0.94 | 2.53 | 10/10 | 37.09 | 0.35 | 0.93 | 10/10 |
| 1 | | | | 0/4 | | | | 0/4 |

The above result indicate that LOD is estimated to be around 10 copies/ml in all cases.

### Example 15—COVID-19 Quadplex RT-Real Time PCR LOD Verification: Analytical Sensitivity

The LoD predicted by probit analysis is further verified by testing 23 extraction replicates of sample at 1× LoD concentration with SARS-CoV-2 viral genome (10\hat{ }9 copies/ml, Total genomic RNA of SARS-Cov-2; MT007544.1 and MN908947.3 Genbank; Twist Bioscience, San Francisco, Calif./acceptable replacement for BEI specimen per FDA EUA standards), which is spiked into oropharyngeal swab matrix according to target ORFlab LOD. 23 replicates of the sample are extracted using QIAamp Viral RNA Mini Kit (Qiagen, Qiagen, Hilden, Germany) and tested using the GG COVID-19 QPlex RT-PCR Kit. The results are summarized in the following table. The LoD is proven acceptable as the detection rate of N, ORFlab, and RdRP, and beta-actin is 100%.

TABLE 37.1
LoD verification results

| | | Detection rate | | | |
|---|---|---|---|---|---|
| Concentration | | N | ORF1ab | RdRP | Beta-actin |
| 1× LOD (10 cp/ul) | | 20/20 | 20/20 | 20/20 | 20/20 |
| | | (100%) | (100%) | (100%) | (100%) |
| Ct value | Mean | 36.79 | 37.47 | 37.50 | 38.04 |
| | SD | 0.52 | 0.90 | 0.75 | 0.57 |
| | CV | 1.41 | 2.42 | 2.00 | 1.51 |
| 3× LOD (30 cp/ul) | | 20/20 | 20/20 | 20/20 | 20/20 |
| | | (100%) | (100%) | (100%) | (100%) |
| Ct value | Mean | 32.91 | 35.12 | 35.49 | 35.01 |
| | SD | 0.40 | 0.81 | 0.79 | 0.38 |
| | CV | 1.20 | 2.30 | 2.21 | 1.08 |

TABLE 37.2
Limit of Detection of the GG COVID-19 with Twist
RNA/BEI specimen (Quadplex assay in viral copy
number of 30, 10 and 3 cp/ul, Replicates = 20)

| 20 | | Concen- | | | | | |
|---|---|---|---|---|---|---|---|
| replicates/ | | tration | Ct value | | | Replicate detection | |
| each | Target | (cp/⊏l) | mean | SD | CV | (%) | |
| 1 | N | 30 | 35.12 | 0.81 | 2.30 | 20/20 | (100) |
| 2 | | 10 | 36.47 | 0.90 | 2.42 | 20/20 | (100) |
| 3 | | 3 | 39.53 | | | 1/20 | (5) |
| 4 | ORF1ab | 30 | 35.49 | 0.79 | 2.21 | 20/20 | (100) |
| 5 | | 10 | 37.5 | 0.75 | 2.00 | 20/20 | (100) |
| 6 | | 3 | 39.35 | | | 3/20 | (15) |
| 7 | RdRp | 30 | 32.91 | 0.40 | 1.20 | 20/20 | (100) |
| 8 | | 10 | 36.79 | 0.52 | 1.41 | 20/20 | (100) |
| 9 | | 3 | N.D. | | | 0/20 | (0) |

N.D.: Not detected
Note) Note)
Cut off level of ORF1ab, N and RdRp is set to Ct ≤ 40 as the maximum value of Ct in which amplification product is observed in this Ct value.

### Example 16—COVID-19 Quadplex RT-Real Time PCR (Analytical Reactivity) (Inclusivity)

BLASTn analysis queries alignments are performed with the SARS-CoV-2 ORFlab, RdRP, and N oligonucleotide primer and probe sequences with all publicly available nucleic acid sequences for 2019-nCoV in GenBank to demonstrate the predicted inclusivity of the GG COVID-19 QPlex RT-PCR Kit. All the alignments show 100% identity to the available 2019-nCoV sequences.

### Example 17—Analytical Specificity and Cross Reactivity

### In Silico Cross-Reactivity

Several organisms are extracted and tested with the GG COVID-19 QPlex Real-Time PCR to demonstrate analytical specificity and exclusivity. Studies are performed with nucleic acids extracted using the_instrument and_ Kit. Nucleic acids are extracted from high titer preparations (typically ≥10\hat{ }5 PFU/mL or ≥10\hat{ }6 CFU/mL). Testing is performed using the QIAGEN OneStep RT-PCR Kit on the Rotorgene Real-Time PCR instrument.
Cross-reactivity of the GG COVID-19 QPlex RT-PCR Kit is evaluated using both in silico analysis and wet testing against normal and pathogenic organisms found in the respiratory tract.
BLASTn analysis queries of the GG COVID-19 QPlex RT-PCR Kit primers and probes are performed against public domain nucleotide sequences with default settings. The database search parameters are as follows:
(i) The nucleotide collection consists of GenBank+EMBL+DDBJ+PDB+RefSeq sequences, but excludes EST, STS, GSS, WGS, TSA, patent sequences as well as phase 0, 1, and 2 HTGS sequences and sequences longer than 100 Mb. The database is non-redundant. Identical sequences have been merged into one entry, while preserving the accession, GI, title and taxonomy information for each entry.
(ii) The match and mismatch scores are 1 and −3, respectively.
(iii) The penalty to create and extend a gap in an alignment is 5 and 2, respectively.
(iv) The search parameters automatically adjusted for short input sequences and the expected threshold is 1000.
   The BLASTn analysis indicated that no organisms, including other related SARS-coronaviruses, exhibit >80% homology to the forward primer, reverse primer, and probe for either the N or the Orflab target.
   RdRP forward primer showed >80% homology to Kenya bat coronaviruses, and RdRP reverse primer showed >80% homology to avian coronaviruses. The probe show no significant homology with human genome, other coronaviruses, or human microflora.
   Combining primers and probe, there is no prediction of potential false positive RT-PCR results.
   The results of the in silico analysis suggest the GG COVID-19 QPlex RT-PCR kit is designed for the specific detection of SARS-CoV-2, with no expected cross reactivity to the human genome, other coronaviruses, or human microflora that would predict potential false positive RT-PCR results.

### Wet Testing

Wet testing against normal and pathogenic organisms of the respiratory tract is performed to confirm the results of the in silico analysis. Each organism identified in the table below is tested in triplicate with the GG COVID-19 QPlex RT-PCR kit at the concentrations indicated. Each replicate is tested with a different reagent lot. All results are negative as shown below.

TABLE 38
Organisms tested for cross-reactivity and results of study

| | | | Catalog | Detected/Replicates | | | Final |
|---|---|---|---|---|---|---|---|
| Virus | Strain | Origin | No. | ORF1ab | N | RdRp | Result |
| Positive Control | SArs-Cov-2, | ATCC | NR-52285 | 31.03 | 32.44 | 31.21 | Positive |
| | Ncov2019- | | | | | | |
| | NCOV/USA- | | | | | | |
| | WA1/202 | | | | | | |
| Human coronavirus | 229E | KU | KBPV-VR-9D | 0/3 | 0/3 | 0/3 | Negative |
| Human coronavirus | OC43 | KU | KBPV-VR-8D | 0/3 | 0/3 | 0/3 | Negative |
| Human coronavirus | HKU1 | ATCC | ATCCVR-3262SD | 0/3 | 0/3 | 0/3 | Negative |
| Human coronavirus | 229E | KU | KBPV-VR-9D | 0/3 | 0/3 | 0/3 | Negative |
| Human coronavirus | NL63 | ATCC | ATCC VR-3263SD | 0/3 | 0/3 | 0/3 | Negative |
| MERS (MERS-CoV) | | ATCC | ATCC VR-3248SD | 0/3 | 0/3 | 0/3 | Negative |
| HumanInfluenza | H1N1 | KU | KBPV-VR-76D | 0/3 | 0/3 | 0/3 | Negative |
| A Virus | | | | | | | |
| HumanInfluenza | H3N2 | KU | KBPV-VR-85D | 0/3 | 0/3 | 0/3 | Negative |
| A Virus | | | | | | | |
| HumanInfluenza | Yamagata | KU | KBPV-VR-34D | 0/3 | 0/3 | 0/3 | Negative |
| B Virus | | | | | | | |
| ADENOVIRUS | Adeno type 71, | KU | KBPV-VR-1D | 0/3 | 0/3 | 0/3 | Negative |
| ENTEROVIRUS | type71 | KU | KBPV-VR-56D | 0/3 | 0/3 | 0/3 | Negative |
| HUMAN | PTV1 | KU | KBPV-VR-64D | 0/3 | 0/3 | 0/3 | Negative |
| PARAINFLUENZA | | | | | | | |
| VIRUS | | | | | | | |
| HUMAN | PTV2 | KU | KBPV-VR-65D | 0/3 | 0/3 | 0/3 | Negative |
| PARAINFLUENZA | | | | | | | |
| VIRUS | | | | | | | |
| HUMAN | PTV3 | KU | KBPV-VR-67D | 0/3 | 0/3 | 0/3 | Negative |
| PARAINFLUENZA | | | | | | | |
| VIRUS | | | | | | | |
| HUMAN | PTV4 | KU | KBPV-VR-69D | 0/3 | 0/3 | 0/3 | Negative |
| PARAINFLUENZA | | | | | | | |
| VIRUS | | | | | | | |
| RESPIRATORY | virus A | KU | KBPV-VR-41D | 0/3 | 0/3 | 0/3 | Negative |
| SYNCYTIAL | | | | | | | |
| VIRUS(RSV) | | | | | | | |
| RESPIRATORY | virus B | KU | KBPV-VR-42D | 0/3 | 0/3 | 0/3 | Negative |
| SYNCYTIAL | | | | | | | |
| VIRUS(RSV) | | | | | | | |
| HUMAN | | KU | KBPV-VR-86D | 0/3 | 0/3 | 0/3 | Negative |
| METAPNEUMO | | | | | | | |
| VIRUS | | | | | | | |
| Rhinovirus A | rhinovirus 1 | KU | KBPV-VR-81D | 0/3 | 0/3 | 0/3 | Negative |
| Rhinovirus A | rhinovirus 7 | KU | KBPV-VR-82D | 0/3 | 0/3 | 0/3 | Negative |
| Rhinovirus A | rhinovirus 8 | KU | KBPV-VR-79D | 0/3 | 0/3 | 0/3 | Negative |
| Rhinovirus A | rhinovirus 21 | KU | KBPV-VR-40D | 0/3 | 0/3 | 0/3 | Negative |
| Rhinovirus B | rhinovirus 14 | KU | KBPV-VR-39D | 0/3 | 0/3 | 0/3 | Negative |
| Rhinovirus B | rhinovirus 42 | KU | KBPV-VR-80D | 0/3 | 0/3 | 0/3 | Negative |
| HUMAN | | ATCC | ATCC VR-3251SD(DNA) | 0/3 | 0/3 | 0/3 | Negative |
| BOCAVIRUS | | | | | | | |
| CHLAMYDIA | | GG | GG CT-91000-043 | 0/3 | 0/3 | 0/3 | Negative |
| TRACHOMATIS | | | | | | | |
| MYCOPLASMA | | GG | GG MP-91000-045 | 0/3 | 0/3 | 0/3 | Negative |
| PNEUMONIAE | | | | | | | |
| Chlamydia | | GG | GG CP-91000-012 | 0/3 | 0/3 | 0/3 | Negative |
| pneumoniae | | | | | | | |
| Haemophilus | | GG | GG HI-91000-023 | 0/3 | 0/3 | 0/3 | Negative |
| influenzae | | | | | | | |
| Mycobacterium | | GG | GG MP-91000-015 | 0/3 | 0/3 | 0/3 | Negative |
| tuberculosis | | | | | | | |
| Human | | GG | GGH PV-90000-101 | 0/3 | 0/3 | 0/3 | Negative |
| papillomavirus | | | | | | | |
| Candida albicans | | GG | GG CA-91000-025 | 0/3 | 0/3 | 0/3 | Negative |

Abbreviations -
KU: KOREA UNIVERSITY, Seoul, Korea;
ATCC: American Tissue Culture Collection

### Example 18—COVID-19 Quadplex RT-Real Time PCR (Interfering Substance Analysis)

The potential interference of the substances listed below are tested in both the presence and absence of COVID-19 RNA with the GG COVID-19 Real-Time PCR Kit. COVID-19 positive samples are prepared by mixing each of the potentially interfering substances with the assay positive control (synthetic COVID-19 ORFlab, RdRP, and N RNA template) at approximately 1×10\hat{ }2 copy/2 uL. All positive and negative samples yield expected results.

TABLE 39-1
Substances Tested for Interference

| | | | |
|---|---|---|---|
| Human blood 5% | Ethanol 0.1% | Nasal spray 0.1% | Mucin 60 ug/mL |

TABLE 39-2
Results

| | | No | Human blood | Ethanol | Nasal spray | Mucin |
|---|---|---|---|---|---|---|
| Target | | interference | 5% | 0.1% | 0.1% | 60 ug/mL |
| ORF1ab | Mean Ct | 32.37 | 31.81 | 32.34 | 31.99 | 31.83 |
| | SD | 0.53 | 1.29 | 0.56 | 0.92 | 0.99 |
| | % Interference | — | 1.730 | 0.093 | 1.174 | 1.668 |
| N | Mean Ct | 32.33 | 32.79 | 32.42 | 32.84 | 32.66 |
| | SD | 0.55 | 0.41 | 0.55 | 0.66 | 0.52 |
| | % Interference | — | −1.4228 | −0.2784 | −1.295 | −1.021 |
| RdRP | Mean Ct | 30.68 | 30.99 | 31.42 | 30.89 | 30.66 |
| | SD | 0.35 | 0.51 | 0.52 | 0.97 | 0.52 |
| | % Interference | — | −1.010 | −2.412 | 1.687 | 0.0652 |

### Example 18—Multiple Instruments for LOD Comparison with CFX96 Touch Real Time PCR Detection System (Bio-Rad, Hercules, Calif., USA)

1. LoD Determination with New PCR Machine
Samples are prepared using pooled clinical oropharyngeal swab specimen matrix collected from 48 healthy Korean adult individuals. The pooled oropharyngeal swab matrix is tested using GG COVID-19 QPlex RT-PCR Kit and confirmed to be negative for SARS-COV-2. In the first part of the study, a total of five 10-fold dilutions of known concentrations of SARS-CoV-2 viral genome RNA (10\hat{ }9 copies/ml, Total genomic RNA of SARS-Cov-2; MT007544.1 and MN908947.3 Genbank; Twist Bioscience, San Francisco, Calif.) are prepared in negative clinical matrix and processed using the QIAamp Viral RNA Mini Kit (Qiagen, Hilden, Germany). Three replicates per concentration are tested and Ct results obtained.
The results are summarized in the following tables.

TABLE 40.1
LoD study results from 3-fold dilution of
SARS-Cov-2 RNA (Twist GENOMIC RNA from MT007544.1
and MN908947.3/CFX96, Biorad):

| | RdRP | | | | N | | | |
|---|---|---|---|---|---|---|---|---|
| | Mean | SD | CV | | Mean | SD | CV | |
| 10\hat{ }4 | 23.34 | 0.31 | 1.33 | 04//04 | 25.66 | 0.21 | 0.84 | 04-Apr |
| 10\hat{ }3 | 26.49 | 0.1 | 0.38 | 20/20 | 28.7 | 0.2 | 0.68 | 20/20 |
| 10\hat{ }2 | 30.51 | 0.12 | 0.38 | 20/20 | 32.38 | 0.2 | 0.63 | 20/20 |
| 10\hat{ }1 | 34.14 | 0.39 | 1.13 | 20/20 | 36.29 | 1.42 | 3.92 | 20/20 |
| 10\hat{ }0 | 39.52 | 0.34 | 0.86 | 02//04 | 39.16 | — | — | 01//04 |
| | ORF1ab | | | | beta-actin | | | |
| | Mean | SD | CV | | Mean | SD | CV | |
| 10\hat{ }4 | 24.92 | 0.25 | 1.02 | 04-Apr | 23.22 | 0.35 | 1.5 | 04//04 |
| 10\hat{ }3 | 28.13 | 0.18 | 0.63 | 20/20 | 27.1 | 0.47 | 1.72 | 20/20 |
| 10\hat{ }2 | 32.24 | 0.26 | 0.82 | 20/20 | 31.69 | 0.38 | 1.2 | 20/20 |
| 10\hat{ }1 | 36.28 | 0.76 | 2.1 | 20/20 | 37.19 | 0.72 | 1.95 | 20/20 |
| 10\hat{ }0 | — | — | — | 0/4 | — | — | — | 0//4 |

### 2. LoD Verification: Analytical Sensitivity

The LoD predicted by probit analysis is further verified by testing 20 extraction replicates of sample at 1× LOD concentration. SARS-CoV-2 viral genome (10\hat{ }9 copies/ml, Total genomic RNA of SARS-Cov-2; MT007544.1 and MN908947.3 Genbank; Twist Bioscience, San Francisco, Calif.) is diluted into oropharyngeal swab matrix according to target ORFlab LOD. 20 replicates of the sample are extracted using QIAamp Viral RNA Mini Kit (Qiagen, Qiagen, Hilden, Germany) and tested using the GG COVID-19 QPlex RT-PCR Kit. The results are summarized in the following table. The LOD is proven acceptable as the detection rate of N, ORFlab, RdRP, and beta-actin is 100%.

TABLE 40.2
LoD verification results (Twist GENOMIC RNA from MT007544.1 and MN908947.3)ICFX-
96, Biorad:

| | | Detection rate | | | |
|---|---|---|---|---|---|
| Concentration | | N | ORF1ab | RdRP | Beta-actin |
| 1× LOD | | 20/20 (100%) | 20/20 (100%) | 20/20 (100%) | 20/20 (100%) |
| Ct value | Mean | 37.17 | 37.33 | 38.18 | 38.50 |
| | SD | 0.77 | 1.09 | 0.96 | 0.87 |
| | CV | 2.07 | 2.92 | 2.51 | 2.27 |

The above result indicated that analytical sensitivity of the GG COVID-19 QPlex RT-PCR Kit is 100% at concentration of 1× LOD (10 copies/ml). The above result indicates that analytical sensitivity of the GoodGene Coronavirus-19 Nucleic Acid Detection Kit is 100% at concentration of 1× LOD when analyzed by CFX96 Touch Real time PCR Detection System (BioRad, Hercules, Calif., USA)
In conclusion, analytical performance assay of the GoodGene coronavirus-19 nucleic acid detection kit by using CFX96 Touch Real time PCR Detection System (BioRad) and Rotor-Gene Q 5-Plex HRM showed same result and the GoodGene coronavirus-19 nucleic acid detection kit show excellent performance in both instrument. The GoodGene coronavirus-19 nucleic acid detection kit show excellent performance also in instruments other than CFX96 Touch Real time PCR Detection System and Rotor-Gene Q 5-Plex FIRM.

### Example 19—COVID-19 Quadplex RT-Real Time PCR Precision (Repeatability and Reproducibility)

### 1.Lot to Lot Reproducibility

Cultured and quantified (2×10² copy) 2 Positive Controls of COVID-19 and 1 internal control are used in the panel. These standard materials are tested by one observer in 3 replicates with 3 different lots of GG COVID-19 Real-Time PCR Kits on different days. Every sample in the test panel is blinded for each operator, run, and device. Lot to Lot reproducibility is deemed acceptable if CV is less than 15%. (CLSI EP 26-A) Lot to Lot variations are as in Test Result 2 in genes ORFlab, N, RdRP, and beta-actin, which is deemed acceptable.

[% Diff=(c2−c1)/{(c1+c2)/2}×100(%)]

### Test Result 41.1. Comparison Table of Ct Results by Lot*

| 1 × 10\hat{ }2 | ORF1ab | | N | | RdRp | | IC | |
|---|---|---|---|---|---|---|---|---|
| copy | Ct ± SD | cv(%) | Ct ± SD | cv(%) | Ct ± SD | cv(%) | Ct ± SD | cv(%) |
| LOT1 | 32.22 ± 0.56 | 1.74 | 31.67 ± 0.42 | 1.33 | 32.33 ± 0.32 | 0.99 | 35.54 ± 0.22 | 0.62 |
| LOT2 | 32.89 ± 0.31 | 0.94 | 31.85 ± 0.12 | 0.38 | 31.97 ± 0.27 | 0.84 | 35.33 ± 0.33 | 0.98 |
| LOT3 | 32.76 ± 0.51 | 1.56 | 31.33 ± 0.32 | 1.02 | 32.12 ± 0.41 | 1.28 | 35.43 ± 0.24 | 0.68 |

*Lot1: GGCOV-Q-2-200424001; Lot 2: GGCOV-Q-2-200424002; Lot 3: GGCOV-Q-2-200424003

### Test Result 41.2. Comparison Table of % Difference in Lots*

| | Mean diff (%) | | | |
|---|---|---|---|---|
| | Orf | N | RdRP | IC |
| Lot1-Lot2 | −2.058 | −0.567 | 1.200 | 0.593 |
| Lot2-Lot3 | 0.396 | 1.646 | −0.468 | −0.283 |
| Lot3-Lot1 | 1.662 | −1.079 | −0.652 | −0.310 |

### 2. Inter-Operator Reproducibility

Cultured and quantified (2×10⁶ copy) stocks of 2 Positive Controls of COVID-19 and 1 negative control are used in the panel. Two different operators test each sample in the panel twice a day for at least 3 days. Each operator run the entire panel twice with the same lot of GG COVID-19 Real-time PCR kit. Every sample in the test panel is blinded for each operator, run, and device. Operator to operator reproducibility is deemed acceptable if CV is less than +/−15% (CLSI EP 12-A2). Operator to operator % Diff ranged from 0.031.21%, which is deemed acceptable (<5%).

[% Diff=(c2−c1)/c1×100(%)]

### Test Result 41.3. Comparison Table of Operator Results by Marker and % Difference

| 1 × 10\hat{ }2 | Operator 1 | | | | Operator 2 | | | | Operator to Operator | | |
|---|---|---|---|---|---|---|---|---|---|---|---|
| copy | Lot 1 | Lot2 | Lot3 | mean ± SD | Lot 1 | Lot2 | Lot3 | mean ± SD | Lot1 | Lot 2 | Lot 3 |
| ORF1ab | 31.88 | 32.3 | 31.92 | 32.03 ± 0.19 | 31.97 | 32.02 | 32.11 | 32.03 ± 0.06 | −0.28 | 0.87 | −0.59 |
| N | 31.47 | 31.56 | 31.54 | 31.52 ± 0.04 | 31.72 | 31.82 | 31.88 | 31.81 ± 0.07 | −0.79 | −0.82 | −1.07 |
| RdRp | 32.02 | 32.31 | 32.21 | 32.18 ± 0.12 | 32.14 | 32.54 | 32.32 | 32.33 ± 0.16 | −0.37 | −0.71 | −0.34 |
| IC | 35.47 | 35.66 | 35.85 | 35.66 ± 0.16 | 35.48 | 35.64 | 35.42 | 35.51 ± 0.09 | −0.03 | 0.06 | 1.21 |

### Example 20—Stability and Storage Conditions

### 1. Storage Conditions

−20±2° C. freezer.
   In order to decide on the optimal shipping and storage conditions of the GG COVID-19 Triplex Real-Time PCR Kit, 1.0×10\hat{ }7 copies/ul concentration levels of the Positive control Standard material (COVID-19) are tested, with three duplicates each differing in the storage temperature (Celcius, Room temperature 20° C., Low −20° C., High 37° C.) of the kit after 2 weeks storage. This experiment is performed with reagent kits from 3 different lot numbers for precision and accuracy.
   The Ct result for the 2 testing conditions room (20° C.) and high (37° C.) starts to deteriorate after 2 weeks, in contrast to the kit that is stored in the low temperature condition (−20° C.; +0.24% increase in Ct value for Orflab, no difference in N, and +0.22% for RdRP). Especially, the high temperature strongly (+173.18% increase in Ct value for Orflab, +180.81% for N, and +164.55%) impacts the kit function, compared to room temperature (+5.00% increase in Ct value for Orflab, +14.00% for N, and +27.88% for RdRP). It is noted that storage in high temperature lowers the Ct to the level where the results would not be reliable if the kit is kept at that temperature. Thus, the optimal temperature requirement of the GG COVID-19 Real-Time PCR Kit is set at −20+/−5° C.
      Test Result 42.1. Comparison Table of Ct Results by Temperature (GENOMIC RNA from MT007544.1 and MN908947.3):

| | ORF1ab | | | | | | | | |
|---|---|---|---|---|---|---|---|---|---|
| | low temp −20° C. | | | room temperature 22° C. | | | high temperature 37° C. | | |
| Timeframe | Mean | SD | CV | Mean | SD | CV | Mean | SD | CV |
| 0 | 12.75 | 0.07 | 0.54% | 12.75 | 0.07 | 0.54% | 12.75 | 0.07 | 0.54% |
| 2 | 12.78 | 0.08 | 0.63% | 14.03 | 0.12 | 0.87% | 35.95 | 0.87 | 2.43% |
| 2 week Ct/ | 100.24% | | | 105.00% | | | 281.89% | | |
| 0 week Ct | | | | | | | | | |
| mean Ct change | 0.24% | | | 5.00% | | | 173.18% | | |
| (%) | | | | | | | | | |
| | N | | | | | | | | |
| | low temp −20° C. | | | room temperature 22° C. | | | high temperature 37° C. | | |
| (Weeks) | Mean | SD | CV | Mean | SD | CV | Mean | SD | CV |
| 0 | 11.57 | 0.07 | 0.34% | 11.57 | 0.07 | 0.34% | 11.57 | 0.07 | 0.34% |
| 2 | 11.57 | 0.04 | 0.57% | 13.19 | 0.13 | 0.98% | 32.49 | 0.48 | 1.48% |
| 2 week Ct/ | 100% | | | 114.00% | | | 280.81% | | |
| 0 week Ct | | | | | | | | | |
| mean Ct change | 0% | | | 14.00% | | | 180.81% | | |
| (%) | | | | | | | | | |
| | RdRp | | | | | | | | |
| | low temp −20° C. | | | room temperature 22° C. | | | high temperature 37° C. | | |
| (Weeks) | Mean | SD | CV | Mean | SD | CV | Mean | SD | CV |
| 0 | 12.68 | 0.08 | 0.60% | 12.68 | 0.08 | 0.60% | 12.68 | 0.08 | 0.60% |
| 2 | 12.71 | 0.08 | 0.65% | 16.22 | 0.18 | 1.10% | 33.55 | 0.65 | 1.95% |
| 2 week Ct/ | 100.22% | | | 127.88% | | | 264.55% | | |
| 0 week Ct | | | | | | | | | |
| mean Ct change | 0.22% | | | 27.88% | | | 164.55% | | |
| (%) | | | | | | | | | |

### 2. Expiration Date

6 months
   “The optimal storage requirement of the GG COVID-19 Real-Time PCR Kit is set at 6 months (30 days once opened). Accelerated aging time results at 4/8 days, corresponding to 3, 6 months show the results below. Avoiding excessive freeze/thaw cycles for reagents is recommended.

$$Accelerated Aging Time ( AAT ) = \frac{Desired Real Time ( RT )}{{Q}_{10} [ ( {T}_{AA} - {T}_{RT} ) \text{/} 10 ]}$$

Accelerated Aging Temperature (TAA); Ambient Temperature (TRT); Q10 (Aging Factor if 10° C. temperature change)

### Test Result 41.2. Comparison Table of Ct Result Based on Calculated Aging Time

| Timeframe | ORF1ab | | N | | RdRP | | IC | |
|---|---|---|---|---|---|---|---|---|
| (Months) | Mean | SD | Mean | SD | Mean | SD | Mean | SD |
| 0 | 16.59 | 6.31 | 13.17 | 0.24 | 13.63 | 0.24 | 13.63 | 0.24 |
| 3 | 14.37 | 0.05 | 13.25 | 0.04 | 14.07 | 0.08 | 14.07 | 0.08 |
| 6 | 14.43 | 0.06 | 13.24 | 0.05 | 14.13 | 0.04 | 14.13 | 0.04 |

3. Stability after Opening Kit (Additional Document B8)

In order to decide on the optimal shipping and storage conditions of the GG COVID-19 Real-Time PCR Kit, Positive control Standard material (COVID-19 ORFlab) and Negative control (DW) are tested, with three duplicates freezing and thawing completely up to 10 times. Acceptable CV is +/−5%. This experiment is performed with reagent kits from 3 different lot numbers for precision and accuracy. The Ct results for 1.0×10\hat{ }2 copies/ul concentration levels of the Positive controls are stable (<5% difference)after freezing/defreezing ×10 times. Negative controls stayed negative in all duplicates.

### Test Result 41.3. Comparison Table of Ct Result Upon Repeat Thaw/Freeze Testing

Negative

| | | control | Positive control | | |
|---|---|---|---|---|---|
| Thaw/freeze | Target | Ct value | Mean Ct | SD | CV |
| ×0 | ORF1ab | ND | 32.14 | 0.14 | 0.43% |
| | N | ND | 31.61 | 0.03 | 0.08% |
| | RdRp | ND | 32.12 | 0.03 | 0.08% |
| | IC | ND | 35.52 | 0.1 | 0.30% |
| ×1 | ORF2ab | ND | 32.11 | 0.28 | 0.88% |
| | N | ND | 31.6 | 0.1 | 0.31% |
| | RdRp | ND | 32.1 | 0.12 | 0.37% |
| | IC | ND | 35.81 | 0.22 | 0.61% |
| ×2 | ORF3ab | ND | 32.37 | 0.29 | 0.91% |
| | N | ND | 31.76 | 0.44 | 1.38% |
| | RdRp | ND | 32.4 | 0.27 | 0.85% |
| | IC | ND | 35.23 | 0.32 | 0.92% |
| ×3 | ORF4ab | ND | 32.45 | 0.18 | 0.55% |
| | N | ND | 31.79 | 0.26 | 0.82% |
| | RdRp | ND | 32.18 | 0.17 | 0.54% |
| | IC | ND | 35.55 | 0.26 | 0.72% |
| ×4 | ORF5ab | ND | 32.21 | 0.15 | 0.47% |
| | N | ND | 31.74 | 0.46 | 1.45% |
| | RdRp | ND | 32.75 | 0.14 | 0.43% |
| | IC | ND | 35.63 | 0.2 | 0.57% |
| ×5 | ORF6ab | ND | 32.43 | 0.24 | 0.73% |
| | N | ND | 31.75 | 0.2 | 0.62% |
| | RdRp | ND | 32.54 | 0.27 | 0.84% |
| | IC | ND | 35.73 | 0.3 | 0.83% |
| ×6 | ORF7ab | ND | 32.51 | 0.27 | 0.82% |
| | N | ND | 31.55 | 0.26 | 0.83% |
| | RdRp | ND | 32.58 | 0.14 | 0.44% |
| | IC | ND | 35.56 | 0.31 | 0.87% |
| ×7 | ORF8ab | ND | 32.58 | 0.35 | 1.08% |
| | N | ND | 31.78 | 0.22 | 0.68% |
| | RdRp | ND | 32.51 | 0.32 | 0.98% |
| | IC | ND | 35.36 | 0.46 | 1.31% |
| ×8 | ORF9ab | ND | 32.38 | 0.29 | 0.90% |
| | N | ND | 31.87 | 0.3 | 0.94% |
| | RdRp | ND | 32.5 | 0.41 | 1.27% |
| | IC | ND | 35.65 | 0.34 | 0.95% |
| ×9 | ORF10ab | ND | 32.26 | 0.2 | 0.63% |
| | N | ND | 31.68 | 0.3 | 0.95% |
| | RdRp | ND | 32.57 | 0.25 | 0.78% |
| | IC | ND | 35.85 | 0.13 | 0.37% |
| ×10 | ORF11ab | ND | 32.44 | 0.19 | 0.58% |
| | N | ND | 31.52 | 0.29 | 0.92% |
| | RdRp | ND | 32.85 | 0.18 | 0.56% |
| | IC | ND | 35.92 | 0.33 | 0.93% |

### Example 22—Clinical Performance Evaluation with BEI Specimen Spiked Clinical Matrix

The performance of The GG COVID-19 QPlex RT-PCR Kit is evaluated using contrived clinical oropharyngeal swabs and nasopharyngeal swabs from healthy patients under IRB approval. In total, 128 healthy individuals with no COVID-19 infection history, no COVID-19 symptoms, and no contact with SARS-CoV-2 infected patients within in 14 days are recruited for the study. Both oropharyngeal swabs and nasopharyngeal swabs are collected from the 128 healthy individuals by one doctor. Samples are immediately frozen at −70° C. until use. Positive samples are prepared from 56 of these negative samples. In total, 56 negative clinical samples and 72 contrived positive clinical samples are tested. The RNA content in these samples ranged from 450 ng to 2.5 ug and OD250/OD280 is estimated to be around 3.0, which means that the sample quality is sufficient.
Positive samples are spiked with SARS-CoV-2 RNA (Total genomic RNA of SARS-Cov-2; MT007544.1 and MN908947.3 Genbank; Twist Bioscience, San Francisco, Calif.) is spiked into 30 of the combined oropharyngeal swabs and the nasopharyngeal swabs at various concentrations (2×LoD, 5×LoD, 10×LoD, 20×LoD, 50×LoD, 100×LoD, 200×LoD, 500×LoD, 1000×LoD), according to the LoD of target RdRP, 10 copies/ml. Of the 56 contrived positive samples, 20 are spiked at concentrations equivalent to 2× and 5× the LoD, 10 are spiked with concentrations equivalent to 10× the LoD, the rest are spiked with 1 each of concentrations equivalent to 20˜1,000× the LoD.
The 128 oropharyngeal and nasopharyngeal samples are tested in a blinded fashion (samples are prepared and capped, then all the tubes are mixed in a box and extracted using GG COVID-19 QPlex kit in a random order. Results of the study are summarized below.

TABLE 41
Clinical Evaluation of nasopharyngeal and oropharyngeal swabs

| Concentration | | | | | | | | |
|---|---|---|---|---|---|---|---|---|
| of SARS-Cov-2 | Detection rate (%) | | | | Ct value (mean +/− SD) | | | |
| genomic RNA | N | ORF1ab | RdRp | IC | N | ORF1ab | RdRp | IC |
| 2× LoD | 20/20 | 20/20 | 20/20 | 20/20 | 35.81 +/− | 33.20 +/− | 31.59 +/− | 32.13 +1− |
| | | | | | 0.43 | 0.25 | 0.25 | 0.32 |
| 5× LoD | 20/20 | 20/20 | 20/20 | 20/20 | 33.03 +/− | 31.42 +/− | 29.25 +/− | 32.43 +/− |
| | | | | | 0.25 | 0.38 | 0.12 | 0.25 |
| 10× LoD | 10/10 | 10/10 | 10/10 | 10/10 | 31.99 +/− | 30.78 +/− | 28.22 +/− | 32.21 +/− |
| | | | | | 0.15 | 0.25 | 0.09 | 0.41 |
| 20× LoD | 1/1 | 1/1 | 1/1 | 1/1 | 29.0 | 29.66 | 28.03 | 32.34 |
| 50× LoD | 1/1 | 1/1 | 1/1 | 1/1 | 28.08 | 28.50 | 27.11 | 32.78 |
| 100× LoD | 1/1 | 1/1 | 1/1 | 1/1 | 26.31 | 26.40 | 26.53 | 32.92 |
| 200× LoD | 1/1 | 1/1 | 1/1 | 1/1 | 25.88 | 25.85 | 25.88 | 32.88 |
| 500× LoD | 1/1 | 1/1 | 1/1 | 1/1 | 24.09 | 23.85 | 22.38 | 32.46 |
| 1000× LoD | 1/1 | 1/1 | 1/1 | 1/1 | 22.49 | 22.21 | 20.05 | 32.65 |
| Negative | 0/72 | 0/72 | 0/72 | 0/72 | N.D. | N.D. | N.D. | N.D. |

Positive percent agreement = 100% (56/56) [95% CI: 94.79~100.00%]
Negative percent agreement = 100% (72/72) [195% CI: 95.92~100.00%]

### Example 23—Clinical Performance Evaluation with Comparison with Previously Proven Commercial Method and Inter-Labaratory Testing

1. Methods and Results

As a general reference, Seegene Allplex RT-PCR (Seegene, South Korea/No. 20-119) is used for Single center, single blinded, randomized, retrospective confirmative study. Seoul Clinical Laboratories carried out the testing utilizing 60 (20 positive and 40 negative) leftover samples out of which 45 are nasopharyngeal and oropharyngeal swab samples and 15 sputum samples, respectively. It is to be noted that Orflab marker in GG QPlex Real Time RT-PCR kit is not tested with the Allplex™2019-nCoV Assay (Seegene), which tests E, RdRP, and N markers.

2. Clinical Subjects

The study included all 60 patients who fitted a World Health Organization (WHO) definition of Person Under Investigation for COVID-19 and are given COVID-19 testing in various clinical settings in Seoul, South Korea.

Briefly, the case definition for COVID-19 PUI is as defined in the CSTE Interim-20-ID-01 Guidance, “Standardized surveillance case definition and national notification for 2019 novel coronavirus disease (COVID-19)”, for additional details on confirmed and probable case requirements: https://cdn.ymaws.com/www.cste.org/resource/resmgr/2020ps/Interim-20-ID-01_COVID-19.pdf

Sample collection period: Mar. 20˜Apr. 27, 2020
Test period: Apr. 27˜Apr. 28, 2020
RNA extraction method: King Fisher Flex, automated, Cat No #5400610 (Thermo Fisher, Waltham, Mass., USA)
Sample type: leftover RNA (>50 ul), −70 C preservation

3. Process as described in the above Examples.

4. Result:

The results from testing Upper Respiratory specimens including 45 Oropharyngeal swabs (P #1-15/N #1-N #30)+15 sputum specimens (P #16-20/N #31-N #40) shown in Table 42 generated a Positive Percent Agreement (PPA): 100.00% (20/20) [95% CI: 98.18-100.00%], and a Negative Percent Agreement (NPA): 100.00% (39/39) [95% CI: 91.24-100.00%], and a Total Percent Agreement (TPA): 100.00% (59/59) [95% CI: 93.89-100.00%].
It is to be noted that there is 1 specimen that is tested negative by GG kit but inconclusive by the Allplex kit; thus, this specimen is excluded in the agreement calculation(drop-out).
It is to be noted that there are 2 negative specimens that only tested positive for Orflab that are considered to be presumptive negative upon repeat testing. There is 1 positive specimen that tested positive initially that are considered to be presumptive negative upon repeat testing.
Positive percent agreement=100% (20/20)
Negative percent agreement=100% (39/39)

TABLE 42.1
Clinical Evaluation of Pharyngeal swabs and Sputum

| Result | Allplex ™ 2019-nCoV Assay Real-time RT-PCR Panel | | | |
|---|---|---|---|---|
| GG COVID-19 Qplex RT-PCR | | | Not | |
| Assay | Detected | Inconclusive | Detected | Total |
| Detected/Positive | 18 | 0 | 0 | 18 |
| presumptive positive | 1 | 0 | 0 | 1 |
| Presumed negative | 0 | 0 | 2 | 2 |
| Invalid/Inconclusive | 0 | 0 | 0 | 0 |
| Negative | 0 | 1 | 38 | 39 |
| Total | 19 | 1 | 40 | 60 |

TABLE 43.2
Clinical Evaluation of Pharyngeal swabs

| Result nasopharyngeal swab | Allplex ™ 2019-nCoV Assay Real-time RT-PCR Panel | | | |
|---|---|---|---|---|
| GG COVID-19 Qplex RT-PCR | | | Not | |
| Assay | Detected | Inconclusive | Detected | Total |
| Detected/Positive | 15 | 0 | 0 | 15 |
| presumptive positive | 0 | 0 | 0 | 0 |
| Presumed negative | 0 | 0 | 2 | 2 |
| Invalid/Inconclusive | 0 | 0 | 0 | 0 |
| Negative | 0 | 0 | 28 | 28 |
| Total | 15 | 0 | 30 | 45 |

TABLE 42.3
Clinical Evaluation of Sputum

| Result-sputum | Allplex ™ 2019-nCoV Assay Real-time RT-PCR Panel | | | |
|---|---|---|---|---|
| GG COVID-19 Qplex RT-PCR | | | Not | |
| Assay | Detected | Inconclusive | Detected | Total |
| Detected/Positive | 3 | 0 | 0 | 3 |
| presumptive positive | 1 | 0 | 0 | 1 |
| Presumed negative | 0 | 0 | 0 | 0 |
| Invalid/Inconclusive | 0 | 0 | 0 | 0 |
| Negative | 0 | 1 | 10 | 11 |
| Total | 4 | 1 | 10 | 15 |

5. Conclusion

This study tess the clinical efficacy of the GG COVID-19 Quadplex Real Time PCR Kit for COVID-19 based on the results of the test using the Allplex™ 2019-nCoV Assay for urgent use for the upper and lower respiratory tract samples.
The match rate evaluation results show 100% positive match rate (95% confidence interval 98.18%-100.00%), negative match rate 100% (95% confidence interval 91.24%-100.00%), and overall match rate 100% (95% confidence interval 93.89%-100.00%), and the consistency evaluation results showed kappa=1, which is higher than the target value 0.8.

6. Market Potential

The COVID-19 virus sequence allows the development of various diagnostic tests as described above. Given the severity of the disease and its rapid global spread, it is highly likely that significant demands for diagnostic tests, therapies and vac-cines to battle against the disease, will arise on a global scale. In addition, the systems and methods contains genetic information which can be applied for clinical and scientific research applications.

### Example 24—Specificity Cross-Reactivity Testing

Specificity/Inclusivity Testing: In Silico Analysis: BLASTn analysis queries of the COVID-19 rRT-PCR assays primers and probes are performed against public domain nucleotide sequences. Sequence homology with related Pathogens and pathogens that are likely to be present in the clinical specimen have been evaluated in silico by sequence alignment to identify the homology between the primers/probe of the assay and the pathogens. BLASTn analysis queries alignments are performed with the SARS-CoV-2 ORFlab, RdRP, and N oligonucleotide primer and probe sequences with all publicly available nucleic acid sequences for 2019-nCoV in GenBank to demonstrate the predicted inclusivity of the GG COVID-19 QPlex RT-PCR Kit. All the alignments show 100% identity to the available 2019-nCoV sequences.

TABLE 43
Primer and probes: reference sequence of target gene and
sequence of primer and probes

| Target | Primer/probe | Sequence (5′→3′) | no | Accession no. |
|---|---|---|---|---|
| ORF1ab | Forward primer | GGGTTTTACACTTAAAAACACAGTC | 25 | MN908947.3 |
| | Reverse primer | GCATCAGCTGACTGAAGCAT | 20 | |
| | Probe | CCGTCTGCGGTATGTGGAAAGGTTATGG | 28 | |
| N | Forward primer | CAACTCCAGGCAGCAGTAGG | 20 | MN908947.3 (n2) |
| | Reverse primer | CCAGACATTTTGCTCTCAAGC | 21 | MT326159.1(n1, n5) |
| | Probe | TTGCTGCTGCTTGACAGATT | 20 | |
| RdRP | Forward primer | CATGTGTGGCGGTTCACTAT | 20 | MN908947.3 |
| | | (Kenya bat corona) | | |
| | Reverse primer | TGTTAAAAACACTATTAGCATAAGCAG | 27 | |
| | | (Avian corona) | | |
| | Probe | CAGGTGGAACCTCATCAGGAGATGC | 25 | |
| Beta | Forward primer | GCACCACACCTTCTACAATGA | 21 | NM_001101.5 |
| actin | Reverse primer | GTCATCTTCTCGCGGTTGGC | 20 | |
| | Probe | CACCCCGTGCTGCTGACCGAGGC | 23 | |

TABLE 44
COVID-19_ORF1ab Assay: (ORF1ab gene: GenBank accession: MN9089473)

| Accession | identity (%) | | | |
|---|---|---|---|---|
| No | F | R | P | Description |
| MT198652. | 100 | 100 | 100 | Severe acute respiratory syndrome coronavirus 2 isolate SARS- |
| 2 | | | | CoV-2/Valencia003/human/2020/ESP, complete genome |
| MT253710. | 100 | 100 | 100 | Severe acute respiratory syndrome coronavirus 2 isolate SARS- |
| 1 | | | | CoV-2/HZ-91/human/2020/CHN, complete genome |
| MT253709. | 100 | 100 | 100 | Severe acute respiratory syndrome coronavirus 2 isolate SARS- |
| 1 | | | | CoV-2/HZ-90/human/2020/CHN, complete genome |
| MT253708. | 100 | 100 | 100 | Severe acute respiratory syndrome coronavirus 2 isolate SARS- |
| 1 | | | | CoV-2/HZ-79/human/2020/CHN, complete genome |
| MT253707. | 100 | 100 | 100 | Severe acute respiratory syndrome coronavirus 2 isolate SARS- |
| 1 | | | | CoV-2/HZ-638/human/2020/CHN, complete genome |
| MT253706. | 100 | 100 | 100 | Severe acute respiratory syndrome coronavirus 2 isolate SARS- |
| 1 | | | | CoV-2/HZ-62/human/2020/CHN, complete genome |
| MT253705. | 100 | 100 | 100 | Severe acute respiratory syndrome coronavirus 2 isolate SARS- |
| 1 | | | | CoV-2/HZ-60/human/2020/CHN, complete genome |
| MT253704. | 100 | 100 | 100 | Severe acute respiratory syndrome coronavirus 2 isolate SARS- |
| 1 | | | | CoV-2/HZ-576/human/2020/CHN, complete genome |
| MT253703. | 100 | 100 | 100 | Severe acute respiratory syndrome coronavirus 2 isolate SARS- |
| 1 | | | | CoV-2/HZ-551/human/2020/CHN, complete genome |
| MT253702. | 100 | 100 | 100 | Severe acute respiratory syndrome coronavirus 2 isolate SARS- |
| 1 | | | | CoV-2/HZ-49/human/2020/CHN, complete genome |
| MT253701. | 100 | 100 | 100 | Severe acute respiratory syndrome coronavirus 2 isolate SARS- |
| 1 | | | | CoV-2/HZ-48/human/2020/CHN, complete genome |
| MT253700. | 100 | 100 | 100 | Severe acute respiratory syndrome coronavirus 2 isolate SARS- |
| 1 | | | | CoV-2/HZ-481/human/2020/CHN, complete genome |
| MT253699. | 100 | 100 | 100 | Severe acute respiratory syndrome coronavirus 2 isolate SARS- |
| 1 | | | | CoV-2/HZ-477/human/2020/CHN, complete genome |
| MT253698. | 100 | 100 | 100 | Severe acute respiratory syndrome coronavirus 2 isolate SARS- |
| 1 | | | | CoV-2/HZ-185/human/2020/CHN, complete genome |
| MT253697. | 100 | 100 | 100 | Severe acute respiratory syndrome coronavirus 2 isolate SARS- |
| 1 | | | | CoV-2/HZ-178/human/2020/CHN, complete genome |
| MT253696. | 100 | 100 | 100 | Severe acute respiratory syndrome coronavirus 2 isolate SARS- |
| 1 | | | | CoV-2/HZ-162/human/2020/CHN, complete genome |
| MT251980. | 100 | 100 | 100 | Severe acute respiratory syndrome coronavirus 2 isolate SARS- |
| 1 | | | | CoV-2/WA-UW242/human/2020/USA, complete genome |
| MT251979. | 100 | 100 | 100 | Severe acute respiratory syndrome coronavirus 2 isolate SARS- |
| 1 | | | | CoV-2/WA-UW241/human/2020/USA, complete genome |
| MT251978. | 100 | 100 | 100 | Severe acute respiratory syndrome coronavirus 2 isolate SARS-Co |
| 1 | | | | UW235/human/2020/USA, complete genome |
| MT251977. | 100 | 100 | 100 | Severe acute respiratory syndrome coronavirus 2 isolate SARS- |
| 1 | | | | CoV-2/WA-UW234/human/2020/USA, complete genome |
| MT251976. | 100 | 100 | 100 | Severe acute respiratory syndrome coronavirus 2 isolate SARS- |
| 1 | | | | CoV-2/WA-UW240/human/2020/USA, complete genome |
| MT251975. | 100 | 100 | 100 | Severe acute respiratory syndrome coronavirus 2 isolate SARS- |
| 1 | | | | CoV-2/WA-UW239/human/2020/USA, complete genome |
| MT251974. | 100 | 100 | 100 | Severe acute respiratory syndrome coronavirus 2 isolate SARS- |
| 1 | | | | CoV-2/WA-UW238/human/2020/USA, complete genome |
| MT251973. | 100 | 100 | 100 | Severe acute respiratory syndrome coronavirus 2 isolate SARS- |
| 1 | | | | CoV-2/WA-UW237/human/2020/USA, complete genome |
| MT251972. | 100 | 100 | 100 | Severe acute respiratory syndrome coronavirus 2 isolate SARS- |
| 1 | | | | CoV-2/WA-UW236/human/2020/USA, complete genome |
| MT184913. | 100 | 100 | 100 | Severe acute respiratory syndrome coronavirus 2 isolate 2019- |
| 1 | | | | nCoV/USA-CruiseA-26/2020, complete genome |
| MT184912. | 100 | 100 | 100 | Severe acute respiratory syndrome coronavirus 2 isolate 2019- |
| 1 | | | | nCoV/USA-CruiseA-25/2020, complete genome |
| MT184911. | 100 | 100 | 100 | Severe acute respiratory syndrome coronavirus 2 isolate 2019- |
| 1 | | | | nCoV/USA-CruiseA-24/2020, complete genome |
| MT184910. | 100 | 100 | 100 | Severe acute respiratory syndrome coronavirus 2 isolate 2019- |
| 1 | | | | nCoV/USA-CruiseA-23/2020, complete genome |
| MT184909. | 100 | 100 | 100 | Severe acute respiratory syndrome coronavirus 2 isolate 2019- |
| 1 | | | | nCoV/USA-CruiseA-22/2020, complete genome |
| MT184908. | 100 | 100 | 100 | Severe acute respiratory syndrome coronavirus 2 isolate 2019- |
| 1 | | | | nCoV/USA-CruiseA-21/2020, complete genome |
| MT184907. | 100 | 100 | 100 | Severe acute respiratory syndrome coronavirus 2 isolate 2019- |
| 1 | | | | nCoV/USA-CruiseA-19/2020, complete genome |
| MN996532 | 100 | 100 | 100 | Bat coronavirus RaTG13, complete genome |
| .1 | | | | |
| MT246490. | 100 | 100 | 100 | Severe acute respiratory syndrome coronavirus 2 isolate SARS- |
| 1 | | | | CoV-2/WA-UW233/human/2020/USA, complete genome |
| MT246489. | 100 | 100 | 100 | Severe acute respiratory syndrome coronavirus 2 isolate SARS- |
| 1 | | | | CoV-2/WA-UW232/human/2020/USA, complete genome |
| MT246488. | 100 | 100 | 100 | Severe acute respiratory syndrome coronavirus 2 isolate SARS- |
| 1 | | | | CoV-2/WA-UW231/human/2020/USA, complete genome |
| MT246487. | 100 | 100 | 100 | Severe acute respiratory syndrome coronavirus 2 isolate SARS- |
| 1 | | | | CoV-2/WA-UW230/human/2020/USA, complete genome |
| MT246486. | 100 | 100 | 100 | Severe acute respiratory syndrome coronavirus 2 isolate SARS- |
| 1 | | | | CoV-2/WA-UW229/human/2020/USA, complete genome |
| MT246485. | 100 | 100 | 100 | Severe acute respiratory syndrome coronavirus 2 isolate SARS- |
| 1 | | | | CoV-2/WA-UW228/human/2020/USA, complete genome |
| MT246484. | 100 | 100 | 100 | Severe acute respiratory syndrome coronavirus 2 isolate SARS- |
| 1 | | | | CoV-2/WA-UW227/human/2020/USA, complete genome |
| MT246483. | 100 | 100 | 100 | Severe acute respiratory syndrome coronavirus 2 isolate SARS- |
| 1 | | | | CoV-2/WA-UW226/human/2020/USA, partial genome |
| MT246482. | 100 | 100 | 100 | Severe acute respiratory syndrome coronavirus 2 isolate SARS- |
| 1 | | | | CoV-2/WA-UW225/human/2020/USA, complete genome |
| MT246481. | 100 | 100 | 100 | Severe acute respiratory syndrome coronavirus 2 isolate SARS- |
| 1 | | | | CoV-2/WA-UW224/human/2020/USA, complete genome |
| MT246480. | 100 | 100 | 100 | Severe acute respiratory syndrome coronavirus 2 isolate SARS- |
| 1 | | | | CoV-2/WA-UW223/human/2020/USA, complete genome |
| MT246479. | 100 | 100 | 100 | Severe acute respiratory syndrome coronavirus 2 isolate SARS- |
| 1 | | | | CoV-2/WA-UW222/human/2020/USA, complete genome |
| MT246478. | 100 | 100 | 100 | Severe acute respiratory syndrome coronavirus 2 isolate SARS- |
| 1 | | | | CoV-2/WA-UW221/human/2020/USA, complete genome |
| MT246477. | 100 | 100 | 100 | Severe acute respiratory syndrome coronavirus 2 isolate SARS- |
| 1 | | | | CoV-2/WA-UW220/human/2020/USA, complete genome |
| MT246476. | 100 | 100 | 100 | Severe acute respiratory syndrome coronavirus 2 isolate SARS- |
| 1 | | | | CoV-2/WA-UW219/human/2020/USA, complete genome |
| MT246475. | 100 | 100 | 100 | Severe acute respiratory syndrome coronavirus 2 isolate SARS- |
| 1 | | | | CoV-2/WA-UW218/human/2020/USA, complete genome |
| MT246474. | 100 | 100 | 100 | Severe acute respiratory syndrome coronavirus 2 isolate SARS- |
| 1 | | | | CoV-2/WA-UW217/human/2020/USA, complete genome |
| MT246473. | 100 | 100 | 100 | Severe acute respiratory syndrome coronavirus 2 isolate SARS- |
| 1 | | | | CoV-2/WA-UW216/human/2020/USA, complete genome |
| MT246472. | 100 | 100 | 100 | Severe acute respiratory syndrome coronavirus 2 isolate SARS- |
| 1 | | | | CoV-2/WA-UW215/human/2020/USA, complete genome |
| MT246471. | 100 | 100 | 100 | Severe acute respiratory syndrome coronavirus 2 isolate SARS- |
| 1 | | | | CoV-2/WA-UW214/human/2020/USA, complete genome |
| MT246470. | 100 | 100 | 100 | Severe acute respiratory syndrome coronavirus 2 isolate SARS- |
| 1 | | | | CoV-2/WA-UW213/human/2020/USA, complete genome |
| MT246469. | 100 | 100 | 100 | Severe acute respiratory syndrome coronavirus 2 isolate SARS- |
| 1 | | | | CoV-2/WA-UW212/human/2020/USA, complete genome |
| MT246468. | 100 | 100 | 100 | Severe acute respiratory syndrome coronavirus 2 isolate SARS- |
| 1 | | | | CoV-2/WA-UW211/human/2020/USA, complete genome |
| MT246467. | 100 | 100 | 100 | Severe acute respiratory syndrome coronavirus 2 isolate SARS- |
| 1 | | | | CoV-2/WA-UW210/human/2020/USA, complete genome |
| MT246466. | 100 | 100 | 100 | Severe acute respiratory syndrome coronavirus 2 isolate SARS- |
| 1 | | | | CoV-2/WA-UW209/human/2020/USA, complete genome |
| MT246465. | 100 | 100 | 100 | Severe acute respiratory syndrome coronavirus 2 isolate SARS- |
| 1 | | | | CoV-2/WA-UW208/human/2020/USA, partial genome |
| MT246464. | 100 | 100 | 100 | Severe acute respiratory syndrome coronavirus 2 isolate SARS- |
| 1 | | | | CoV-2/WA-UW207/human/2020/USA, complete genome |
| MT246463. | 100 | 100 | 100 | Severe acute respiratory syndrome coronavirus 2 isolate SARS- |
| 1 | | | | CoV-2/WA-UW206/human/2020/USA, partial genome |
| MT246462. | 100 | 100 | 100 | Severe acute respiratory syndrome coronavirus 2 isolate SARS- |
| 1 | | | | CoV-2/WA-UW205/human/2020/USA, complete genome |
| MT246461. | 100 | 100 | 100 | Severe acute respiratory syndrome coronavirus 2 isolate SARS- |
| 1 | | | | CoV-2/WA-UW204/human/2020/USA, complete genome |
| MT246460. | 100 | 100 | 100 | Severe acute respiratory syndrome coronavirus 2 isolate SARS- |
| 1 | | | | CoV-2/WA-UW203/human/2020/USA, complete genome |
| MT246459. | 100 | 100 | 100 | Severe acute respiratory syndrome coronavirus 2 isolate SARS- |
| 1 | | | | CoV-2/WA-UW202/human/2020/USA, complete genome |
| MT246458. | 100 | 100 | 100 | Severe acute respiratory syndrome coronavirus 2 isolate SARS- |
| 1 | | | | CoV-2/WA-UW201/human/2020/USA, complete genome |
| MT246457. | 100 | 100 | 100 | Severe acute respiratory syndrome coronavirus 2 isolate SARS- |
| 1 | | | | CoV-2/WA-UW200/human/2020/USA, complete genome |
| MT246456. | 100 | 100 | 100 | Severe acute respiratory syndrome coronavirus 2 isolate SARS- |
| 1 | | | | CoV-2/WA-UW199/human/2020/USA, complete genome |
| MT246455. | 100 | 100 | 100 | Severe acute respiratory syndrome coronavirus 2 isolate SARS- |
| 1 | | | | CoV-2/WA-UW198/human/2020/USA, complete genome |
| MT246454. | 100 | 100 | 100 | Severe acute respiratory syndrome coronavirus 2 isolate SARS- |
| 1 | | | | CoV-2/WA-UW197/human/2020/USA, complete genome |
| MT246453. | 100 | 100 | 100 | Severe acute respiratory syndrome coronavirus 2 isolate SARS- |
| 1 | | | | CoV-2/WA-UW196/human/2020/USA, complete genome |
| MT246452. | 100 | 100 | 100 | Severe acute respiratory syndrome coronavirus 2 isolate SARS- |
| 1 | | | | CoV-2/WA-UW195/human/2020/USA, complete genome |
| MT246451. | 100 | 100 | 100 | Severe acute respiratory syndrome coronavirus 2 isolate SARS- |
| 1 | | | | CoV-2/WA-UW194/human/2020/USA, complete genome |
| MT246450. | 100 | 100 | 100 | Severe acute respiratory syndrome coronavirus 2 isolate SARS- |
| 1 | | | | CoV-2/WA-UW193/human/2020/USA, complete genome |
| MT246449. | 100 | 100 | 100 | Severe acute respiratory syndrome coronavirus 2 isolate SARS- |
| 1 | | | | CoV-2/WA-UW192/human/2020/USA, complete genome |
| MT240479. | 100 | 100 | 100 | Severe acute respiratory syndrome coronavirus 2 isolate SARS- |
| 1 | | | | CoV-2/Gilgitl/human/2020/PAK, complete genome |
| MT233523. | 100 | 100 | 100 | Severe acute respiratory syndrome coronavirus 2 isolate SARS- |
| 1 | | | | CoV-2/Valencia8/human/2020/ESP, complete genome |
| MT233522. | 100 | 100 | 100 | Severe acute respiratory syndrome coronavirus 2 isolate SARS- |
| 1 | | | | CoV-2/Valencia7/human/2020/ESP, complete genome |
| MT233521. | 100 | 100 | 100 | Severe acute respiratory syndrome coronavirus 2 isolate SARS- |
| 1 | | | | CoV-2/Valencia6/human/2020/ESP, partial genome |
| MT233520. | 100 | 100 | 100 | Severe acute respiratory syndrome coronavirus 2 isolate SARS- |
| 1 | | | | CoV-2/Valencia4/human/2020/ESP, partial genome |
| MT233519. | 100 | 100 | 100 | Severe acute respiratory syndrome coronavirus 2 isolate SARS- |
| 1 | | | | CoV-2/Valencia5/human/2020/ESP, complete genome |
| MT226610. | 100 | 100 | 100 | Severe acute respiratory syndrome-related coronavirus isolate |
| 1 | | | | SARS-CoV-2/KMS1/human/2020/CHN, complete genome |
| MT020781. | 100 | 100 | 100 | Severe acute respiratory syndrome coronavirus 2 isolate nCoV-FIN- |
| 2 | | | | 29-Jan-2020, partial genome |
| MT198653. | 100 | 100 | 100 | Severe acute respiratory syndrome coronavirus 2 isolate SARS- |
| 1 | | | | CoV-2/Valencia001/human/2020/ESP, partial genome |
| MT198651. | 100 | 100 | 100 | Severe acute respiratory syndrome coronavirus 2 isolate SARS- |
| 1 | | | | CoV-2/Valencia002/human/2020/ESP, partial genome |
| MT192773. | 100 | 100 | 100 | Severe acute respiratory syndrome coronavirus 2 isolate SARS- |
| 1 | | | | CoV-2/nCoV-19-025/human/2020/VNM, complete genome |
| MT192772. | 100 | 100 | 100 | Severe acute respiratory syndrome coronavirus 2 isolate SARS- |
| 1 | | | | CoV-2/nCoV-19-01S/human/2020/VNM, complete genome |
| MT192765. | 100 | 100 | 100 | Severe acute respiratory syndrome coronavirus 2 isolate SARS- |
| 1 | | | | CoV-2/PC00101P/human/2020/USA, complete genome |
| MT192759. | 100 | 100 | 100 | Severe acute respiratory syndrome coronavirus 2 isolate SARS- |
| 1 | | | | CoV-2/CGMH-CGU-01/human2020/TWN, complete genome |
| MT188341. | 100 | 100 | 100 | Severe acute respiratory syndrome coronavirus 2 isolate USA/MN1- |
| 1 | | | | MDH1/2020, complete genome |
| MT188340. | 100 | 100 | 100 | Severe acute respiratory syndrome coronavirus 2 isolate USA/MN2- |
| 1 | | | | MDH2/2020, complete genome |
| MT188339. | 100 | 100 | 100 | Severe acute respiratory syndrome coronavirus 2 isolate USA/MN3- |
| 1 | | | | MDH3/2020, complete genome |
| MT123293. | 100 | 100 | 100 | Severe acute respiratory syndrome coronavirus 2 isolate SARS- |
| 2 | | | | CoV-2/IQTC03/human/2020/CHN, complete genome |
| MT123292. | 100 | 100 | 100 | Severe acute respiratory syndrome coronavirus 2 isolate SARS- |
| 2 | | | | CoV-2/IQTC04/human/2020/CHN, complete genome |
| MT123291. | 100 | 100 | 100 | Severe acute respiratory syndrome coronavirus 2 isolate SARS- |
| 2 | | | | CoV-2/IQTCO2/human/2020/CHN, complete genome |
| MT093631. | 100 | 100 | 100 | Severe acute respiratory syndrome coronavirus 2 isolate SARS- |
| 2 | | | | CoV-2/WH-09/human/2020/CHN, complete genome |
| JX993988. | 100 | 100 | 96.88 | Bat coronavirus Cp/Yunnan2011, complete genome |
| 1 | | | | |
| NC04551 | 100 | 100 | 100 | Wuhan seafood market pneumonia virus isolate Wuhan-Hu-1, |
| 2.2 | | | | complete genome |
| LR757995. | 100 | 100 | 100 | Wuhan seafood market pneumonia virus genome assembly, |
| 1 | | | | chromosome: whole_genome |
| LR757996. | 100 | 100 | 100 | Wuhan seafood market pneumonia virus genome assembly, chromosome:w |
| 1 | | | | hole_genome |

TABLE 45
COVID-19 N Assay: (N gene; GenBank accession: MN908947.3)

| Accession | identity (%) | | | |
|---|---|---|---|---|
| No | F | R | P | Description |
| MT326159.1 | 100 | 100 | 100 | Severe acute respiratory syndrome coronavirus 2 |
| | | | | isolate SARS-CoV-2/human/USA/WA-UW- |
| | | | | 1722/2020, complete genome |
| MT326157.1 | 100 | 100 | 100 | Severe acute respiratory syndrome coronavirus 2 |
| | | | | isolate SARS-CoV-2/human/USA/WA-UW- |
| | | | | 1708/2020 ORF1ab polyprotein (ORF1ab) and |
| | | | | ORF1a polyprotein (ORF1ab) genes, partial cds; |
| | | | | and surface glycoprotein (S), ORF3a protein |
| | | | | (ORF3a), envelope protein (E), membrane |
| | | | | glycoprotein (M), ORF6 protein (ORF6), ORF7a |
| | | | | protein (ORF7a), ORF7b (ORF7b), ORF8 |
| | | | | protein (ORF8), nucleocapsid phosphoprotein |
| | | | | (N), and ORF10 protein (ORF10) genes, |
| | | | | complete cds |
| MT326109.1 | 100 | 100 | 100 | Severe acute respiratory syndrome coronavirus 2 |
| | | | | isolate SARS-CoV-2/human/USA/WA-UW- |
| | | | | 1826/2020 ORF1ab polyprotein (ORF1ab), |
| | | | | ORF1a polyprotein (ORF1ab), surface |
| | | | | glycoprotein (S), ORF3a protein (ORF3a), |
| | | | | envelope protein (E), membrane glycoprotein |
| | | | | (M), ORF6 protein (ORF6), ORF7a protein |
| | | | | (ORF7a), ORF7b (ORF7b), ORF8 protein |
| | | | | (ORF8), nucleocapsid phosphoprotein (N), and |
| | | | | ORF10 protein (ORF10) genes, complete cds |
| MT326151.1 | 100 | 100 | 100 | Severe acute respiratory syndrome coronavirus 2 |
| | | | | isolate SARS-CoV-2/human/USA/WA-UW- |
| | | | | 1705/2020, complete genome |
| MT326107.1 | 100 | 100 | 100 | Severe acute respiratory syndrome coronavirus 2 |
| | | | | isolate SARS-CoV-2/human/USA/WA-UW- |
| | | | | 1827/2020 ORF1ab polyprotein (ORF1ab), |
| | | | | ORF1a polyprotein (ORF1ab), surface |
| | | | | glycoprotein (S), ORF3a protein (ORF3a), |
| | | | | envelope protein (E), membrane glycoprotein |
| | | | | (M), ORF6 protein (ORF6), ORF7a protein |
| | | | | (ORF7a), ORF7b (ORF7b), ORF8 protein |
| | | | | (ORF8), nucleocapsid phosphoprotein (N), and |
| | | | | ORF10 protein (ORF10) genes, complete cds |
| MT326103.1 | 100 | 100 | 100 | Severe acute respiratory syndrome coronavirus 2 |
| | | | | isolate SARS-CoV-2/human/USA/UNKNOWN- |
| | | | | UW-1817/2020, complete genome |
| MT326142.1 | 100 | 100 | 100 | Severe acute respiratory syndrome coronavirus 2 |
| | | | | isolate SARS-CoV-2/human/USA/WA-UW- |
| | | | | 1684/2020 ORF1ab polyprotein (ORF1ab) and |
| | | | | ORF1a polyprotein (ORF1ab) genes, partial cds; |
| | | | | surface glycoprotein (S) and ORF3a protein |
| | | | | (ORF3a) genes, complete cds; envelope protein |
| | | | | (E), membrane glycoprotein (M), ORF6 protein |
| | | | | (ORF6), and ORF7a protein (ORF7a) genes, |
| | | | | partial cds; and ORF7b (ORF7b), ORF8 protein |
| | | | | (ORF8), nucleocapsid phosphoprotein (N), and |
| | | | | ORF10 protein (ORF10) genes, complete cds |
| MT326141.1 | 100 | 100 | 100 | Severe acute respiratory syndrome coronavirus 2 |
| | | | | isolate SARS-CoV-2/human/USA/WA-UW- |
| | | | | 1690/2020 ORF1ab polyprotein (ORF1ab) and |
| | | | | ORF1a polyprotein (ORF1ab) genes, partial cds; |
| | | | | and surface glycoprotein (S), ORF3a protein |
| | | | | (ORF3a), envelope protein (E), membrane |
| | | | | glycoprotein (M), ORF6 protein (ORF6), ORF7a |
| | | | | protein (ORF7a), ORF7b (ORF7b), ORF8 |
| | | | | protein (ORF8), nucleocapsid phosphoprotein |
| | | | | (N), and ORF10 protein (ORF10) genes, |
| | | | | complete cds |
| MT326093.1 | 100 | 100 | 100 | Severe acute respiratory syndrome coronavirus 2 |
| | | | | isolate SARS-CoV-2/human/USA/WA-UW- |
| | | | | 1775/2020, complete genome |
| MT326090.1 | 100 | 100 | 100 | Severe acute respiratory syndrome coronavirus 2 |
| | | | | isolate SARS-CoV-2/human/USA/WA-UW- |
| | | | | 1796/2020, complete genome |
| MT325574.1 | 100 | 100 | 100 | Severe acute respiratory syndrome coronavirus 2 |
| | | | | isolate SARS-CoV- |
| | | | | 2/human/USA/GA2185/2020, complete |
| | | | | genome |
| MT324684.1 | 100 | 100 | 100 | Severe acute respiratory syndrome coronavirus 2 |
| | | | | isolate SARS-CoV-2/ENV/USA/UF-3/2020, |
| | | | | complete genome |
| MT326155.1 | 100 | 100 | 100 | Severe acute respiratory syndrome coronavirus 2 |
| | | | | isolate SARS-CoV-2/human/USA/WA-UW- |
| | | | | 1709/2020 ORF1ab polyprotein (ORF1ab), |
| | | | | ORF1a polyprotein (ORF1ab), surface |
| | | | | glycoprotein (S), ORF3a protein (ORF3a), |
| | | | | envelope protein (E), membrane glycoprotein |
| | | | | (M), ORF6 protein (ORF6), ORF7a protein |
| | | | | (ORF7a), ORF7b (ORF7b), ORF8 protein |
| | | | | (ORF8), nucleocapsid phosphoprotein (N), and |
| | | | | ORF10 protein (ORF10) genes, complete cds |
| MT326144.1 | 100 | 100 | 100 | Severe acute respiratory syndrome coronavirus 2 |
| | | | | isolate SARS-CoV-2/human/USA/WA-UW- |
| | | | | 1695/2020 ORF1ab polyprotein (ORF1ab), |
| | | | | ORF1a polyprotein (ORF1ab), surface |
| | | | | glycoprotein (S), ORF3a protein (ORF3a), |
| | | | | envelope protein (E), membrane glycoprotein |
| | | | | (M), ORF6 protein (ORF6), ORF7a protein |
| | | | | (ORF7a), ORF7b (ORF7b), ORF8 protein |
| | | | | (ORF8), nucleocapsid phosphoprotein (N), and |
| | | | | ORF10 protein (ORF10) genes, complete cds |
| MT326181.1 | 100 | 100 | 100 | Severe acute respiratory syndrome coronavirus 2 |
| | | | | isolate SARS-CoV-2/human/USA/IL-UW- |
| | | | | 1311/2020, complete genome |
| MT326179.1 | 100 | 100 | 100 | Severe acute respiratory syndrome coronavirus 2 |
| | | | | isolate SARS-CoV-2/human/USA/WA-UW- |
| | | | | 1572/2020 ORF1ab polyprotein (ORF1ab), |
| | | | | ORF1a polyprotein (ORF1ab), surface |
| | | | | glycoprotein (S), ORF3a protein (ORF3a), |
| | | | | envelope protein (E), membrane glycoprotein |
| | | | | (M), ORF6 protein (ORF6), ORF7a protein |
| | | | | (ORF7a), ORF7b (ORF7b), ORF8 protein |
| | | | | (ORF8), nucleocapsid phosphoprotein (N), and |
| | | | | ORF10 protein (ORF10) genes, complete cds |
| MT326130.1 | 100 | 100 | 100 | Severe acute respiratory syndrome coronavirus 2 |
| | | | | isolate SARS-CoV-2/human/USA/WA-UW- |
| | | | | 1697/2020, complete genome |
| MT326175.1 | 100 | 100 | 100 | Severe acute respiratory syndrome coronavirus 2 |
| | | | | isolate SARS-CoV-2/human/USA/WA-UW- |
| | | | | 1616/2020 ORF1ab polyprotein (ORF1ab), |
| | | | | ORF1a polyprotein (ORF1ab), surface |
| | | | | glycoprotein (S), ORF3a protein (ORF3a), |
| | | | | envelope protein (E), membrane glycoprotein |
| | | | | (M), ORF6 protein (ORF6), ORF7a protein |
| | | | | (ORF7a), ORF7b (ORF7b), ORF8 protein |
| | | | | (ORF8), nucleocapsid phosphoprotein (N), and |
| | | | | ORF10 protein (ORF10) genes, complete cds |
| MT326126.1 | 100 | 100 | 100 | Severe acute respiratory syndrome coronavirus 2 |
| | | | | isolate SARS-CoV-2/human/USA/WA-UW- |
| | | | | 1659/2020 ORF1ab polyprotein (ORF1ab) and |
| | | | | ORF1a polyprotein, nspll region, (ORF1ab) |
| | | | | genes, partial cds; and surface glycoprotein (S), |
| | | | | ORF3a protein (ORF3a), envelope protein (E), |
| | | | | membrane glycoprotein (M), ORF6 protein |
| | | | | (ORF6), ORF7a protein (ORF7a), ORF7b |
| | | | | (ORF7b), ORF8 protein (ORF8), nucleocapsid |
| | | | | phosphoprotein (N), and ORF10 protein |
| | | | | (ORF10) genes, complete cds |
| MT326170.1 | 100 | 100 | 100 | Severe acute respiratory syndrome coronavirus 2 |
| | | | | isolate SARS-CoV-2/human/USA/WA-UW- |
| | | | | 1762/2020 ORF1ab polyprotein (ORF1ab), |
| | | | | ORF1a polyprotein (ORF1ab), surface |
| | | | | glycoprotein (S), ORF3a protein (ORF3a), |
| | | | | envelope protein (E), membrane glycoprotein |
| | | | | (M), ORF6 protein (ORF6), ORF7a protein |
| | | | | (ORF7a), ORF7b (ORF7b), ORF8 protein |
| | | | | (ORF8), nucleocapsid phosphoprotein (N), and |
| | | | | ORF10 protein (ORF10) genes, complete cds |
| MT326124.1 | 100 | 100 | 100 | Severe acute respiratory syndrome coronavirus 2 |
| | | | | isolate SARS-CoV-2/human/USA/WA-UW- |
| | | | | 1672/2020, complete genome |
| MT326166.1 | 100 | 100 | 100 | Severe acute respiratory syndrome coronavirus 2 |
| | | | | isolate SARS-CoV-2/human/USA/WA-UW- |
| | | | | 1735/2020, complete genome |
| MT326119.1 | 100 | 100 | 100 | Severe acute respiratory syndrome coronavirus 2 |
| | | | | isolate SARS-CoV-2/human/USA/WA-UW- |
| | | | | 1661/2020, complete genome |
| MT326164.1 | 100 | 100 | 100 | Severe acute respiratory syndrome coronavirus 2 |
| | | | | isolate SARS-CoV-2/human/USA/WA-UW- |
| | | | | 1750/2020, complete genome |
| MT326117.1 | 100 | 100 | 100 | Severe acute respiratory syndrome coronavirus 2 |
| | | | | isolate SARS-CoV-2/human/USA/WA-UW- |
| | | | | 1688/2020, complete genome |
| MT326116.1 | 100 | 100 | 100 | Severe acute respiratory syndrome coronavirus 2 |
| | | | | isolate SARS-CoV-2/human/USA/WA-UW- |
| | | | | 1683/2020, complete genome |
| MT326115.1 | 100 | 100 | 100 | Severe acute respiratory syndrome coronavirus 2 |
| | | | | isolate SARS-CoV-2/human/USA/WA-UW- |
| | | | | 1675/2020 ORF1ab polyprotein (ORF1ab) and |
| | | | | ORF1a polyprotein (ORF1ab) genes, partial cds; |
| | | | | surface glycoprotein (S) and ORF3a protein |
| | | | | (ORF3a) genes, complete cds; envelope protein |
| | | | | (E) gene, partial cds; and membrane |
| | | | | glycoprotein (M), ORF6 protein (ORF6), ORF7a |
| | | | | protein (ORF7a), ORF7b (ORF7b), ORF8 |
| | | | | protein (ORF8), nucleocapsid phosphoprotein |
| | | | | (N), and ORF10 protein (ORF10) genes, |
| | | | | complete cds |
| MT326113.1 | 100 | 100 | 100 | Severe acute respiratory syndrome coronavirus 2 |
| | | | | isolate SARS-CoV-2/human/USA/WA-UW- |
| | | | | 1646/2020, complete genome |
| MT326112.1 | 100 | 100 | 100 | Severe acute respiratory syndrome coronavirus 2 |
| | | | | isolate SARS-CoV-2/human/USA/WA-UW- |
| | | | | 1644/2020, complete genome |
| MT326111.1 | 100 | 100 | 100 | Severe acute respiratory syndrome coronavirus 2 |
| | | | | isolate SARS-CoV-2/human/USA/WA-UW- |
| | | | | 1822/2020, complete genome |
| MT326110.1 | 100 | 100 | 100 | Severe acute respiratory syndrome coronavirus 2 |
| | | | | isolate SARS-CoV-2/human/USA/WA-UW- |
| | | | | 1821/2020 ORF1ab polyprotein (ORF1ab), |
| | | | | ORF1a polyprotein (ORF1ab), surface |
| | | | | glycoprotein (S), ORF3a protein (ORF3a), |
| | | | | envelope protein (E), membrane glycoprotein |
| | | | | (M), ORF6 protein (ORF6), ORF7a protein |
| | | | | (ORF7a), ORF7b (ORF7b), ORF8 protein |
| | | | | (ORF8), nucleocapsid phosphoprotein (N), and |
| | | | | ORF10 protein (ORF10) genes, complete cds |
| MT326152.1 | 100 | 100 | 100 | Severe acute respiratory syndrome coronavirus 2 |
| | | | | isolate SARS-CoV-2/human/USA/WA-UW- |
| | | | | 1706/2020, complete genome |
| MT326108.1 | 100 | 100 | 100 | Severe acute respiratory syndrome coronavirus 2 |
| | | | | isolate SARS-CoV-2/human/USA/WA-UW- |
| | | | | 1825/2020 ORF1ab polyprotein (ORF1ab) and |
| | | | | ORF1a polyprotein, nspll region, (ORF1ab) |
| | | | | genes, partial cds; and surface glycoprotein (S), |
| | | | | ORF3a protein (ORF3a), envelope protein (E), |
| | | | | membrane glycoprotein (M), ORF6 protein |
| | | | | (ORF6), ORF7a protein (ORF7a), ORF7b |
| | | | | (ORF7b), ORF8 protein (ORF8), nucleocapsid |
| | | | | phosphoprotein (N), and ORF10 protein |
| | | | | (ORF10) genes, complete cds |
| MT326150.1 | 100 | 100 | 100 | Severe acute respiratory syndrome coronavirus 2 |
| | | | | isolate SARS-CoV-2/human/USA/WA-UW- |
| | | | | 1707/2020, complete genome |
| MT326149.1 | 100 | 100 | 100 | Severe acute respiratory syndrome coronavirus 2 |
| | | | | isolate SARS-CoV-2/human/USA/WA-UW- |
| | | | | 1745/2020, complete genome |
| MT326102.1 | 100 | 100 | 100 | Severe acute respiratory syndrome coronavirus 2 |
| | | | | isolate SARS-CoV-2/human/USA/UNKNOWN- |
| | | | | UW-1818/2020 ORF1ab polyprotein (ORF1ab), |
| | | | | ORF1a polyprotein (ORF1ab), surface |
| | | | | glycoprotein (S), ORF3a protein (ORF3a), |
| | | | | envelope protein (E), membrane glycoprotein |
| | | | | (M), ORF6 protein (ORF6), ORF7a protein |
| | | | | (ORF7a), ORF7b (ORF7b), ORF8 protein |
| | | | | (ORF8), nucleocapsid phosphoprotein (N), and |
| | | | | ORF10 protein (ORF10) genes, complete cds |
| MT326143.1 | 100 | 100 | 100 | Severe acute respiratory syndrome coronavirus 2 |
| | | | | isolate SARS-CoV-2/human/USA/WA-UW- |
| | | | | 1682/2020 ORF1ab polyprotein (ORF1ab) and |
| | | | | ORF1a polyprotein (ORF1ab) genes, partial cds; |
| | | | | and surface glycoprotein (S), ORF3a protein |
| | | | | (ORF3a), envelope protein (E), membrane |
| | | | | glycoprotein (M), ORF6 protein (ORF6), ORF7a |
| | | | | protein (ORF7a), ORF7b (ORF7b), ORF8 |
| | | | | protein (ORF8), nucleocapsid phosphoprotein |
| | | | | (N), and ORF10 protein (ORF10) genes, |
| | | | | complete cds |
| MT326100.1 | 100 | 100 | 100 | Severe acute respiratory syndrome coronavirus 2 |
| | | | | isolate SARS-CoV-2/human/USA/WA-UW- |
| | | | | 1805/2020, complete genome |
| MT326133.1 | 100 | 100 | 100 | Severe acute respiratory syndrome coronavirus 2 |
| | | | | isolate SARS-CoV-2/human/USA/WA-UW- |
| | | | | 1712/2020, complete genome |
| MT326092.1 | 100 | 100 | 100 | Severe acute respiratory syndrome coronavirus 2 |
| | | | | isolate SARS-CoV-2/human/USA/WA-UW- |
| | | | | 1774/2020, complete genome |
| MT326123.1 | 100 | 100 | 100 | Severe acute respiratory syndrome coronavirus 2 |
| | | | | isolate SARS-CoV-2/human/USA/WA-UW- |
| | | | | 1656/2020, complete genome |
| MT326089.1 | 100 | 100 | 100 | Severe acute respiratory syndrome coronavirus 2 |
| | | | | isolate SARS-CoV-2/human/USA/WA-UW- |
| | | | | 1792/2020, complete genome |
| MT325573.1 | 100 | 100 | 100 | Severe acute respiratory syndrome coronavirus 2 |
| | | | | isolate SARS-CoV- |
| | | | | 2/human/USA/GA2120/2020, complete |
| | | | | genome |
| MT334573.1 | 100 | 100 | 100 | Severe acute respiratory syndrome coronavirus 2 |
| | | | | isolate SARS-CoV-2/human/USA/UT- |
| | | | | 00361/2020 ORF1ab polyprotein (ORF1ab), |
| | | | | ORF1a polyprotein (ORF1ab), surface |
| | | | | glycoprotein (S), ORF3a protein (ORF3a), |
| | | | | envelope protein (E), membrane glycoprotein |
| | | | | (M), ORF6 protein (ORF6), ORF7a protein |
| | | | | (ORF7a), ORF7b (ORF7b), ORF8 protein |
| | | | | (ORF8), nucleocapsid phosphoprotein (N), and |
| | | | | ORF10 protein (ORF10) genes, complete cds |
| MT334572.1 | 100 | 100 | 100 | Severe acute respiratory syndrome coronavirus 2 |
| | | | | isolate SARS-CoV-2/human/USA/UT- |
| | | | | 00352/2020 ORF1ab polyprotein (ORF1ab), |
| | | | | ORF1a polyprotein (ORF1ab), surface |
| | | | | glycoprotein (S), ORF3a protein (ORF3a), |
| | | | | envelope protein (E), membrane glycoprotein |
| | | | | (M), ORF6 protein (ORF6), ORF7a protein |
| | | | | (ORF7a), ORF7b (ORF7b), ORF8 protein |
| | | | | (ORF8), nucleocapsid phosphoprotein (N), and |
| | | | | ORF10 protein (ORF10) genes, complete cds |
| MT334571.1 | 100 | 100 | 100 | Severe acute respiratory syndrome coronavirus 2 |
| | | | | isolate SARS-CoV-2/human/USA/UT- |
| | | | | 00350/2020 ORF1ab polyprotein (ORF1ab), |
| | | | | ORF1a polyprotein (ORF1ab), surface |
| | | | | glycoprotein (S), ORF3a protein (ORF3a), |
| | | | | envelope protein (E), membrane glycoprotein |
| | | | | (M), and ORF6 protein (ORF6) genes, complete |
| | | | | cds; ORF7a protein (ORF7a) and ORF7b |
| | | | | (ORF7b) genes, partial cds; and ORF8 protein |
| | | | | (ORF8), nucleocapsid phosphoprotein (N), and |
| | | | | ORF10 protein (ORF10) genes, complete cds |
| MT334570.1 | 100 | 100 | 100 | Severe acute respiratory syndrome coronavirus 2 |
| | | | | isolate SARS-CoV-2/human/USA/UT- |
| | | | | 00349/2020 ORF1ab polyprotein (ORF1ab), |
| | | | | ORF1a polyprotein (ORF1ab), surface |
| | | | | glycoprotein (S), ORF3a protein (ORF3a), |
| | | | | envelope protein (E), membrane glycoprotein |
| | | | | (M), ORF6 protein (ORF6), ORF7a protein |
| | | | | (ORF7a), ORF7b (ORF7b), ORF8 protein |
| | | | | (ORF8), nucleocapsid phosphoprotein (N), and |
| | | | | ORF10 protein (ORF10) genes, complete cds |
| MT334569.1 | 100 | 100 | 100 | Severe acute respiratory syndrome coronavirus 2 |
| | | | | isolate SARS-CoV-2/human/USA/UT- |
| | | | | 00348/2020 ORF1ab polyprotein (ORF1ab), |
| | | | | ORF1a polyprotein (ORF1ab), surface |
| | | | | glycoprotein (S), ORF3a protein (ORF3a), |
| | | | | envelope protein (E), membrane glycoprotein |
| | | | | (M), ORF6 protein (ORF6), ORF7a protein |
| | | | | (ORF7a), ORF7b (ORF7b), ORF8 protein |
| | | | | (ORF8), nucleocapsid phosphoprotein (N), and |
| | | | | ORF10 protein (ORF10) genes, complete cds |
| MT334568.1 | 100 | 100 | 100 | Severe acute respiratory syndrome coronavirus 2 |
| | | | | isolate SARS-CoV-2/human/USA/UT- |
| | | | | 00347/2020 ORF1ab polyprotein (ORF1ab), |
| | | | | ORF1a polyprotein (ORF1ab), surface |
| | | | | glycoprotein (S), ORF3a protein (ORF3a), |
| | | | | envelope protein (E), membrane glycoprotein |
| | | | | (M), and ORF6 protein (ORF6) genes, complete |
| | | | | cds; ORF7a protein (ORF7a) and ORF7b |
| | | | | (ORF7b) genes, partial cds; and ORF8 protein |
| | | | | (ORF8), nucleocapsid phosphoprotein (N), and |
| | | | | ORF10 protein (ORF10) genes, complete cds |
| MT334567.1 | 100 | 100 | 100 | Severe acute respiratory syndrome coronavirus 2 |
| | | | | isolate SARS-CoV-2/human/USA/UT- |
| | | | | 00346/2020 ORF1ab polyprotein (ORF1ab), |
| | | | | ORF1a polyprotein (ORF1ab), surface |
| | | | | glycoprotein (S), ORF3a protein (ORF3a), |
| | | | | envelope protein (E), membrane glycoprotein |
| | | | | (M), and ORF6 protein (ORF6) genes, complete |
| | | | | cds; ORF7a protein (ORF7a) and ORF7b |
| | | | | (ORF7b) genes, partial cds; and ORF8 protein |
| | | | | (ORF8), nucleocapsid phosphoprotein (N), and |
| | | | | ORF10 protein (ORF10) genes, complete cds |
| MT334566.1 | 100 | 100 | 100 | Severe acute respiratory syndrome coronavirus 2 |
| | | | | isolate SARS-CoV-2/human/USA/UT- |
| | | | | 00345/2020 ORF1ab polyprotein (ORF1ab), |
| | | | | ORF1a polyprotein (ORF1ab), surface |
| | | | | glycoprotein (S), ORF3a protein (ORF3a), |
| | | | | envelope protein (E), membrane glycoprotein |
| | | | | (M), and ORF6 protein (ORF6) genes, complete |
| | | | | cds; ORF7a protein (ORF7a) and ORF7b |
| | | | | (ORF7b) genes, partial cds; and ORF8 protein |
| | | | | (ORF8), nucleocapsid phosphoprotein (N), and |
| | | | | ORF10 protein (ORF10) genes, complete cds |
| MT334565.1 | 100 | 100 | 100 | Severe acute respiratory syndrome coronavirus 2 |
| | | | | isolate SARS-CoV-2/human/USA/UT- |
| | | | | 00344/2020 ORF1ab polyprotein (ORF1ab), |
| | | | | ORF1a polyprotein (ORF1ab), surface |
| | | | | glycoprotein (S), ORF3a protein (ORF3a), |
| | | | | envelope protein (E), membrane glycoprotein |
| | | | | (M), and ORF6 protein (ORF6) genes, complete |
| | | | | cds; ORF7a protein (ORF7a) and ORF7b |
| | | | | (ORF7b) genes, partial cds; and ORF8 protein |
| | | | | (ORF8), nucleocapsid phosphoprotein (N), and |
| | | | | ORF10 protein (ORF10) genes, complete cds |
| MT334564.1 | 100 | 100 | 100 | Severe acute respiratory syndrome coronavirus 2 |
| | | | | isolate SARS-CoV-2/human/USA/UT- |
| | | | | 00342/2020 ORF1ab polyprotein (ORF1ab), |
| | | | | ORF1a polyprotein (ORF1ab), surface |
| | | | | glycoprotein (S), ORF3a protein (ORF3a), |
| | | | | envelope protein (E), membrane glycoprotein |
| | | | | (M), and ORF6 protein (ORF6) genes, complete |
| | | | | cds; ORF7a protein (ORF7a) and ORF7b |
| | | | | (ORF7b) genes, partial cds; and ORF8 protein |
| | | | | (ORF8), nucleocapsid phosphoprotein (N), and |
| | | | | ORF10 protein (ORF10) genes, complete cds |
| MT334563.1 | 100 | 100 | 100 | Severe acute respiratory syndrome coronavirus 2 |
| | | | | isolate SARS-CoV-2/human/USA/UT- |
| | | | | 00303/2020 ORF1ab polyprotein (ORF1ab), |
| | | | | ORF1a polyprotein (ORF1ab), surface |
| | | | | glycoprotein (S), ORF3a protein (ORF3a), |
| | | | | envelope protein (E), membrane glycoprotein |
| | | | | (M), ORF6 protein (ORF6), ORF7a protein |
| | | | | (ORF7a), ORF7b (ORF7b), ORF8 protein |
| | | | | (ORF8), nucleocapsid phosphoprotein (N), and |
| | | | | ORF10 protein (ORF10) genes, complete cds |
| MT334561.1 | 100 | 100 | 100 | Severe acute respiratory syndrome coronavirus 2 |
| | | | | isolate SARS-CoV-2/human/USA/UT- |
| | | | | 00301/2020, complete genome |
| MT334560.1 | 100 | 100 | 100 | Severe acute respiratory syndrome coronavirus 2 |
| | | | | isolate SARS-CoV-2/human/USA/UT- |
| | | | | 00300/2020 ORF1ab polyprotein (ORF1ab), |
| | | | | ORF1a polyprotein (ORF1ab), surface |
| | | | | glycoprotein (S), ORF3a protein (ORF3a), |
| | | | | envelope protein (E), membrane glycoprotein |
| | | | | (M), and ORF6 protein (ORF6) genes, complete |
| | | | | cds; ORF7a gene, complete sequence; and |
| | | | | ORF7b (ORF7b), ORF8protein (ORF8), |
| | | | | nucleocapsid phosphoprotein (N), and ORF10 |
| | | | | protein (ORF10) genes, complete cds |
| MT334559.1 | 100 | 100 | 100 | Severe acute respiratory syndrome coronavirus 2 |
| | | | | isolate SARS-CoV-2/human/USA/UT- |
| | | | | 00297/2020 ORF1ab polyprotein (ORF1ab), |
| | | | | ORF1a polyprotein (ORF1ab), surface |
| | | | | glycoprotein (S), ORF3a protein (ORF3a), |
| | | | | envelope protein (E), membrane glycoprotein |
| | | | | (M), ORF6 protein (ORF6), ORF7a protein |
| | | | | (ORF7a), ORF7b (ORF7b), ORF8 protein |
| | | | | (ORF8), nucleocapsid phosphoprotein (N), and |
| | | | | ORF10 protein (ORF10) genes, complete cds |
| MT334558.1 | 100 | 100 | 100 | Severe acute respiratory syndrome coronavirus 2 |
| | | | | isolate SARS-CoV-2/human/USA/UT- |
| | | | | 00291/2020 ORF1ab polyprotein (ORF1ab), |
| | | | | ORF1a polyprotein (ORF1ab), surface |
| | | | | glycoprotein (S), ORF3a protein (ORF3a), |
| | | | | envelope protein (E), membrane glycoprotein |
| | | | | (M), and ORF6 protein (ORF6) genes, complete |
| | | | | cds; ORF7a protein (ORF7a) and ORF7b |
| | | | | (ORF7b) genes, partial cds; and ORF8 protein |
| | | | | (ORF8), nucleocapsid phosphoprotein (N), and |
| | | | | ORF10 protein (ORF10) genes, complete cds |
| MT334557.1 | 100 | 100 | 100 | Severe acute respiratory syndrome coronavirus 2 |
| | | | | isolate SARS-CoV-2/human/USA/UT- |
| | | | | 00290/2020, complete genome |
| MT334556.1 | 100 | 100 | 100 | Severe acute respiratory syndrome coronavirus 2 |
| | | | | isolate SARS-CoV-2/human/USA/UT- |
| | | | | 00289/2020 ORF1ab polyprotein (ORF1ab), |
| | | | | ORF1a polyprotein (ORF1ab), surface |
| | | | | glycoprotein (S), ORF3a protein (ORF3a), |
| | | | | envelope protein (E), membrane glycoprotein |
| | | | | (M), ORF6 protein (ORF6), ORF7a protein |
| | | | | (ORF7a), ORF7b (ORF7b), ORF8 protein |
| | | | | (ORF8), nucleocapsid phosphoprotein (N), and |
| | | | | ORF10 protein (ORF10) genes, complete cds |
| MT334554.1 | 100 | 100 | 100 | Severe acute respiratory syndrome coronavirus 2 |
| | | | | isolate SARS-CoV-2/human/USA/UT- |
| | | | | 00287/2020 ORF1ab polyprotein (ORF1ab), |
| | | | | ORF1a polyprotein (ORF1ab), surface |
| | | | | glycoprotein (S), ORF3a protein (ORF3a), |
| | | | | envelope protein (E), membrane glycoprotein |
| | | | | (M), ORF6 protein (ORF6), ORF7a protein |
| | | | | (ORF7a), ORF7b (ORF7b), ORF8 protein |
| | | | | (ORF8), nucleocapsid phosphoprotein (N), and |
| | | | | ORF10 protein (ORF10) genes, complete cds |
| MT334553.1 | 100 | 100 | 100 | Severe acute respiratory syndrome coronavirus 2 |
| | | | | isolate SARS-CoV-2/human/USA/UT- |
| | | | | 00284/2020 ORF1ab polyprotein (ORF1ab), |
| | | | | ORF1a polyprotein (ORF1ab), surface |
| | | | | glycoprotein (S), ORF3a protein (ORF3a), |
| | | | | envelope protein (E), membrane glycoprotein |
| | | | | (M), and ORF6 protein (ORF6) genes, complete |
| | | | | cds; ORF7a protein (ORF7a) and ORF7b |
| | | | | (ORF7b) genes, partial cds; and ORF8 protein |
| | | | | (ORF8), nucleocapsid phosphoprotein (N), and |
| | | | | ORF10 protein (ORF10) genes, complete cds |
| MT334552.1 | 100 | 100 | 100 | Severe acute respiratory syndrome coronavirus 2 |
| | | | | isolate SARS-CoV-2/human/USA/UT- |
| | | | | 00159/2020 ORF1ab polyprotein (ORF1ab), |
| | | | | ORF1a polyprotein (ORF1ab), surface |
| | | | | glycoprotein (S), ORF3a protein (ORF3a), |
| | | | | envelope protein (E), membrane glycoprotein |
| | | | | (M), ORF6 protein (ORF6), ORF7a protein |
| | | | | (ORF7a), ORF7b (ORF7b), ORF8 protein |
| | | | | (ORF8), nucleocapsid phosphoprotein (N), and |
| | | | | ORF10 protein (ORF10) genes, complete cds |
| MT334551.1 | 100 | 100 | 100 | Severe acute respiratory syndrome coronavirus 2 |
| | | | | isolate SARS-CoV-2/human/USA/UT- |
| | | | | 00090/2020 ORF1ab polyprotein (ORF1ab), |
| | | | | ORF1a polyprotein (ORF1ab), surface |
| | | | | glycoprotein (S), ORF3a protein (ORF3a), |
| | | | | envelope protein (E), membrane glycoprotein |
| | | | | (M), and ORF6 protein (ORF6) genes, complete |
| | | | | cds; ORF7a protein (ORF7a) and ORF7b |
| | | | | (ORF7b) genes, partial cds; and ORF8 protein |
| | | | | (ORF8), nucleocapsid phosphoprotein (N), and |
| | | | | ORF10 protein (ORF10) genes, complete cds |
| MT334550.1 | 100 | 100 | 100 | Severe acute respiratory syndrome coronavirus 2 |
| | | | | isolate SARS-CoV-2/human/USA/UT- |
| | | | | 00089/2020 ORF1ab polyprotein (ORF1ab), |
| | | | | ORF1a polyprotein (ORF1ab), surface |
| | | | | glycoprotein (S), ORF3a protein (ORF3a), |
| | | | | envelope protein (E), membrane glycoprotein |
| | | | | (M), ORF6 protein (ORF6), ORF7a protein |
| | | | | (ORF7a), ORF7b (ORF7b), ORF8 protein |
| | | | | (ORF8), nucleocapsid phosphoprotein (N), and |
| | | | | ORF10 protein (ORF10) genes, complete cds |
| MT334548.1 | 100 | 100 | 100 | Severe acute respiratory syndrome coronavirus 2 |
| | | | | isolate SARS-CoV-2/human/USA/UT- |
| | | | | 00034/2020 ORF1ab polyprotein (ORF1ab), |
| | | | | ORF1a polyprotein (ORF1ab), surface |
| | | | | glycoprotein (S), ORF3a protein (ORF3a), |
| | | | | envelope protein (E), membrane glycoprotein |
| | | | | (M), ORF6 protein (ORF6), ORF7a protein |
| | | | | (ORF7a), ORF7b (ORF7b), ORF8 protein |
| | | | | (ORF8), nucleocapsid phosphoprotein (N), and |
| | | | | ORF10 protein (ORF10) genes, complete cds |
| MT334547.1 | 100 | 100 | 100 | Severe acute respiratory syndrome coronavirus 2 |
| | | | | isolate SARS-CoV-2/human/USA/UT- |
| | | | | 00033/2020, complete genome |
| MT334555.1 | 100 | 100 | 100 | Severe acute respiratory syndrome coronavirus 2 |
| | | | | isolate SARS-CoV-2/human/USA/UT- |
| | | | | 00288/2020 ORF1ab polyprotein (ORF1ab), |
| | | | | ORF1a polyprotein (ORF1ab), surface |
| | | | | glycoprotein (S), ORF3a protein (ORF3a), |
| | | | | envelope protein (E), membrane glycoprotein |
| | | | | (M), and ORF6 protein (ORF6) genes, complete |
| | | | | cds; ORF7a protein (ORF7a) and ORF7b |
| | | | | (ORF7b) genes, partial cds; and ORF8 protein |
| | | | | (ORF8), nucleocapsid phosphoprotein (N), and |
| | | | | ORF10 protein (ORF10) genes, complete cds |
| MT334546.1 | 100 | 100 | 100 | Severe acute respiratory syndrome coronavirus 2 |
| | | | | isolate SARS-CoV-2/human/USA/UT- |
| | | | | 00032/2020, complete genome |
| MT334545.1 | 100 | 100 | 100 | Severe acute respiratory syndrome coronavirus 2 |
| | | | | isolate SARS-CoV-2/human/USA/UT- |
| | | | | 00031/2020 ORF1ab polyprotein (ORF1ab), |
| | | | | ORF1a polyprotein (ORF1ab), surface |
| | | | | glycoprotein (S), ORF3a protein (ORF3a), |
| | | | | envelope protein (E), membrane glycoprotein |
| | | | | (M), ORF6 protein (ORF6), ORF7a protein |
| | | | | (ORF7a), ORF7b (ORF7b), ORF8 protein |
| | | | | (ORF8), nucleocapsid phosphoprotein (N), and |
| | | | | ORF10 protein (ORF10) genes, complete cds |
| MT334544.1 | 100 | 100 | 100 | Severe acute respiratory syndrome coronavirus 2 |
| | | | | isolate SARS-CoV-2/human/USA/UT- |
| | | | | 00028/2020, complete genome |
| MT334543.1 | 100 | 100 | 100 | Severe acute respiratory syndrome coronavirus 2 |
| | | | | isolate SARS-CoV-2/human/USA/UT- |
| | | | | 00027/2020ORF1ab polyprotein (ORF1ab), |
| | | | | ORF1a polyprotein (ORF1ab), surface |
| | | | | glycoprotein (S), ORF3a protein (ORF3a), |
| | | | | envelope protein (E), membrane glycoprotein |
| | | | | (M), ORF6 protein (ORF6), ORF7a protein |
| | | | | (ORF7a), ORF7b (ORF7b), ORF8 protein |
| | | | | (ORF8), nucleocapsid phosphoprotein (N), and |
| | | | | ORF10 protein (ORF10) genes, complete cds |
| MT334542.1 | 100 | 100 | 100 | Severe acute respiratory syndrome coronavirus 2 |
| | | | | isolate SARS-CoV-2/human/USA/UT- |
| | | | | 00025/2020, complete genome |
| MT334541.1 | 100 | 100 | 100 | Severe acute respiratory syndrome coronavirus 2 |
| | | | | isolate SARS-CoV-2/human/USA/UT- |
| | | | | 00023/2020, complete genome |
| MT334540.1 | 100 | 100 | 100 | Severe acute respiratory syndrome coronavirus 2 |
| | | | | isolate SARS-CoV-2/human/USA/UT- |
| | | | | 00022/2020, complete genome |
| MT334539.1 | 100 | 100 | 100 | Severe acute respiratory syndrome coronavirus 2 |
| | | | | isolate SARS-CoV-2/human/USA/UT- |
| | | | | 00021/2020, complete genome |
| MT334538.1 | 100 | 100 | 100 | Severe acute respiratory syndrome coronavirus 2 |
| | | | | isolate SARS-CoV-2/human/USA/UT- |
| | | | | 00020/2020, complete genome |
| MT334537.1 | 100 | 100 | 100 | Severe acute respiratory syndrome coronavirus 2 |
| | | | | isolate SARS-CoV-2/human/USA/UT- |
| | | | | 00019/2020, complete genome |
| MT334536.1 | 100 | 100 | 100 | Severe acute respiratory syndrome coronavirus 2 |
| | | | | isolate SARS-CoV-2/human/USA/UT- |
| | | | | 00017/2020, complete genome |
| MT334535.1 | 100 | 100 | 100 | Severe acute respiratory syndrome coronavirus 2 |
| | | | | isolate SARS-CoV-2/human/USA/UT- |
| | | | | 00016/2020, complete genome |
| MT334534.1 | 100 | 100 | 100 | Severe acute respiratory syndrome coronavirus 2 |
| 0 | | | | isolate SARS-CoV-2/human/USA/UT- |
| | | | | 0015/2020, complete genome |
| MT334533.1 | 100 | 100 | 100 | Severe acute respiratory syndrome coronavirus 2 |
| | | | | isolate SARS-CoV-2/human/USA/UT- |
| | | | | 00014/2020, complete genome |
| MT334532.1 | 100 | 100 | 100 | Severe acute respiratory syndrome coronavirus 2 |
| | | | | isolate SARS-CoV-2/human/USA/UT- |
| | | | | 00013/2020 ORF1ab polyprotein (ORF1ab), |
| | | | | ORF1a polyprotein (ORF1ab), surface |
| | | | | glycoprotein (S), ORF3a protein (ORF3a), |
| | | | | envelope protein (E), membrane glycoprotein |
| | | | | (M), ORF6 protein (ORF6), ORF7a protein |
| | | | | (ORF7a), ORF7b (ORF7b), ORF8 protein |
| | | | | (ORF8), nucleocapsid phosphoprotein (N), and |
| | | | | ORF10 protein (ORF10) genes, complete cds |
| MT334531.1 | 100 | 100 | 100 | Severe acute respiratory syndrome coronavirus 2 |
| | | | | isolate SARS-CoV-2/human/USA/UT- |
| | | | | 00012/2020, complete genome |
| MT334530.1 | 100 | 100 | 100 | Severe acute respiratory syndrome coronavirus 2 |
| | | | | isolate SARS-CoV-2/human/USA/UT- |
| | | | | 00011/2020 ORF1ab polyprotein (ORF1ab), |
| | | | | ORF1a polyprotein (ORF1ab), surface |
| | | | | glycoprotein (S), ORF3a protein (ORF3a), |
| | | | | envelope protein (E), membrane glycoprotein |
| | | | | (M), ORF6 protein (ORF6), ORF7a protein |
| | | | | (ORF7a), ORF7b (ORF7b), ORF8 protein |
| | | | | (ORF8), nucleocapsid phosphoprotein (N), and |
| | | | | ORF10 protein (ORF10) genes, complete cds |
| MT334529.1 | 100 | 100 | 100 | Severe acute respiratory syndrome coronavirus 2 |
| | | | | isolate SARS-CoV-2/human/USA/UT- |
| | | | | 00010/2020, complete genome |
| MT334528.1 | 100 | 100 | 100 | Severe acute respiratory syndrome coronavirus 2 |
| | | | | isolate SARS-CoV-2/human/USA/UT- |
| | | | | 00009/2020 ORF1ab polyprotein (ORF1ab), |
| | | | | ORF1a polyprotein (ORF1ab), surface |
| | | | | glycoprotein (S), ORF3a protein (ORF3a), |
| | | | | envelope protein (E), membrane glycoprotein |
| | | | | (M), and ORF6 protein (ORF6) genes, complete |
| | | | | cds; ORF7a protein (ORF7a) and ORF7b |
| | | | | (ORF7b) genes, partial cds; and ORF8 protein |
| | | | | (ORF8), nucleocapsid phosphoprotein (N), and |
| | | | | ORF10 protein (ORF10) genes, complete cds |
| MT334527.1 | 100 | 100 | 100 | Severe acute respiratory syndrome coronavirus 2 |
| | | | | isolate SARS-CoV-2/human/USA/UT- |
| | | | | 00008/2020 ORF1ab polyprotein (ORF1ab), |
| | | | | ORF1a polyprotein (ORF1ab), surface |
| | | | | glycoprotein (S), ORF3a protein (ORF3a), |
| | | | | envelope protein (E), membrane glycoprotein |
| | | | | (M), ORF6 protein (ORF6), ORF7a protein |
| | | | | (ORF7a), ORF7b (ORF7b), ORF8 protein |
| | | | | (ORF8), nucleocapsid phosphoprotein (N), and |
| | | | | ORF10 protein (ORF10) genes, complete cds |
| MT334526.1 | 100 | 100 | 100 | Severe acute respiratory syndrome coronavirus 2 |
| | | | | isolate SARS-CoV-2/human/USA/UT- |
| | | | | 00006/2020, complete genome |
| MT334525.1 | 100 | 100 | 100 | Severe acute respiratory syndrome coronavirus 2 |
| | | | | isolate SARS-CoV-2/human/USA/UT- |
| | | | | 00005/2020 ORF1ab polyprotein (ORF1ab), |
| | | | | ORF1a polyprotein (ORF1ab), surface |
| | | | | glycoprotein (S), ORF3a protein (ORF3a), |
| | | | | envelope protein (E), membrane glycoprotein |
| | | | | (M), ORF6 protein (ORF6), ORF7a protein |
| | | | | (ORF7a), ORF7b (ORF7b), ORF8 protein |
| | | | | (ORF8), nucleocapsid phosphoprotein (N), and |
| | | | | ORF10 protein (ORF10) genes, complete cds |
| MT334524.1 | 100 | 100 | 100 | Severe acute respiratory syndrome coronavirus 2 |
| | | | | isolate SARS-CoV-2/human/USA/UT- |
| | | | | 00004/2020 ORF1ab polyprotein (ORF1ab), |
| | | | | ORF1a polyprotein (ORF1ab), surface |
| | | | | glycoprotein (S), ORF3a protein (ORF3a), |
| | | | | envelope protein (E), membrane glycoprotein |
| | | | | (M), ORF6 protein (ORF6), ORF7a protein |
| | | | | (ORF7a), ORF7b (ORF7b), ORF8 protein |
| | | | | (ORF8), nucleocapsid phosphoprotein (N), and |
| | | | | ORF10 protein (ORF10) genes, complete cds |
| MT334523.1 | 100 | 100 | 100 | Severe acute respiratory syndrome coronavirus 2 |
| | | | | isolate SARS-CoV-2/human/USA/UT- |
| | | | | 00003/2020, complete genome |
| MT334522.1 | 100 | 100 | 100 | Severe acute respiratory syndrome coronavirus 2 |
| | | | | isolate SARS-CoV-2/human/USA/UT- |
| | | | | 00001/2020 ORF1ab polyprotein (ORF1ab), |
| | | | | ORF1a polyprotein (ORF1ab), surface |
| | | | | glycoprotein (S), ORF3a protein (ORF3a), |
| | | | | envelope protein (E), membrane glycoprotein |
| | | | | (M), ORF6 protein (ORF6), ORF7a protein |
| | | | | (ORF7a), ORF7b (ORF7b), ORF8 protein |
| | | | | (ORF8), nucleocapsid phosphoprotein (N), and |
| | | | | ORF10 protein (ORF10) genes, complete cds |
| MT339041.1 | 100 | 100 | 100 | Severe acute respiratory syndrome coronavirus 2 |
| | | | | isolate SARS-CoV-2/human/USA/AZ- |
| | | | | A5U2936/2020, complete genome |
| MT339039.1 | 100 | 100 | 100 | Severe acute respiratory syndrome coronavirus 2 |
| | | | | isolate SARS-CoV-2/human/USA/AZ- |
| | | | | ASU2922/2020, complete genome |
| NC045512. | 100 | 100 | 100 | Severe acutt, respiratory syndrome coronavirus 2 |
| 2 | | | | isolate Wuhan-Flu-1, complete genome |
| MT253710.1 | 100 | 100 | 96.88 | Severe acute respiratory syndrome coronavirus 2 |
| | | | | isolate SARS-CoV-2/humanICH/HZ-91/2020, |
| | | | | complete genome |
| MT198652.2 | 100 | 100 | 100 | Severe acute respiratory syndrome coronavirus 2 |
| | | | | isolate SARS-CoV- |
| | | | | 2/human/ESPNalencia003/2020, complete |
| | | | | genome |
| MT334549.1 | 100 | 100 | 100 | Severe acute respiratory syndrome coronavirus 2 |
| | | | | isolate SARS-CoV-2/human/USA/UT- |
| | | | | 00087/2020 ORF1ab polyprotein (ORF1ab), |
| | | | | ORF1a polyprotein (ORF1ab), surface |
| | | | | glycoprotein (S), ORF3a protein (ORF3a), |
| | | | | envelope protein (E), and membrane |
| | | | | glycoprotein (M) genes, partial cds; ORF6 |
| | | | | protein (ORF6) gene, complete cds; ORF7a |
| | | | | protein (ORF7a) gene, partial cds; ORF7b gene, |
| | | | | complete sequence; ORF8 protein (ORF8) gene, |
| | | | | partial cds; and nucleocapsid phosphoprotein (N) |
| | | | | and ORF10 protein (ORF10) genes, complete |
| | | | | cds |
| MT325579.1 | 100 | 100 | 100 | Severe acute respiratory syndrome coronavirus 2 |
| | | | | isolate SARS-CoV- |
| | | | | 2/human/USA/IN2001/2020, complete genome |

3.-3) COVID-19_RdRP Assay: (RdRPgene; GenBank accession: MN9089473):

| | | | | |
|---|---|---|---|---|
| MT345832.1 | 100 | 100 | 100 | Severe acute respiratory syndrome coronavirus 2 isolate |
| | | | | SARS-CoV-2/human/USA/WA-UW-2181/2020, |
| | | | | complete genome |
| MT345831.1 | 100 | 100 | 100 | Severe acute respiratory syndrome coronavirus 2 isolate |
| | | | | SARS-CoV-2/human/USA/ID-UW-2159/2020 ORF1ab |
| | | | | polyprotein (ORF1ab) and ORF1a polyprotein, nspll |
| | | | | region, (ORF1ab) genes, partial cds; and surface |
| | | | | glycoprotein (S), ORF3a protein (ORF3a), envelope |
| | | | | protein (E), membrane glycoprotein (M), ORF6 protein |
| | | | | (ORF6), ORF7a protein (ORF7a), ORF7b(ORF7b), |
| | | | | ORF8 protein (ORF8), nucleocapsid phosphoprotein (N), |
| | | | | and ORF10 protein (ORF10) genes, complete cds |
| MT345830.1 | 100 | 100 | 100 | Severe acute respiratory syndrome coronavirus 2 isolate |
| | | | | SARS-CoV-2/human/USA/WA-UW-2149/2020, |
| | | | | complete genome |
| MT345829.1 | 100 | 100 | 100 | Severe acute respiratory syndrome coronavirus 2 isolate |
| | | | | SARS-CoV-2/human/USA/WA-UW-2142/2020, |
| | | | | complete genome |
| M1345828.1 | 100 | 100 | 100 | Severe acute respiratory syndrome coronavirus 2 isolate |
| | | | | SARS-CoV-2/human/USA/ID-UW-2160/2020, complete |
| | | | | genome |
| MT345827.1 | 98.3 | 100 | 100 | Severe acute respiratory syndrome coronavirus 2 isolate |
| | | | | SARS-CoV-2/human/USA/WA-UW-2146/2020, |
| | | | | complete genome |
| MT345826.1 | 100 | 100 | 100 | Severe acute respiratory syndrome coronavirus 2 isolate |
| | | | | SARS-CoV-2/human/USA/WA-UW-2144/2020, |
| | | | | complete genome |
| MT345825.1 | 100 | 100 | 100 | Severe acute respiratory syndrome coronavirus 2 isolate |
| | | | | SARS-CoV-2/human/USA/WA-UW-2145/2020, |
| | | | | complete genome |
| MT345824.1 | 100 | 100 | 100 | Severe acute respiratory syndrome coronavirus 2 isolate |
| | | | | SARS-CoV-2/human/USA/WA-UW-2140/2020, |
| | | | | complete genome |
| MT345823.1 | 100 | 100 | 100 | Severe acute respiratory syndrome coronavirus 2 isolate |
| | | | | SARS-CoV-2/human/USA/WA-UW-2164/2020, |
| | | | | complete genome |
| MT345822.1 | 100 | 100 | 100 | Severe acute respiratory syndrome coronavirus 2 isolate |
| | | | | SARS-CoV-2/human/USA/WA-UW-2141/2020, |
| | | | | complete genome |
| MT345821.1 | 100 | 100 | 100 | Severe acute respiratory syndrome coronavirus 2 isolate |
| | | | | SARS-CoV-2/human/USA/WA-UW-2100/2020, |
| | | | | complete genome |
| MT345820.1 | 100 | 100 | 100 | Severe acute respiratory syndrome coronavirus 2 isolate |
| | | | | SARS-CoV-2/human/USA/WA-UW-2129/2020 ORF1ab |
| | | | | polyprotein (ORF1ab) and ORF1a polyprotein (ORF1ab) |
| | | | | genes, partial cds; and surface glycoprotein (S), ORF3a |
| | | | | protein (ORF3a), envelope protein (E), membrane |
| | | | | glycoprotein (M), ORF6 protein (ORF6), ORF7a protein |
| | | | | (ORF7a), ORF7b (ORF7b), ORF8 protein (ORF8), |
| | | | | nucleocapsid phosphoprotein (N), and ORF10 protein |
| | | | | (ORF10) genes, complete cds |
| MT345819.1 | 100 | 100 | 100 | Severe acute respiratory syndrome coronavirus 2 isolate |
| | | | | SARS-CoV-2/human/USA/WA-UW-2112/2020, |
| | | | | complete genome |
| MT345818.1 | 100 | 100 | 100 | Severe acute respiratory syndrome coronavirus 2 isolate |
| | | | | SARS-CoV-2/human/USA/WA-UW-2109/2020 ORF1ab |
| | | | | polyprotein (ORF1ab) and ORF1a polyprotein (ORF1ab) |
| | | | | genes, partial cds; and surface glycoprotein (S), ORF3a |
| | | | | protein (ORF3a), envelope protein (E), membrane |
| | | | | glycoprotein (M), ORF6 protein (ORF6), ORF7a protein |
| | | | | (ORF7a), ORF7b (ORF7b), ORF8 protein (ORF8), |
| | | | | nucleocapsid phosphoprotein (N), and ORF10 protein |
| | | | | (ORF10) genes, complete cds |
| M1345817.1 | 100 | 100 | 100 | Severe acute respiratory syndrome coronavirus 2 isolate |
| | | | | SARS-CoV-2/human/USA/ID-UW-2134/2020, complete |
| | | | | genome |
| MT345816.1 | 100 | 100 | 100 | Severe acute respiratory syndrome coronavirus 2 isolate |
| | | | | SARS-CoV-2/human/USA/UNKNOWN-UW- |
| | | | | 2139/2020, complete genome |
| MT345815.1 | 100 | 100 | 100 | Severe acute respiratory syndrome coronavirus 2 isolate |
| | | | | SARS-CoV-2/human/USA/WA-UW-2137/2020, |
| | | | | complete genome |
| MT345814.1 | 100 | 100 | 100 | Severe acute respiratory syndrome coronavirus 2 isolate |
| | | | | SARS-CoV-2/human/USA/WA-UW-2104/2020, |
| | | | | complete genome |
| MT345813.1 | 100 | 100 | 100 | Severe acute respiratory syndrome coronavirus 2 isolate |
| | | | | SARS-CoV-2/human/USA/WA-UW-2115/2020 ORF1ab |
| | | | | polyprotein (ORF1ab) and ORF1a polyprotein (ORF1ab) |
| | | | | genes, partial cds; and surface glycoprotein (S), ORF3a |
| | | | | protein (ORF3a), envelope protein (E), membrane |
| | | | | glycoprotein (M), ORF6 protein (ORF6), ORF7a protein |
| | | | | (ORF7a), ORF7b (ORF7b), ORF8 protein (ORF8), |
| | | | | nucleocapsid phosphoprotein (N), and ORF10 protein |
| | | | | (ORF10) genes, complete cds |
| MT345812.1 | 100 | 100 | 100 | Severe acute respiratory syndrome coronavirus 2 isolate |
| | | | | SARS-CoV-2/human/USA/WA-UW-2094/2020, |
| | | | | complete genome |
| MT345811.1 | 100 | 100 | 100 | Severe acute respiratory syndrome coronavirus 2 isolate |
| | | | | SARS-CoV-2/human/USA/WA-UW-2108/2020, |
| | | | | complete genome |
| MT345810.1 | 100 | 100 | 100 | Severe acute respiratory syndrome coronavirus 2 isolate |
| | | | | SARS-CoV-2/human/USA/WA-UW-2086/2020ORF1ab |
| | | | | polyprotein (ORF1ab) and ORF1a polyprotein (ORF1ab) |
| | | | | genes, partial cds; and surface glycoprotein (S), ORF3a |
| | | | | protein (ORF3a), envelope protein (E), membrane |
| | | | | glycoprotein (M), ORF6 protein (ORF6), ORF7a protein |
| | | | | (ORF7a), ORF7b (ORF7b), ORF8 protein (ORF8), |
| | | | | nucleocapsid phosphoprotein (N), and ORF10 protein |
| | | | | (ORF10) genes, complete cds |
| MT345809.1 | 100 | 100 | 100 | Severe acute respiratory syndrome coronavirus 2 isolate |
| | | | | SARS-CoV-2/human/USA/WA-UW-2119/2020, |
| | | | | complete genome |
| MT345808.1 | 100 | 100 | 100 | Severe acute respiratory syndrome coronavirus 2 isolate |
| | | | | SARS-CoV-2/human/USA/WA-UW-2132/2020, |
| | | | | complete genome |
| MT345807.1 | 100 | 100 | 100 | Severe acute respiratory syndrome coronavirus 2 isolate |
| | | | | SARS-CoV-2/human/USA/WA-UW-2074/2020 ORF1ab |
| | | | | polyprotein (ORF1ab) and ORF1a polyprotein (ORF1ab) |
| | | | | genes, partial cds; and surface glycoprotein (S), ORF3a |
| | | | | protein (ORF3a), envelope protein (E), membrane |
| | | | | glycoprotein (M), ORF6 protein (ORF6), ORF7a protein |
| | | | | (ORF7a), ORF7b (ORF7b), ORF8 protein (ORF8), |
| | | | | nucleocapsid phosphoprotein (N), and ORF10 protein |
| | | | | (ORF10) genes, complete cds |
| MT345806.1 | 100 | 100 | 100 | Severe acute respiratory syndrome coronavirus 2 isolate |
| | | | | SARS-CoV-2/human/USA/WA-UW-2087/2020, |
| | | | | complete genome |
| MT345805.1 | 100 | 100 | 100 | Severe acute respiratory syndrome coronavirus 2 isolate |
| | | | | SARS-CoV-2/human/USA/WA-UW-2078/2020, |
| | | | | complete genome |
| MT345804.1 | 100 | 100 | 100 | Severe acute respiratory syndrome coronavirus 2 isolate |
| | | | | SARS-CoV-2/human/USA/WA-UW-2072/2020ORF1ab |
| | | | | polyprotein (ORF1ab) and ORF1a polyprotein (ORF1ab) |
| | | | | genes, partial cds; and surface glycoprotein (S), ORF3a |
| | | | | protein (ORF3a), envelope protein (E), membrane |
| | | | | glycoprotein (M), ORF6 protein (ORF6), ORF7a protein |
| | | | | (ORF7a), ORF7b (ORF7b), ORF8 protein (ORF8), |
| | | | | nucleocapsid phosphoprotein (N), andORF10 protein |
| | | | | (ORF10) genes, complete cds |
| M1345803.1 | 100 | 100 | 100 | Severe acute respiratory syndrome coronavirus 2 isolate |
| | | | | SARS-CoV-2/human/USA/ID-UW-2127/2020, complete |
| | | | | genome |
| MT345802.1 | 100 | 100 | 100 | Severe acute respiratory syndrome coronavirus 2 isolate |
| | | | | SARS-CoV-2/human/USA/UNKNOWN-UW- |
| | | | | 2069/2020, complete genome |
| MT345801.1 | 100 | 100 | 100 | Severe acute respiratory syndrome coronavirus 2 isolate |
| | | | | SARS-CoV-2/human/USA/WA-UW-2131/2020, |
| | | | | complete genome |
| MT345800.1 | 100 | 100 | 100 | Severe acute respiratory syndrome coronavirus 2 isolate |
| | | | | SARS-CoV-2/human/USA/WA-UW-2082/2020ORF1ab |
| | | | | polyprotein (ORF1ab), ORF1a polyprotein (ORF1ab), |
| | | | | surface glycoprotein (S), ORF3a protein (ORF3a), |
| | | | | envelope protein (E), membrane glycoprotein (M), ORF6 |
| | | | | protein (ORF6), ORF7a protein (ORF7a), ORF7b |
| | | | | (ORF7b), ORF8 protein (ORF8), nucleocapsid |
| | | | | phosphoprotein(N), and ORF10 protein (ORF10) genes, |
| | | | | complete cds |
| MT345799.1 | 100 | 100 | 100 | Severe acute respiratory syndrome coronavirus 2 isolate |
| | | | | SARS-CoV-2/human/USA/WA-UW-2169/2020, |
| | | | | complete genome |
| MT345798.1 | 100 | 100 | 100 | Severe acute respiratory syndrome coronavirus 2 isolate |
| | | | | SARS-CoV-2/human/USA/WA-UW-2091/2020, |
| | | | | complete genome |
| MT344963.1 | 100 | 100 | 100 | Severe acute respiratory syndrome coronavirus 2 isolate |
| | | | | SARS-CoV-2/human/USA/NC_0025/2020, complete |
| | | | | genome |
| MT344962.1 | 100 | 100 | 100 | Severe acute respiratory syndrome coronavirus 2 isolate |
| | | | | SARS-CoV-2/human/USA/MN_0101/2020, complete |
| | | | | genome |
| M1344961.1 | 100 | 100 | 100 | Severe acute respiratory syndrome coronavirus 2 isolate |
| | | | | SARS-CoV-2/human/USA/NV_0016/2020, complete |
| | | | | genome |
| MT344960.1 | 100 | 100 | 100 | Severe acute respiratory syndrome coronavirus 2 isolate |
| | | | | SARS-CoV-2/human/USA/RI_0882/2020, complete |
| | | | | genome |
| MT344959.1 | 100 | 100 | 100 | Severe acute respiratory syndrome coronavirus 2 isolate |
| | | | | SARS-CoV-2/human/USA/PA_2887/2020, complete |
| | | | | genome |
| MT344958.1 | 100 | 100 | 100 | Severe acute respiratory syndrome coronavirus 2 isolate |
| | | | | SARS-CoV-2/human/USA/PA_2735/2020, complete |
| | | | | genome |
| MT344957.1 | 100 | 100 | 100 | Severe acute respiratory syndrome coronavirus 2 isolate |
| | | | | SARS-CoV-2/human/USA/PA_2734/2020, complete |
| | | | | genome |
| MT344956.1 | 100 | 100 | 100 | Severe acute respiratory syndrome coronavirus 2 isolate |
| | | | | SARS-CoV-2/human/USA/OH_0020/2020, complete |
| | | | | genome |
| M1344955.1 | 100 | 100 | 100 | Severe acute respiratory syndrome coronavirus 2 isolate |
| | | | | SARS-CoV-2/human/USA/OH_0019/2020, complete |
| | | | | genome |
| M1344954.1 | 100 | 100 | 100 | Severe acute respiratory syndrome coronavirus 2 isolate |
| | | | | SARS-CoV-2/human/USA/MN_0100/2020, complete |
| | | | | genome |
| M1344953.1 | 100 | 100 | 100 | Severe acute respiratory syndrome coronavirus 2 isolate |
| | | | | SARS-CoV-2/human/USA/MD_0027/2020, complete |
| | | | | genome |
| M1344952.1 | 100 | 100 | 100 | Severe acute respiratory syndrome coronavirus 2 isolate |
| | | | | SARS-CoV-2/human/USA/IN_92003/2020, complete |
| | | | | genome |
| MT344951.1 | 100 | 100 | 100 | Severe acute respiratory syndrome coronavirus 2 isolate |
| | | | | SARS-CoV-2/human/USA/IN_82003/2020, complete |
| | | | | genome |
| MT344950.1 | 100 | 100 | 100 | Severe acute respiratory syndrome coronavirus 2 isolate |
| | | | | SARS-CoV-2/human/USA/IN_72003/2020, complete |
| | | | | genome |
| MT344949.1 | 100 | 100 | 100 | Severe acute respiratory syndrome coronavirus 2 isolate |
| | | | | SARS-CoV-2/human/USA/HI_7881/2020, complete |
| | | | | genome |
| M1344948.1 | 100 | 100 | 100 | Severe acute respiratory syndrome coronavirus 2 isolate |
| | | | | SARS-CoV-2/human/USA/HI_4970/2020, complete |
| | | | | genome |
| MT344947.1 | 100 | 100 | 100 | Severe acute respiratory syndrome coronavirus 2 isolate |
| | | | | SARS-CoV-2/human/USA/GA_2275/2020, complete |
| | | | | genome |
| MT344946.1 | 100 | 100 | 100 | Severe acute respiratory syndrome coronavirus 2 isolate |
| | | | | SARS-CoV-2/human/USA/GA_2147/2020, complete |
| | | | | genome |
| MT344945.1 | 100 | 100 | 100 | Severe acute respiratory syndrome coronavirus 2 isolate |
| | | | | SARS-CoV-2/human/USA/GA_2070/2020, complete |
| | | | | genome |
| MT344944.1 | 100 | 100 | 100 | Severe acute respiratory syndrome coronavirus 2 isolate |
| | | | | SARS-CoV-2/human/USA/CT_9849/2020, complete |
| | | | | genome |
| M1350282.1 | 100 | 100 | 100 | Severe acute respiratory syndrome coronavirus 2 isolate |
| | | | | SARS-CoV-2/human/BRA/SP02cc/2020, complete |
| | | | | genome |
| MT350280.1 | 100 | 100 | 100 | Severe acute respiratory syndrome coronavirus 2 isolate |
| | | | | SARS-CoV-2/human/USA/USA-WA_0447/2020, |
| | | | | complete genome |
| MT350279.1 | 100 | 100 | 100 | Severe acute respiratory syndrome coronavirus 2 isolate |
| | | | | SARS-CoV-2/human/USA/USA-WA_0711/2020, |
| | | | | complete genome |
| MT350278.1 | 100 | 100 | 100 | Severe acute respiratory syndrome coronavirus 2 isolate |
| | | | | SARS-CoV-2/human/USA/USA-WA_0440/2020, |
| | | | | complete genome |
| MT350277.1 | 100 | 100 | 100 | Severe acute respiratory syndrome coronavirus 2 isolate |
| | | | | SARS-CoV-2/human/USA/USA-WA_0441/2020, |
| | | | | complete genome |
| MT350276.1 | 100 | 100 | 100 | Severe acute respiratory syndrome coronavirus 2 isolate |
| | | | | SARS-CoV-2/human/USA/USA-WA_0429/2020, |
| | | | | complete genome |
| MT350275.1 | 100 | 100 | 100 | Severe acute respiratory syndrome coronavirus 2 isolate |
| | | | | SARS-CoV-2/human/USA/USA-WA_0484/2020, |
| | | | | complete genome |
| MT350274.1 | 100 | 100 | 100 | Severe acute respiratory syndrome coronavirus 2 isolate |
| | | | | SARS-CoV-2/human/USA/USA-WA_0422/2020, |
| | | | | complete genome |
| MT350273.1 | 100 | 100 | 100 | Severe acute respiratory syndrome coronavirus 2 isolate |
| | | | | SARS-CoV-2/human/USA/USA-WA_0478/2020, |
| | | | | complete genome |
| MT350270.1 | 100 | 100 | 100 | Severe acute respiratory syndrome coronavirus 2 isolate |
| | | | | SARS-CoV-2/human/USA/USA-WA0432/2020, |
| | | | | complete genome |
| MT350269.1 | 100 | 100 | 100 | Severe acute respiratory syndrome coronavirus 2 isolate |
| | | | | SARS-CoV-2/human/USA/USA-WA0797/2020, |
| | | | | complete genome |
| MT350268.1 | 100 | 100 | 100 | Severe acute respiratory syndrome coronavirus 2 isolate |
| | | | | SARS-CoV-2/human/USA/USA-WA_0418/2020, |
| | | | | complete genome |
| MT350267.1 | 100 | 100 | 100 | Severe acute respiratory syndrome coronavirus 2 isolate |
| | | | | SARS-CoV-2/human/USA/USA-WA_0442/2020, |
| | | | | complete genome |
| MT350266.1 | 100 | 100 | 100 | Severe acute respiratory syndrome coronavirus 2 isolate |
| | | | | SARS-CoV-2/human/USA/USA-WA_0434/2020, |
| | | | | complete genome |
| MT350265.1 | 100 | 100 | 100 | Severe acute respiratory syndrome coronavirus 2 isolate |
| | | | | SARS-CoV-2/human/USA/USA-WA_0427/2020, |
| | | | | complete genome |
| MT350264.1 | 100 | 100 | 100 | Severe acute respiratory syndrome coronavirus 2 isolate |
| | | | | SARS-CoV-2/human/USA/USA-WA_0428/2020, |
| | | | | complete genome |
| MT350263.1 | 100 | 100 | 100 | Severe acute respiratory syndrome coronavirus 2 isolate |
| | | | | SARS-CoV-2/human/USA/USA-WA_0480/2020, |
| | | | | complete genome |
| MT350257.1 | 100 | 100 | 100 | Severe acute respiratory syndrome coronavirus 2 isolate |
| | | | | SARS-CoV-2/human/USANA-DCLS-0068/2020, |
| | | | | complete genome |
| MT350256.1 | 100 | 100 | 100 | Severe acute respiratory syndrome coronavirus 2 isolate |
| | | | | SARS-CoV-2/human/USANA-DCLS-0067/2020, |
| | | | | complete genome |
| MT350255.1 | 100 | 100 | 100 | Severe acute respiratory syndrome coronavirus 2 isolate |
| | | | | SARS-CoV-2/human/USANA-DCLS-0066/2020, |
| | | | | complete genome |
| MT350254.1 | 100 | 100 | 100 | Severe acute respiratory syndrome coronavirus 2 isolate |
| | | | | SARS-CoV-2/human/USANA-DCLS-0065/2020, |
| | | | | complete genome |
| MT350253.1 | 100 | 100 | 100 | Severe acute respiratory syndrome coronavirus 2 isolate |
| | | | | SARS-CoV-2/human/USANA-DCLS-0064/2020, |
| | | | | complete genome |
| MT350252.1 | 100 | 100 | 100 | Severe acute respiratory syndrome coronavirus 2 isolate |
| | | | | SARS-CoV-2/human/USANA-DCLS-0063/2020, |
| | | | | complete genome |
| MT350251.1 | 100 | 100 | 100 | Severe acute respiratory syndrome coronavirus 2 isolate |
| | | | | SARS-CoV-2/human/USANA-DCLS-0061/2020, |
| | | | | complete genome |
| MT350250.1 | 100 | 100 | 100 | Severe acute respiratory syndrome coronavirus 2 isolate |
| | | | | SARS-CoV-2/human/USANA-DCLS-0060/2020, |
| | | | | complete genome |
| MT350249.1 | 100 | 100 | 100 | Severe acute respiratory syndrome coronavirus 2 isolate |
| | | | | SARS-CoV-2/human/USANA-DCLS-0059/2020, |
| | | | | complete genome |
| MT350248.1 | 100 | 100 | 100 | Severe acute respiratory syndrome coronavirus 2 isolate |
| | | | | SARS-CoV-2/human/USANA-DCLS-0058/2020, |
| | | | | complete genome |
| MT350247.1 | 100 | 100 | 100 | Severe acute respiratory syndrome coronavirus 2 isolate |
| | | | | SARS-CoV-2/human/USANA-DCLS-0057/2020, |
| | | | | complete genome |
| MT350246.1 | 100 | 100 | 100 | Severe acute respiratory syndrome coronavirus 2 isolate |
| | | | | SARS-CoV-2/human/USANA-DCLS-0056/2020, |
| | | | | complete genome |
| MT350245.1 | 100 | 100 | 100 | Severe acute respiratory syndrome coronavirus 2 isolate |
| | | | | SARS-CoV-2/human/USANA-DCLS-0055/2020, |
| | | | | complete genome |
| MT350244.1 | 100 | 100 | 100 | Severe acute respiratory syndrome coronavirus 2 isolate |
| | | | | SARS-CoV-2/human/USANA-DCLS-0054/2020, |
| | | | | complete genome |
| MT350243.1 | 100 | 100 | 100 | Severe acute respiratory syndrome coronavirus 2 isolate |
| | | | | SARS-CoV-2/human/USANA-DCLS-0053/2020, |
| | | | | complete genome |
| MT350242.1 | 100 | 100 | 100 | Severe acute respiratory syndrome coronavirus 2 isolate |
| | | | | SARS-CoV-2/human/USANA-DCLS-0052/2020, |
| | | | | complete genome |
| MT350241.1 | 100 | 100 | 100 | Severe acute respiratory syndrome coronavirus 2 isolate |
| | | | | SARS-CoV-2/human/USANA-DCLS-0051/2020, |
| | | | | complete genome |
| MT350240.1 | 100 | 100 | 100 | Severe acute respiratory syndrome coronavirus 2 isolate |
| | | | | SARS-CoV-2/human/USANA-DCLS-0050/2020, |
| | | | | complete genome |
| MT350239.1 | 100 | 100 | 100 | Severe acute respiratory syndrome coronavirus 2 isolate |
| | | | | SARS-CoV-2/human/USANA-DCLS-0049/2020, |
| | | | | complete genome |
| MT350238.1 | 100 | 100 | 100 | Severe acute respiratory syndrome coronavirus 2 isolate |
| | | | | SARS-CoV-2/human/USANA-DCLS-0048/2020, |
| | | | | complete genome |
| MT350237.1 | 100 | 100 | 100 | Severe acute respiratory syndrome coronavirus 2 isolate |
| | | | | SARS-CoV-2/human/USANA-DCLS-0047/2020, |
| | | | | complete genome |
| MT350236.1 | 100 | 100 | 100 | Severe acute respiratory syndrome coronavirus 2 isolate |
| | | | | SARS-CoV-2/human/USANA-DCLS-0041/2020, |
| | | | | complete genome |
| MT350234.1 | 100 | 100 | 100 | Severe acute respiratory syndrome coronavirus 2 |
| | | | | ORF1ab polyprotein, RNA-dependent RNA polymerase |
| | | | | region, (ORF1ab) gene, partial cds |
| NC045512.2 | 100 | 100 | 100 | Severe acute respiratory syndrome coronavirus 2 isolate |
| | | | | Wuhan-Hu-1, complete genome |
| MT334562.1 | 100 | 100 | 100 | Severe acute respiratory syndrome coronavirus 2 isolate |
| | | | | SARS-CoV-2/human/USA/UT-00302/2020 ORF1ab |
| | | | | polyprotein (ORF1ab) gene, partial cds; ORF1a |
| | | | | polyprotein (ORF1ab) gene, complete cds; surface |
| | | | | glycoprotein (S) gene, partial cds; ORF3a protein |
| | | | | (ORF3a) gene, complete cds; envelope protein (E) and |
| | | | | membrane glycoprotein (M) genes, partial cds; ORF6 |
| | | | | protein (ORF6) gene, complete cds; ORF7a protein |
| | | | | (ORF7a) and ORF7b (ORF7b) genes, partial cds; ORF8 |
| | | | | protein (ORF8) gene, complete cds; nucleocapsid |
| | | | | phosphoprotein (N) gene, partial cds; and ORF10 gene, |
| | | | | complete sequence |
| M1334561.1 | 100 | 100 | 100 | Severe acute respiratory syndrome coronavirus 2 isolate |
| | | | | SARS-CoV-2/human/USA/UT-00301/2020, complete |
| | | | | genome |
| MT334560.1 | 100 | 100 | 100 | Severe acute respiratory syndrome coronavirus 2 isolate |
| | | | | SARS-CoV-2/human/USA/UT-00300/2020 ORF1ab |
| | | | | polyprotein (ORF1ab), ORF1a polyprotein (ORF1ab), |
| | | | | surface glycoprotein (S), ORF3a protein (ORF3a), |
| | | | | envelope protein (E), membrane glycoprotein (M), and |
| | | | | ORF6 protein (ORF6) genes, complete cds; ORF7a gene, |
| | | | | complete sequence; and ORF7b(ORF7b), ORF8 protein |
| | | | | (ORF8), nucleocapsid phosphoprotein (N), and ORF10 |
| | | | | protein (ORF10) genes, complete cds |
| MT334559.1 | 100 | 100 | 100 | Severe acute respiratory syndrome coronavirus 2 isolate |
| | | | | SARS-CoV-2/human/USA/UT-00297/2020 ORF1ab |
| | | | | polyprotein (ORF1ab), ORF1a polyprotein (ORF1ab), |
| | | | | surface glycoprotein (S), ORF3a protein (ORF3a), |
| | | | | envelope protein (E), membrane glycoprotein (M), ORF6 |
| | | | | protein (ORF6), ORF7a protein (ORF7a), ORF7b |
| | | | | (ORF7b), ORF8 protein (ORF8), nucleocapsid |
| | | | | phosphoprotein (N), and ORF10 protein (ORF10) genes, |
| | | | | complete cds |

In silico cross-reactivity analysis with SARS-CoV-2 ORFlab, N and RdRP primer/probe: Several organisms are extracted and tested with the GG COVID-19 QPlex Real-Time PCR to demonstrate analytical specificity and exclusivity. Studies are performed with nucleic acids extracted using the_ instrument and_Kit. Nucleic acids are extracted from high titer preparations (typically ≥10\hat{ }5 PFU/mL or ≥10\hat{ }6 CFU/mL). Testing is performed using the QIAGEN One Step RT-PCR Kit on the Rotorgene Real-Time PCR instrument. Cross-reactivity of the GG COVID-19 QPlex RT-PCR Kit is evaluated using both in silico analysis and wet testing against normal and pathogenic organisms found in the respiratory tract. BLASTn analysis queries of the GG COVID-19 QPlex RT-PCR Kit primers and probes are performed against public domain nucleotide sequences with default settings. The database search parameters are as follows:

The nucleotide collection consists of GenBank+EMBL+DDBJ+PDB+RefSeq sequences, but excludes EST, STS, GSS, WGS, TSA, patent sequences as well as phase 0, 1, and 2 HTGS sequences and sequences longer than 100 Mb. The database is non-redundant. Identical sequences have been merged into one entry, while preserving the accession, GI, title and taxonomy information for each entry. The match and mismatch scores are 1 and −3, respectively. The penalty to create and extend a gap in an alignment is 5 and 2, respectively. The search parameters is automatically adjusted for short input sequences and the expected threshold is 1000. The BLASTn analysis indicated that no organisms, including other related SARS-coronaviruses, exhibits >80% homology to the forward primer, reverse primer, and probe for either the N or the Orflab target. RdRP forward primer shows >80% homology to Kenya bat coronaviruses, and RdRP reverse primer showed >80% homology to avian coronaviruses. The probe shows no significant homology with human genome, other coronaviruses, or human microflora. When combining primers and probe, there is no prediction of potential false positive RT-PCR results. The results of the in silico analysis suggest the GG COVID-19 QPlex RT-PCR kit is designed for the specific detection of SARS-CoV-2, with no expected cross reactivity to the human genome, other coronaviruses, or human microflora that would predict potential false positive RT-PCR results.

### Example 26—Nucleic Sequences Isolated and Used in Diagnostic Kit

The target marker/primers may be: an isolated nucleic acid molecule consisting essentially of the nucleic acid sequence of SEQ ID NO: 1-2 (N-2 primer, forward/reverse) or the full length complement thereof, wherein said nucleic acid molecule is 100 nucleotides or less in length; an isolated nucleic acid molecule consisting essentially of the nucleic acid sequence of SEQ ID NO: 3-4 (Orflab primer, forward/reverse), or the full length complement thereof, wherein said nucleic acid molecule is 100 nucleotides or less in length; an isolated nucleic acid molecule consisting essentially of the nucleic acid sequence of SEQ ID NO: 5-6 (RdRP primer, forward/reverse) or the full length complement thereof, wherein said nucleic acid molecule is 100 nucleotides or less in length; and/or an isolated nucleic acid molecule consisting essentially of the nucleic acid sequence of SEQ ID NO: 20-21 (N-1 primer, forward/reverse) or the full length complement thereof, wherein said nucleic acid molecule is 100 nucleotides or less in length.

The Internal control and positive control may be: an isolated nucleic acid molecule consisting essentially of the nucleic acid sequence of SEQ ID NO: 29 (Internal control-1) or the neighboring 3-4 bases plus/minus thereof, wherein said nucleic acid molecule is 10-15 nucleotides in length; an isolated nucleic acid molecule consisting essentially of the nucleic acid sequence of SEQ ID NO: 30 (Internal control-2) or the neighboring 3-4 bases plus/minus thereof, wherein said nucleic acid molecule is 10-15 nucleotides in length; and/pr an isolated nucleic acid molecule consisting essentially of the nucleic acid sequence of SEQ ID NO: 31 or SEQ ID NO: 45 (positive control) or the neighboring 4-5 bases plus/minus thereof, wherein said nucleic acid molecule is 100 nucleotides or less in length.

The human housekeeping gene marker/primer and probe may be an isolated nucleic acid molecule consisting essentially of the nucleic acid sequence of SEQ ID NO:7 or 8 (beta actin primer) and SEQ ID NO:18 (beta actin probe) wherein said nucleic acid molecule is 100 nucleotides or less in length; an isolated nucleic acid molecule consisting essentially of the nucleic acid sequence of SEQ ID NO 25 or 26 (Pbgd primer), and SEQ ID NO:27 (Pbgd probe), wherein said nucleic acid molecule is 100 nucleotides or less in length.

The probe is labeled with one of any of the fluorescent dye, such as, but not limited to: FAM(6-carboxyfluorescein), (texas red), (fluorescein), HEX(2′,4′,5′,7′-tetrachloro-6-carboxy-4,7-dichlorofluorescein), (fluorescein chlorotriazinyl), (rhodamine green), (rhodamine red), (tetramethylrhodamine), FITC(fluorescein isothiocyanate), (oregon green), (alexa fluor), JOE (6-Carboxy-4′,5′ Dichloro-2′,7′-Dimethoxyfluorescein), ROX (6-Carboxyl-X-Rhodamine), TET (Tetrachloro-Fluorescein), TRITC (tertramethylrodamine isothiocyanate), TAMRA (6-carboxytetramethyl-rhodamine), NED (N-(1-Naphthyl) ethylenediamine, (Cyanine) (thiadicarbocyanine).

The isolated nucleic acid molecule of Seq ID NO: 7, 8, or 18 may be quenched by the following quencher of TAMRA(6-carboxytetramethyl-rhodamine), BHQ1 (black hole quencher 1), BHQ2 (black hole quencher 2), BHQ3 (black hole quencher 3), NFQ (nonfluorescent quencher), (dabcyl), Eclipse, DDQ(Deep Dark Quencher), (Blackberry Quencher), Iowa black). A single sequence among Seq ID NO: 7, 8, and 18 is selected.

The isolated nucleic acid molecule of Seq 25, 26, or 27 may be used for detecting the presence of the COVID-19 virus and quencher of TAMRA(6-carboxytetramethyl-rhodamine), BHQ1 (black hole quencher 1), BHQ2 (black hole quencher 2), BHQ3 (black hole quencher 3), NFQ (nonfluorescent quencher), (dabcyl), Eclipse, DDQ(Deep Dark Quencher), (Blackberry Quencher), Iowa black). Two sequences among Seq ID NO: 7, 8, and 18 are selected.

SEQ ID NO: 19 may be used for detecting the presence of the COVID-19 virus with designated Reporter and Quencher.

SEQ ID NO: 20 may be used for detecting the presence of the COVID-19 virus with designated Reporter and Quencher.

Constituents of Seq ID NO: 7, 8, 5, 6, and 19 are used for detecting the presence of the COVID-19 virus.

Constituents of Seq ID NO: 25, 26, 5, 6, 28 are used for detecting the presence of the COVID-19 virus.

Target marker/probe may include: an isolated nucleic acid molecule consisting essentially of the nucleic acid sequence of SEQ ID NO:9 (N-1 possible probe site) or the full length complement thereof, wherein said nucleic acid molecule is 10-15 nucleotides or less in length; an isolated nucleic acid molecule consisting essentially of the nucleic acid sequence of SEQ ID NO 12 (Orflab possible probe site) or the full length complement thereof, wherein said nucleic acid molecule is 10-15 nucleotides or less in length; an isolated nucleic acid molecule consisting essentially of the nucleic acid sequence of SEQ ID NO:15 (RdRP possible probe site) or the full length complement thereof, wherein said nucleic acid molecule is 10-15 nucleotides or less in length; an isolated nucleic acid molecule consisting essentially of the nucleic acid sequence of SEQ ID NO:22, (N-2 possible probe site) or the full length complement thereof, wherein said nucleic acid molecule is 10-15 nucleotides or less in length.

The probe for Seq ID NO: 9 is labeled with one of any of the fluorescent dye, such as, but not limited to: FAM(6-carboxyfluorescein), (texas red), (fluorescein), HEX(2′,4′,5′,7′-tetrachloro-6-carboxy-4,7-dichlorofluorescein), (fluorescein chlorotriazinyl), (rhodamine green), (rhodamine red), (tetramethylrhodamine), FITC(fluorescein isothiocyanate), (oregon green), (alexa fluor), JOE (6-Carboxy-4′,5′ Dichloro-2′,7′-Dimethoxyfluorescein), ROX (6-Carboxyl-X-Rhodamine), TET (Tetrachloro-Fluorescein), and TRITC (tertramethylrodamine isothiocyanate), TAMRA (6-carboxytetramethyl-rhodamine), and NED (N-(1-Naphthyl) ethylenediamine, (Cyanine) (thiadicarbocyanine).

The probe for Seq ID NO: 2 is labeled with one of any of the fluorescent dye, such as, but not limited to: FAM(6-carboxyfluorescein), (texas red), (fluorescein), HEX(2′,4′,5′,7′-tetrachloro-6-carboxy-4,7-dichlorofluorescein), (fluorescein chlorotriazinyl), (rhodamine green), (rhodamine red), (tetramethylrhodamine), FITC(fluorescein isothiocyanate), (oregon green), (alexa fluor), JOE (6-Carboxy-4′,5′ Dichloro-2′,7′-Dimethoxyfluorescein), ROX (6-Carboxyl-X-Rhodamine), TET (Tetrachloro-Fluorescein), TRITC (tertramethylrodamine isothiocyanate), TAMRA (6-carboxytetramethyl-rhodamine), and NED (N-(1-Naphthyl) ethylenediamine, (Cyanine) (thiadicarbocyanine).

The probe for Seq ID NO: 15 is labeled with one of any of the fluorescent dye, such as, but not limited to: FAM(6-carboxyfluorescein), (texas red), (fluorescein), HEX(2′,4′,5′,7′-tetrachloro-6-carboxy-4,7-dichlorofluorescein), (fluorescein chlorotriazinyl), (rhodamine green), (rhodamine red), (tetramethylrhodamine), FITC(fluorescein isothiocyanate), (oregon green), (alexa fluor), JOE (6-Carboxy-4′,5′ Dichloro-2′,7′-Dimethoxyfluorescein), ROX (6-Carboxyl-X-Rhodamine), TET (Tetrachloro-Fluorescein), TRITC (tertramethylrodamine isothiocyanate), TAMRA (6-carboxytetramethyl-rhodamine), and NED (N-(1-Naphthyl) ethylenediamine, (Cyanine) (thiadicarbocyanine).

The probe for Seq ID NO: 22 is labeled with one of any of the fluorescent dye, such as, but not limited to: FAM(6-carboxyfluorescein), (texas red), (fluorescein), HEX(2′,4′,5′,7′-tetrachloro-6-carboxy-4,7-dichlorofluorescein), (fluorescein chlorotriazinyl), (rhodamine green), (rhodamine red), (tetramethylrhodamine), FITC(fluorescein isothiocyanate), (oregon green), (alexa fluor), JOE (6-Carboxy-4′,5′ Dichloro-2′,7′-Dimethoxyfluorescein), ROX (6-Carboxyl-X-Rhodamine), TET (Tetrachloro-Fluorescein), TRITC (tertramethylrodamine isothiocyanate), TAMRA (6-carboxytetramethyl-rhodamine), and NED (N-(1-Naphthyl) ethylenediamine, (Cyanine) (thiadicarbocyanine).

The quencher used for Seq ID NO: 9 include TAMRA(6-carboxytetramethyl-rhodamine), BHQ1 (black hole quencher 1), BHQ2 (black hole quencher 2), BHQ3 (black hole quencher 3), NFQ (nonfluorescent quencher), (dabcyl), Eclipse, DDQ(Deep Dark Quencher), (Blackberry Quencher), Iowa black).

The quencher used for Seq ID NO: 12 include TAMRA(6-carboxytetramethyl-rhodamine), BHQ1 (black hole quencher 1), BHQ2 (black hole quencher 2), BHQ3 (black hole quencher 3), NFQ (nonfluorescent quencher), (dabcyl), Eclipse, DDQ(Deep Dark Quencher), (Blackberry Quencher), Iowa black).

The quencher used for Seq ID NO: 15 include TAMRA(6-carboxytetramethyl-rhodamine), BHQ1 (black hole quencher 1), BHQ2 (black hole quencher 2), BHQ3 (black hole quencher 3), NFQ (nonfluorescent quencher), (dabcyl), Eclipse, DDQ(Deep Dark Quencher), (Blackberry Quencher), Iowa black).

The quencher used for Seq ID NO: 22 include TAMRA(6-carboxytetramethyl-rhodamine), BHQ1 (black hole quencher 1), BHQ2 (black hole quencher 2), BHQ3 (black hole quencher 3), NFQ (nonfluorescent quencher), (dabcyl), Eclipse, DDQ(Deep Dark Quencher), (Blackberry Quencher), Iowa black).

SEQ ID NO: 11 is used for detecting the presence of the COVID-19 virus with designated Reporter and Quencher.

SEQ ID NO: 14 is used for detecting the presence of the COVID-19 virus with designated Reporter and Quencher.

Seq ID NO: 17 is used for detecting the presence of the COVID-19 virus with designated Reporter and Quencher.

Seq ID NO: 24 is used for detecting the presence of the COVID-19 virus with designated Reporter and Quencher.

COVID-19 marker/primer and control set may involve the following isolated nucleic acid molecules for detecting the presence of the COVID-19 virus include: Seq ID NO: 1, 2, 29-31, and 45.

The isolated nucleic acid molecule may include Seq ID NO: 3, 4, 30-31, and 45 for detecting the presence of the COVID-19 virus.

The isolated nucleic acid molecule may include Seq ID NO: 5, 6, 30-31, and 45 for detecting the presence of the COVID-19 virus.

The isolated nucleic acid molecule may include Seq ID NO: 20, 21 for detecting the presence of the COVID-19 virus.

COVID-19 marker/primer, fluorescent probe, and control set may be an isolated nucleic acid molecule consisting of Seq ID NO: 1, 2, 11, and 30-31/45; Seq ID NO: 3, 4, 14, and 30-31/45; Seq ID NO: 5, 6, 17, and 30-31/45, Seq ID NO: 20, 21, 24, and 30-31/45; The fluorescent probe is a nucleic acid molecule used to detect the product.

In the systems and methods herein, a set of real time RT-PCR reagents is used for all PCR except Triplex-1 and another set of Real time RT-PCR reagent is used for Triplex-1), wherein the RT-PCR reagents are used within a PCR kit (reverse transcription real time polymerase chain reaction, RT real time PCR).

The methods herein include a method for identifying a subject infected with the COVID-19 virus comprising: obtaining total RNA from a biological sample obtained from the subject; reverse transcribing the total RNA to obtain cDNA; subjecting the cDNA to PCR assay using a set of primers, at least one of which primers consists of the nucleic acid sequence of SEQ ID 1-8 (with or without Seq ID NO: 20/21, 25/26); detecting a product of PCR assay; and wherein said detecting indicates that the subject is infected with COVID-19 virus.

The PCR kit herein may apply a set of steps, temperature, and time (reverse transcription real time polymerase chain reaction, RT real time PCR) based on: Single PCR for RdRP; Duplex PCR; RdRp and internal control (IC); Orflab and IC; N-2 and IC.

PCR kit herein may apply a set of steps, temperature, and time (reverse transcription real time polymerase chain reaction, RT real time PCR) based on: Triplex PCR; ORFlab, N, IC (Triplex-1 Real time RT-PCR); RdRP, N, and IC (Triplex-2 Real time RT-PCR); and Quadplex PCR.

A method/kit for identifying a subject infected with the COVID-19 virus, in which the specimen is applied on a variety of respiratory specimens such as nasopharyngeal, oropharyngeal swab, non-induced/correctly collected sputum, nasopharyngeal aspirate, bronchoalveolar lavage, bronchial washing, tracheal aspirate, transtracheal aspirate, and transbronchial biopsy.

The systems and methods herein also include the following below.

SEQ ID NO: 1-SEQ ID NO: 45 are described further as follows and presented in the attached sequence listing, which is incorporated in its entirety.

[primers]
SEQ ID NO: 1 - COVID-19 (SARS-Cov-2) N2 forward primer of the oligo primers (N1 forward primer)
5′- CAACTCCAGGCAGTAGG- 3′(20-mer, nt 28863-28882, MN908947.3)
SEQ ID NO: 2 - COVID-19 (SARS-Cov-2) N2 reverse primer of the oligo primers (N1 reverse primer)
5′- CCAGACATTTTGCTCTCAAGC-3′(21-mer, nt 28960-28980, MN908947.3)
SEQ ID NO: 3 - COVID-19 (SARS-Cov-2) Orf1ab forward primer of the oligo primers (ORF1ab forward
primer)
5′- GGGTTTTACACTTAAAAACACAGTC- 3′(25-mer, nt 13348-13372, M908947.3)
SEQ ID NO: 4 - COVID-19 (SARS-Cov-2) Orf1ab reverse primer of the oligo primers (ORF1ab reverse
primer)
5′- GCATCAGCTGACTGAAGCAT-3′(20-mer, nt 13433-13452, MN908947.3)
SEQ ID NO: 5 - COVID-19 (SARS-Cov-2) RNA dependent RNA polymerase forward primer of the oligo
primers (RdRP forward primer)
5′- CATGTGTGGCGGTTCACTAT- 3′(20-mer, nt 15441-15460, MN908947.3)
SEQ ID NO: 6 - COVID-19 (SARS-Cov-2) RdRP reverse primer of the oligo primers (RdRP reverse primer)
5′- TGTTAAAAACACTATTAGCATAAGCAG- 3′(27-mer, nt 15500-15526, MN908947.3)
SEQ ID NO: 7 - Human internal control-1 forward primer of the oligo primers (beta-actin forward primer)
5′- GCA CCA CAC CTT CTA CAA TGA-3′(21-mer, nt 342-362, from NM_001101.5)
SEQ ID NO: 8 - Human internal control-1 reverse primer of the oligo primers (beta-actin reverse primer)
5′- GTC ATC TTC TCG CGG TTG GC-3′(20-mer, nt 424-443 from NM_001101.5)
SEQ ID NO: 20 - COVID-19 (SARS-Cov-2) N gene (N1 region) forward primer of the oligo primers (N1
forward primer)
5′-GGGGAACTTCTCCTGCTAGAAT-3′
SEQ ID NO: 21 - COVID-19 (SARS-Cov-2) N gene (N1 region) reverse primer of the oligo primers (N1
reverse primer)
5′-TTGCTCTCAAGCTGGTTCAA-3′
SEQ ID NO: 25 - (internal control gene-2) Pbgd forward primer of the oligo primers((Pbgd forward primer)
5′- GAG ACC AGG AGT CAG ACT GT-3′(20-mer, nt 21-40, from NM_000190.4)
SEQ ID NO: 26 - (internal control gene-2) Pbgd reverse primer of the oligo primers (Pbgd reverse primer)
5′- GCT TGG AAA GTA GGC TGT GT-3′(20-mer, nt 128-147 from NM_000190.4)
[probes and potential probe sites]
SEQ ID NO: 9 - Potential site of oligonucleotide probe of N2 (77-mer, nt 28885-28951, NM_001101.5)
5′- GGAACTTCTCCTGCTAGAATGGCTGGCAATGGCGGTGATGCTGCTCTTGCTTTGCTGCTGCTTGACAGATTGAACCA -3′
SEQ ID NO: 10 - oligonucleotide probe of N2 (20-mer, nt 28929-28953, MN908947.3)
5′- TTGCTGCTGCTTGACAGATT - 3′
SEQ ID NO: 11 - Oligonucleotide probe of N2 with designated Reporter and Quencher (20-mer,
nt 28937-28956, MN908947.3)
5′-/TexRed/TTG CTG CTG CTT GAC AGA TT/BHQ_2/-3′
SEQ ID NO: 12 - Potential site of oligonucleotide probe of ORF lab (60 mer, 13373-13432, MN908947.3)
5′- TGT ACC GTC TGC GGT ATG TGG AAA GGT TAT GGC TGT AGT TGT GAT CAA CTC CGC GAA CCC - 3′
SEQ ID NO: 13 - oligonucleotide probe of ORF1ab (28-mer, nt13377-13406., MN908947.3)
5′- CCG TCT GCG GTA TGT GGA AAG GTT ATG G- 3′
SEQ ID NO: 14 - 5′-Cyanine5 and Oligonucleotide probe of Orf1ab with designated Reporter and Quencher
(28-mer, nt13377-13406.,MN908947.3)
5′-[Cyanine5]/CCG TCT GCG GTA TGT GGA AAG GTT ATG G/[BHQ2]- 3′
SEQ ID NO: 14 - 5′-FAM and Oligonucleotide probe of Orf1ab with designated Reporter and Quencher for
Triplex-1 (28-mer, nt13377-13406., MN908947.3)
5′-[FAM]/CCG TCT GCG GTA TGT GGA AAG GTT ATG G/[BHQ2]- 3′
SEQ ID NO: 15 - Potential site of oligonucleotide probe of RdRp (39-mer, nt 28883-28958,MN908947.3)
5′- ATG TTA AAA CCA GGT GGA ACC TCA GGA GAT GCC ACA AAC- 3′
SEQ ID NO: 16 - oligonucleotide probe of RdRP (25-mer, nt15471-15495, MN908947.3)
5′- CAG GTG GAA CCT CAT CAG GAG ATG C -3′
SEQ ID NO: 17 - Oligonucleotide probe of RdRP with designated Reporter and Quencher
(25-mer, nt15471-15495, MN908947.3)
5′-[FAM]/CCG TCT GCG GTA TGT GGA AAG GTT ATG G/[BHQ1]- 3′
SEQ ID NO: 18 - Human internal control-1 oligonucleotide probe (21-mer, nt 384-404, from NM_001101.5)
5′- CAC CCC GTG CTG CTG ACC GAG GC -3′
SEQ ID NO: 19 - Oligonucleotide probe of beta actin with designated Reporter and Quencher (21-mer, nt
384-404, from NM_001101.5)
5′-[HEX]/CAC CCC GTG CTG CTG ACC GAG GC/[BHQ1]- 3
SEQ ID NO: 22.- COVID-19 (SARS-Cov-2) : Potential site of oligonucleotide probe of N-1 (48 mer,
28903-28951, MN908947.3)
5′- GGCTGGCA ATGGCGGTGA TGCTGCTCTT GCTTTGCTGC TGCTTGACAG A-3′
SEQ ID NO: 23 - COVID-19 (SARS-Cov-2) N gene (N-1) oligonucleotide probe (N1 probe, 20 mer,
28934-28953, MN908947.3)
5′- TTGCTGCTGCTTGACAGATT -3′
SEQ ID NO: 24 - COVID-19 (SARS-Cov-2) N gene (N-1) oligonucleotide probe(N1 probe) with
designated Reporter and Quencher (N1 probe, 20 mer, 28934-28953, MN908947.3)
5′-TexasRed-TTGCTGCTGCTTGACAGATT-BHQ2-3′
SEQ ID NO: 27 - (internal control gene-2) Human internal control-2 oligonucleotide
probe/oligonucleotide probe of Pbgd (20-mer, nt 58-75 from NM_000190.4)
5′- CAC CCC GTG CTG CTG ACC GAG GC -3′
SEQ ID NO: 28 - (internal control gene-2) oligonucleotide probe of Pbgd
With designated Reporter and Quencher
(18-mer, nt 58-75 from NM_000190.4)
5′-/ CAC GTG TCC CCG GTA CTC/+BHQ1]- 3
[Control]
SEQ ID NO: 29 - Plasmid RNA-derived internal control -1 sequence for beta actin marker (102 mer)
5′-GGCACCACACCTTCTACAATGAGCTGCGTGTGGCTCCCGAGGAG
CACCCCGTGCTGCTGACCGAGGCCCCCCTGAACCCCAAGGCCAACCGCGAGAAGATGA-3′
SEQ ID NO: 30 - Plasmid RNA-derived internal control -2 sequence for Pbgd marker (127 mer)
5′-GAGACCAGGAGTCAGACTGTAGGACGACCTCGGGTCCCACGTGTCCCCGGTACTCGCCGGCCGG
AGCCCCCGGCTTCCCGGGGCCGGGGGACCTTAGCGGCACCCACACACAGCCTACTTTCCAAGC-3′
SEQ ID NO: 31 - Plasmid RNA-derived Positive control sequence for Orf1ab (119 mer)
Orf1ab: 5′-
CCCTGTGGGTTTTACACTTAAAAACACAGTCTGTACCGTCTGCGGTATGTGGAAAGGTTATGGCT
GTAGTTGTGATCAACTCCGCGAACCCATGCTTCAGTCAGCTGATGCACAATCGT-3′
SEQ ID NO: 45 Plasmid RNA-derived Positive control sequence for N marker (99 mer)
N: 5′-
GGGGAACTTCTCCTGCTAGAATGGCTGGCAATGGCGGTGATGCTGCTCTTGCTTTGCTGCTGCTT
GACAGATTGAACCAGCTTGAGAGCAAAATGTCTG-3′
[Target Sequences]
SEQ ID NO: 32 - RdRP target sequence

| | | | |
|---|---|---|---|
| 32 | RdRP | 15441 | catgtgtggc ggttcactat atgttaaacc aggtggaacc |
| | | 15481 | tcatcaggag atgccacaac tgcttatgct aatagtgttt ttaaca |

SEQ ID NO: 33 - Orf1ab target sequence

| | | | |
|---|---|---|---|
| 33 | orflab | 13348 | ggg ttttacactt aaaaacacag tctgtaccgt |
| | | 13381 | ctgcggtatg tggaaaggtt atggctgtag ttgtgatcaa ctccgcgaac ccatgcttca |
| | | 13441 | gtcagctgat gc |

SEQ ID NO: 34 - N-1 target sequence

| | | | |
|---|---|---|---|
| 34 | N-1 | 28881 | ggggaacttc tcctgctaga atggctggca atggcggtga |
| | | 28921 | tgctgctctt gctttgctgc tgcttgacag attgaaccag cttgagagca a |

SEQ ID NO: 35 - N-2 target sequence

| | | | |
|---|---|---|---|
| 35 | N-2 | 28861 | caactcca ggcagcagta ggggaacttc tcctgctaga atggctggca atggcggtga |
| | | 28921 | tgctgctctt gctttgctgc tgcttgacag attgaaccag cttgagagca aaatgtctgg |

SEQ ID NO: 36 - Beta actin target sequence

| | | | |
|---|---|---|---|
| 36 | beta actin | 342 | gcaccacac cttctacaat |
| | | 361 | gagctgcgtg tggctcccga ggagcacccc gtgctgctga ccgaggcccc cctgaacccc |
| | | 421 | aaggccaacc gcgagaagat ggc |

SEQ ID NO 37 - Pbgd target sequence

| | | | |
|---|---|---|---|
| 37 | Pbgd | 21 | gagaccagga gtcagactgt aggacgacct cgggtcccac |
| | | 61 | gtgtccccgg tactcgccgg ccggagcccc cggcttcccg gggccggggg accttagcgg |
| | | 121 | cacccacaca cagcctactt tccaagc |

N-3~N-6 probes (Seq ID NO: 38, 39, 40, 41, MN908947.3) wherein these primers are compatible
with all forward and reverse N primers as shown above PCR protocol change to annealing
temperature (Ta) at 58° C.;
Seq ID NO: 38 - N3 probe (20 mer)TEXAS RED-CTGGCAATGGCGGTGATGCT-BHQ2 28905-28924 bp
Seq ID NO: 39 - N4 probe (23 mer) TEXAS RED-CTGGCAATGGCGGTGATGCTGCT-BHQ2 28905-28927 bp
Seq ID NO: 40 - N5 probe (22 mer) TEXAS RED-CAATGGCGGTGATGCTGCTCTT-BHQ2 28909-28930 bp
Seq ID NO: 41 - N6 probe3 (21 mer) TEXAS RED-GATGCTGCTCTTGCTTTGCTG-BHQ2 28919-28939 bp
Beta-actin/Internal control-2 primers and probes (Seq ID NO: 42, 43, 44) wherein these primers
and probes are compatible with PCR protocol as above with no change (NM_001101.5) and show
better performance with the above primers and probes
Seq ID NO: 42 - IC-2 Forward primer (20 mer) GGCGGCACCACCATGTACCC 985-1004
Seq ID NO: 43 - IC-2 Reverse primer (20 mer) GGAGGGGCCGGACTCGTCAT 1169-1188
Seq ID NO: 44 - IC-2 probe (20 mer) HEX-CGGCGGCTCCATCCTGGCCTC-BHQ2 1107-1127

Those skilled in the art will recognize, or be able to ascertain many equivalents to the specific embodiments of the invention described herein using no more than routine experimentation. Such equivalents are intended to be encompassed by the following claims.

All publications, patents, and patent applications mentioned in this specification are incorporated herein by reference in their entireties into the specification to the same extent as if each individual publication, patent or patent application is specifically and individually indicated to be incorporated herein by reference in its entirety. Citation or discussion of a reference herein shall not be construed as an admission that such is prior art to the present invention.

1. A method for a diagnostic assay in a subject for the COVID-19 virus, natural or artificial variants, analogs, or derivatives thereof comprising:
obtaining total RNA from a biological sample, wherein the biological sample is obtained from the subject; reverse transcribing the total RNA to obtain cDNA;
subjecting the cDNA to PCR assay using a set of primers and probes; and
detecting the COVID-19 virus in the biological sample via detecting agents, thereby identifying the subject infected with the COVID-19 virus, natural or artificial variants, analogs, or derivatives thereof;
wherein the detecting agents are a COVID-19 virus having a genomic nucleic acid or nucleotides encoded by the nucleic acid sequence of SEQ ID NO:1-37;
wherein the diagnostic assay is a qualitative assay for detecting nucleic acid molecules of COVID-19 virus using reverse transcription and polymerase chain reaction (RT-PCR) or a semiquantitative testing using a titration curve, wherein the qualitative assay is a TaqMan® assay.

2. The method of claim 1, wherein the diagnostic assay further comprises primers and dual-labeled hydrolysis probes to be used in the in vitro qualitative detection of COVID-19 virus from RNA isolated from clinical respiratory specimens comprising nasopharyngeal, oropharyngeal, and nasal swabs, wherein the clinical respiratory specimens comprise upper and lower respiratory specimens.

3. The method of claim 2, wherein the RNA isolated from upper and lower respiratory specimens is purified, reverse transcribed to cDNA, subsequently amplified in a single tube in real time RT-PCR machines and associated software.

4. The method of claim 1, wherein the diagnostic assay further comprises nucleic acid molecules that are suitable for hybridization to COVID-19 nucleic acids comprising PCR primers, Reverse Transcriptase primers, probes for Southern analysis or other nucleic acid hybridization analysis for the detection of COVID-19 nucleic acids.

5. The method of claim 4, wherein the COVID-19 nucleic acids comprise the nucleic acid sequence of SEQ ID NO: 1-18 or a complement, analog, derivative, or fragment thereof, or a portion thereof; and primers comprising the nucleic acid sequence of one or more of SEQ ID 1, 2, 3, 4, 5, 6, 7, and 8.

6. The method of claim 5, wherein the nucleic acid molecules comprising the nucleic acid sequence of: (i) SEQ ID NO:1, 2, 3, 4, 7, 8, 13, and 14, or a portion thereof or (ii) SEQ ID NO: 1, 2, 7, 8, 13, 14, or a portion thereof for detecting the COVID-19 virus in the RT-PCR assay, for detecting the COVID-19 virus in a RT-PCR assay.

7. The method of claim 6, wherein the nucleic acid sequences of SEQ ID NO: 1, 2, 3, 4, 7, 8, 13, and 14 are primers, wherein the primers are Qplex.

8. The method of claim 6, wherein the nucleic acid sequence of SEQ ID NO: 1 is a primer, wherein the primer is Triplex-2.

9. The method of claim 5, wherein the nucleic acid molecule comprising the nucleic acid sequence of SEQ ID NO:1, 2, 3, 4, 15, 16, or a portion thereof is used the detection of the COVID-19 virus in a RT-PCR assay.

10. The method of claim 6, wherein the nucleic acid sequence of SEQ ID NO: 1 is a primer, wherein the primer is Triplex-1.

11. A method for diagnosing infection of COVID-19 virus in a patient, comprising:
detecting activity levels of COVID-19 virus and expression of the COVID-19 virus, natural or artificial variants, analogs, or derivatives thereof, in a sputum, nasopharyngeal aspirates, wherein the activity levels are increased activity or decreased activity of the COVID-19 virus or the expression of the COVID-19 virus in a sample relative to a control sample by contacting the upper and lower respiratory specimens comprising nasopharyngeal or oropharyngeal swabs, sputum, lower respiratory tract aspirates, bronchoalveolar lavage, and nasopharyngeal wash/aspirate or nasal aspirate with an agent which directly or indirectly detects the activity levels of the COVID-19 virus or the expression of the COVID-19 virus; and
using detecting agents, wherein the detecting agents comprise nucleic acid molecules.

12. The method of claim 12, where the detecting nucleic acid molecules are immobilized on a DNA microarray chip.

13. A diagnostic kit comprising a nucleic acid molecule for detecting a COVID-19 virus, natural or artificial variants, analogs, or derivatives thereof, wherein the nucleic acid molecule has nucleic acid sequence of SEQ ID 1,2,3,4,7,8,13,14, 9, 10, 11, 18; the nucleic acid molecule has the nucleic acid sequence of SEQ ID NO: 1,2, 7,8,13,14,9,12,18; the nucleic acid molecule has the nucleic acid sequence of SEQ ID NO: 1,2, 3,4,15,16, 9,11,17; the nucleic acid molecule has the nucleic acid sequence of SEQ ID NO:1,2,9; the nucleic acid molecule has the nucleic acid sequence of any combinations of three nucleic acid sequences: (i) SEQ ID NO. 1,2,10 or 11; (ii) SEQ ID NO: 3,4,13 or 14; (iii) Seq ID NO: 5,6, 18 or 19; (iv) COVID markers, wherein the COVID marker are SEQ ID NO: 20,21, 23 or 24; (v) Seq ID NO: 7,8,18 or 19; and (vi) a human internal control, wherein the human internal control is Seq ID NO: 25, 26, 27 or 28.

14. The diagnostic kit of claim 14 further comprises primers and a specific probe, wherein the primers comprise QPlex PCR, Triplex-1, Triplex-2, Duplex-1/2/3, and single PCR and the specific probe comprises a RdRp specific probe, a N-2 specific probe, an Orflab specific probe, an internal control beta actin specific probe, and an internal control Pbgd specific probe.

15. The diagnostic kit of claim 15, wherein the RdRP specific probe comprises a signal from a fluorescent dye on a 5′ end is quenched by BHQ-1 on a 3′ end, wherein the fluorescent dye is FAM.

16. The diagnostic kit of claim 15, wherein the N-2 specific probe comprise a signal from a fluorescent dye on a 5′ end is quenched by BHQ-2 on a 3′ end, wherein the fluorescent dye is Texas Red.

17. The diagnostic kit of claim 15, wherein the Orflab specific probe such that a signal from the fluorescent dye on the 5′end is quenched by BHQ-1 on a 3′ end, wherein the fluorescent dye is Cy-5.

18. The diagnostic kit of claim 15, wherein the internal control beta actin specific probe, the signal from the fluorescent dye on a 5′end is quenched by BHQ-1 on a 3′ end, wherein the fluorescent dye is Hex.

19. The diagnostic kit of claim 15, further comprises triplex-1 for: (i) the Orflab specific probe such that a signal from FAM on a 5′ end is quenched by BHQ-1 on a 3′ end; (ii) the N specific probe such that a signal from Texas Red on a 5′ end is quenched by BHQ-2 on a 3′ end; and (iii) the internal control Pbgd specific probe such that signal from the fluorescent dye on a 5′end is quenched by BHQ-1 on a 3′ end, wherein the fluorescent dye is Hex.

20. The diagnostic kit of claim 15 further comprises:
a multiplex real time PCR machine compatible with the diagnostic kit and a comparison genome,
wherein the multiplex real time PCR machine comprises: Rotor-Gene Q 5 plex FIRM Real Time PCR cycler, CFX96 Real Time PCR Detection System, Applied Biosystems 7500 Real Time PCR System, LineGene 9600 Plus real-time PCR detection system; and
wherein the comparison genome is GenBank MN908947.3 SARS-Cov-2 genome for determining locations of each gene markers, wherein the locations are: (i) Triplex-2, QPlex, Duplex, N-2 at 28863-28980 base pairs (bp), (ii) N-1 at 28881-28971 bp; (iii) ORFlab at 13348-13452 bp; and (iv) RdRp at 15441-15526 bp.
